(19) United States
(12) Patent Application Publication
(10) Pub. No.: US 2007/0155731 A1
(43) Pub. Date: Jul. 5, 2007

(54) Aminocyclopentyl pyridopyrazinone modulators of chemokine receptor activity

(76) Inventors: Gabor Butora, Martinsville, NJ (US); Deodialsingh Guiadeen, Linden, NJ (US); Shankaran Kothandaraman, Kendall Park, NJ (US); Malcolm MacCoss, Freehold, NJ (US); Sander G. Mills, Scotch Plains, NJ (US); Lihu Yang, Edison, NJ (US)

Correspondence Address: MERCK AND CO., INC, P O BOX 2000, RAHWAY, NJ, 07065-0907, US

(21) Appl. No.: 10/587,118

(22) PCT Filed: Jan. 26, 2005
(86) PCT No.: PCT/US05/02454

§ 371 Date: Jul. 21, 2006

Related U.S. Application Data
(60) Provisional application No. 60/539,691, filed on Jan. 28, 2004.

(51) Int. Cl. A61K 31/549; A61K 31/538; A61K 31/498; C07D 498/04; C07D 491/04; C07D 487/04

(52) U.S. Cl. 514/222.8; 514/249; 514/229.2; 544/10; 544/350; 544/65

(57) ABSTRACT

Compounds of Formula I and Formula II (wherein A, E, j, k, m, n, R¹, R², R³, R⁴, R⁵, R⁶, R⁷, R⁸, R⁹, R¹⁰, R¹⁵, R¹⁶, R¹⁷, R¹⁸, R¹⁹, R²⁴, R²⁵, R²⁶, R²⁷, R²⁸, R²⁹, R³⁰, R³¹, R³², R³³, R³⁴, X, Y and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

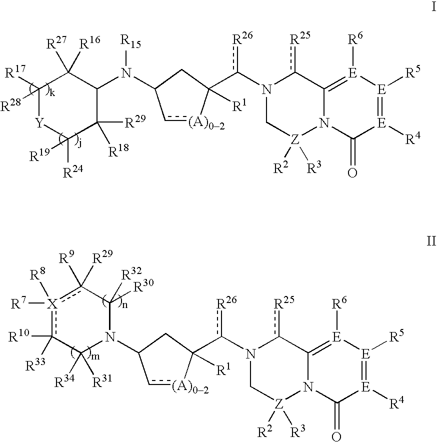

### BACKGROUND OF THE INVENTION

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANTES, MCP-1, MCP-2, MCP-3, MIP-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) [MIP-1α, MIP-1β, MCP-3, RANTES] (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC-CKR-2A”/“CC-CKR-2A”) [MCP-1, MCP-2, MCP-3, MCP-4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR-4 (or “CKR-4” or “CC-CKR-4”) [MIP-1α, RANTES, MCP-1] (Rollins, et al., Blood, 90, 908-928 (1997)); CCR-5 (or “CKR-5” or “CC-CKR-5”) [MIP-1α, RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid arthritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases.

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1−/− or CCR2 −/− mice backcrossed to APO-E −/−, LDL-R −/− or Apo B transgenic mice maintained on high fat diets (Boring et al. Nature, 394, 894-897 (1998); Gosling et al. J. Clin. Invest., 103, 773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is directed to compounds of Formula I and Formula II: 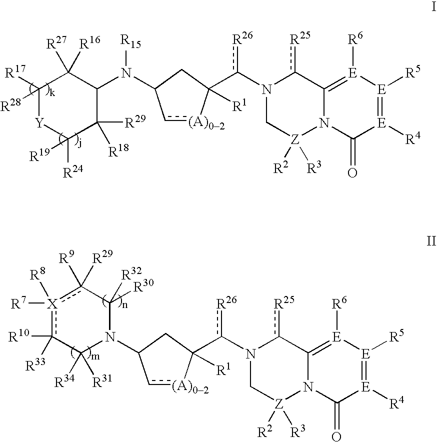
(wherein A, E, j, k, m, n, R¹, R², R³, R⁴, R⁵, R⁶, R⁷, R⁸, R⁹, R¹⁰, R¹⁵, R¹⁶, R¹⁷, R¹⁸, R¹⁹, R²⁴, R²⁵, R²⁶, R²⁷, R²⁸, R²⁹, R³⁰, R³¹, R³², R³³, R³⁴, X, Y and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is directed to compounds of Formula I and Formula II: 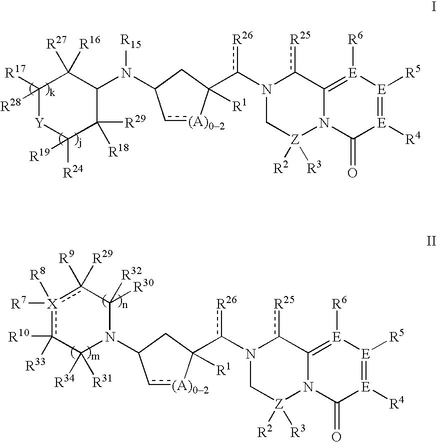
wherein:
A is selected from: —CH₂—, —O—, —N(R²⁰)—, —S—, —SO—, —SO₂—, —N(SO₂R¹⁴)—, and —N(COR¹³)—;
E is independently selected from N and C;
X is O, N, S, SO₂ or C;
Y is selected from: —O—, —N(R²⁰)—, —S—, —SO—, —SO₂—, and —C(R²¹)(R²²)—, —N(SO₂R¹⁴)—, —N(COR¹³)—, —C(R²¹)(COR¹¹)—, —C(R²¹)(OCOR¹⁴)— and —CO—;
Z is selected from C, N or O;
R¹ is selected from: hydrogen, —C₁₋₆alkyl, —O—C₁₋₆alkyl, —S—C₁₋₆alkyl, —SO—C₁₋₆alkyl, —SO₂—C₁₋₆alkyl, —SO₂NR¹²R¹², —NR¹²—SO₂—NR¹²R¹², —(C₀₋₆alkyl)-(C₃₋₇cycloalkyl)-(C₀₋₆alkyl), —CN, —NR¹²R¹², —NR¹²COR¹³, —NR¹²SO₂R¹⁴, —COR¹¹, —CONR¹²R¹², —NR¹²CONR¹²R¹², —O—CO—C₁₋₆alkyl, —O—CO₂—C₁₋₆alkyl, hydroxy, heterocycle and phenyl,
where said alkyl and cycloalkyl are unsubstituted or substituted with 1-7 substituents independently selected from: halo, hydroxy, —O—C₁₋₆alkyl unsubstituted or substituted with 1-6 fluoro, C₁₋₆alkyl unsubstituted or substituted with 1-6 fluoro, —CONR¹²R¹², —NR¹²CONR¹²R¹², —COR¹¹, —SO₂R¹⁴, —NR¹²COR¹³, —NR¹²SO₂R¹⁴, -heterocycle, ═O, —CN, phenyl, —SO₂NR¹²R¹², —NR¹²—SO₂—NR¹²R¹², —S—C₁₋₆alkyl unsubstituted or substituted with 1-6 fluoro, —SO—C₁₋₆alkyl unsubstituted or substituted with 1-6 fluoro, —SO₂—C₁₋₆alkyl, unsubstituted or substituted with 1-6 fluoro, and —O—COR¹³,
where said phenyl and heterocycle are unsubstituted or substituted with 1-3 substituents independently selected from: halo, hydroxy, —COR¹¹, C₁₋₃alkyl, and C₁₋₃alkoxy, said C₁₋₃alkyl and C₁₋₃alkoxy being unsubstituted or substituted with 1-6 fluoro;
R² and R³ are nothing when Z is O;
R² is nothing and R³ is hydrogen or C₁₋₃alkyl when Z is N;
R² and R³ are independently hydrogen or C₁₋₃alkyl unsubstituted or substituted with 1-3 fluoro, when Z is C;
R⁴ is selected from: hydrogen, C₁₋₃alkyl unsubstituted or substituted with 1-3 fluoro, —O—C₁₋₃alkyl unsubstituted or substituted with 1-3 fluoro, hydroxy, chloro, fluoro, bromo, phenyl and heterocycle, when E is C;
R⁵ is selected from: fluoro, chloro, bromo, -heterocycle, —CN, —COR¹¹, C₄₋₆cycloalkyl, —O—C₄₋₆cycloalkyl, C₁₋₆alkyl unsubstituted or substituted with 1-6 fluoro or hydroxyl or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from halo, trifluoromethyl, C₁₋₄alkyl and COR¹¹, -phenyl unsubstituted or substituted with one or more substituents selected from halo, trifluoromethyl, C₁₋₄alkyl and COR¹¹, —O-phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, and —O—C₃₋₆cycloalkyl unsubstituted or substituted with 1-6 fluoro, when E is C;
R⁶ is selected from: hydrogen, hydroxy, chloro, fluoro, bromo, phenyl, heterocycle, C₁₋₃alkyl unsubstituted or substituted with 1-3 fluoro and —O—C₁₋₃alkyl unsubstituted or substituted with 1-3 fluoro, when E is C;
R⁴ and R⁶ are independently selected from nothing or O (to make an N-oxide) when E is N;
R⁷ is selected from: hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W—C₀₋₄alkyl, (C₀₋₆alkyl)-CONR¹²-phenyl and (C₀₋₆alkyl)-CONR²³—V—COR¹¹, when X is N or C,
where W is selected from: a single bond, —O—, —S—, —SO—, —SO₂—, —CO—, —CO₂—, —CONR¹²— and —NR¹²—,
where V is selected from C₁₋₆alkyl or phenyl,
where R²³ is hydrogen or C₁₋₄alkyl, or R²³ is a 1-5 carbon linker to one of the carbons of V to form a ring,
where said C₀₋₆alkyl is unsubstituted or substituted with 1-5 substituents independently selected from: halo, hydroxy, —C₀₋₆alkyl, —O—C₁₋₃alkyl, trifluoromethyl and —C₀₋₂alkyl-phenyl,
where said phenyl, heterocycle, cycloalkyl and C₀₋₄alkyl, if present, are unsubstituted or substituted with 1-5 substituents independently selected from: halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃-heterocycle,
or where said phenyl or heterocycle is fused to another heterocycle, said other heterocycle being unsubstituted or substituted with 1-2 substituents independently selected from hydroxy, halo, —COR¹¹, and —C₁₋₃alkyl,
and where alkene is unsubstituted or substituted with 1-3 substituents which are independently selected from: halo, trifluoromethyl, C₁₋₃alkyl, phenyl and heterocycle;
   R⁷ is absent when X is O, S, or SO₂;
   R⁸ is selected from: hydrogen, hydroxy, C₁₋₆alkyl, C₁₋₆alkyl-hydroxy, —O—C₁₋₃alkyl, —COR¹¹, —CONR¹²R¹² and —CN, when X is C;
   R⁸ is nothing, when X is O, S, SO₂ or N, or when a double bond joins the carbons to which R⁷ and R¹⁰ are attached;
   or, R⁷ and R⁸ are joined to form a ring selected from: 1H-indene, 2,3-dihydro-1H-indene, 2,3-dihydro-benzofuran, 1,3-dihydro-isobenzofuran, 2,3-dihydro-benzothiofuran, 1,3-dihydro-isobenzothiofuran, 6H-cyclopenta[d]isoxazol-3-ol, cyclopentane and cyclohexane,
where said ring is unsubstituted or substituted with 1-5 substituents independently selected from:
   halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃alkyl-heterocycle;
      R⁹ and R¹⁰ are independently selected from: hydrogen, hydroxy, C₁₋₆alkyl, C₁₋₆alkyl-COR¹¹, C₁₋₆alkyl-hydroxy, —O—C₁₋₃alkyl, halo;
      or R⁹ and R¹⁰ together are O (where O is connected to the ring via a double bond);
      or, R⁷ and R⁹, or R⁸ and R¹⁰, are joined to form a fused ring which is phenyl or heterocycle, wherein said fused ring is unsubstituted or substituted with 1-7 substituents independently selected from: halo, trifluoromethyl, hydroxy, C₁₋₃alkyl, —O—C₁₋₃alkyl, —COR¹¹, —CN, —NR¹²R¹² and —CONR¹²R¹²;
      R¹¹ is independently selected from: hydroxy, hydrogen, C₁₋₆ alkyl, —O—C₁₋₆alkyl, benzyl, phenyl, C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl, and trifluoromethyl;
      R¹² is selected from: hydrogen, C₁₋₆ alkyl, benzyl, phenyl and C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl, and trifluoromethyl;
      or, when two separate R¹² groups reside on the same atom or adjacent atoms, said two R¹² groups are optionally connected via a C₁₋₇alkyl linker to form a 3 to 9 membered ring, said linker being unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
      R¹³ is selected from: hydrogen, C₁₋₆ alkyl, —O—C₁₋₆alkyl, benzyl, phenyl and C₃₋₆ cycloalkyl, where said alkyl, phenyl, benzyl, and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
      R¹⁴ is selected from: hydroxy, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
      R¹⁵ is hydrogen or C₁₋₆alkyl, where said alkyl is unsubstituted or substituted with 1-3 substituents independently selected from: halo, hydroxy, —CO₂H, —CO₂C₁₋₆alkyl, and —O—C₁₋₃alkyl;
      R¹⁶ is selected from: hydrogen, fluoro, C₃₋₆ cycloalkyl, —O—C₃₋₆cycloalkyl, hydroxy, —COR¹¹, —OCOR¹⁴, C₁₋₆alkyl unsubstituted or substituted with 1-6 substituents selected from fluoro, C₁₋₃alkoxy, hydroxyl and —COR¹¹, and —O—C₁₋₃alkyl unsubstituted or substituted with 1-3 fluoro;
      or, R¹⁵ and R¹⁶ together are a C₂₋₄alkyl or a C₀₋₂alkyl-O—C₁₋₃alkyl, forming a ring where said ring has 5-7 members;
      R¹⁷ is selected from: hydrogen, COR¹¹, hydroxy, —O—C₁₋₆alkyl unsubstituted or substituted with 1-6 substituents selected from fluoro, C₁₋₃alkoxy, hydroxy, and —COR¹¹ and C₁₋₆alkyl unsubstituted or substituted with 1-6 substituents selected from fluoro, C₁₋₃alkoxy, hydroxy, and —COR¹¹, or R¹⁷ is nothing if R²⁸ is connected to a ring carbon via a double bond;
      or, R¹⁶ and R¹⁷ together are C₁₋₄alkyl or C₀₋₃alkyl-O—C₀₋₃alkyl, forming ring where said ring has 3-7 members;
      R¹⁸ is selected from: hydrogen, fluoro, —O—C₃₋₆cycloalkyl, —O—C₁₋₃alkyl unsubstituted or substituted with 1-6 fluoro and C₁₋₆alkyl unsubstituted or substituted with 1-6 fluoro;
      or, R¹⁶ and R¹⁸ together are C₂₋₃alkyl, thereby forming a 5-6 membered ring, where said alkyl is unsubstituted or substituted with 1-3 substituents independently selected from: halo, hydroxy, —COR¹¹, C₁₋₃alkyl, and C₁₋₃alkoxy;
      or, R¹⁶ and R¹⁸ together are C₁₋₂alkyl-O—C₁₋₂alkyl, thereby forming a 6-8 membered ring, where said alkyl is unsubstituted or substituted with 1-3 substituents independently selected from: halo, hydroxy, —COR¹¹, C₁₋₃alkyl, and C₁₋₃alkoxy;
      or, R¹⁶ and R¹⁸ together are —O—C₁₋₂alkyl-O—, thereby forming a 6-7 membered ring, where said alkyl is unsubstituted or substituted with 1-3 substituents independently selected from halo, hydroxy, —COR¹¹, C₁₋₃alkyl, and C₁₋₃alkoxy;
      R¹⁹ is selected from: hydrogen, COR¹¹, SO₂R¹⁴, SO₂NR¹²R¹² and C₁₋₃alkyl unsubstituted or substituted with 1-6 substituents independently selected from fluoro and hydroxyl;
      R²⁰ is selected from: hydrogen, C₁₋₆ alkyl, benzyl, phenyl and C₃₋₆ cycloalkyl, where said alkyl, phenyl, benzyl and cycloalkyl groups are unsubstituted or substituted with 1-6 substituents independently selected from halo, hydroxy, C₁₋₃alkyl, C₁₋₃alkoxy, —CO₂H, —CO₂—C₁₋₆ alkyl, and trifluoromethyl;
      R²¹ and R²² are independently selected from: hydrogen, hydroxy, C₁₋₆ alkyl, —O—C₁₋₆alkyl, benzyl, phenyl and C₃₋₆ cycloalkyl where said alkyl, phenyl, benzyl, and cycloalkyl groups can be unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
      R²⁴ is selected from: hydrogen, COR¹¹, SO₂R¹⁴, SO₂NR¹²R¹² and C₁₋₃alkyl, where said alkyl is unsubstituted or substituted with 1-6 substituents independently selected from: fluoro and hydroxyl;
      or, R²⁴ and R¹⁷ together are a C₁₋₃alkyl bridge;
      R²⁵ and R²⁶ are independently selected from: ═O (where R²⁵ and/or R²⁶ is oxygen and is connected via a double bond), hydrogen, phenyl, and C₁₋₆alkyl substituted or unsubstituted with 1-6 substituents selected from —COR¹¹, hydroxy, fluoro, chloro and C₁₋₃alkyl;
      R²⁷ is selected from: hydrogen, COR¹¹, SO₂R¹⁴, SO₂NR¹²R¹² and C₁₋₃alkyl, where said alkyl is unsubstituted or substituted with 1-6 substituents independently selected from fluoro and hydroxyl;
      R²⁸ is selected from selected from: hydrogen, hydroxy, halo, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹² and ═O (where R²⁸ is connected to the ring via a double bond, in which case the R¹⁷ at the same position is nothing);
      R²⁹ and R³³ are selected from: hydrogen, hydroxy, C₁₋₆alkyl, C₁₋₆alkyl-COR¹¹, C₁₋₆alkyl-hydroxy, —O—C₁₋₃alkyl, trifluoromethyl and halo, or R²⁹ or R³³ are independently absent if the site of substitution is unsaturated;
      or, R²⁹ and R¹⁶ together are a C₁₋₃alkyl bridge;
      R³⁰ and R³¹ are independently selected from: hydroxy, C₁₋₆alkyl, C₁₋₆alkyl-COR¹¹, C₁₋₆alkyl-hydroxy, —O—C₁₋₃alkyl, halo and hydrogen, where said alkyl are unsubstituted or substituted with 1-6 substituents independently selected from fluoro and hydroxyl;
      or, R³⁰ and R³¹ together are a —C₁₋₄alkyl-, —C₀₋₂alkyl-O—C₁₋₃alkyl- or —C₁₋₃alkyl-O—C₀₋₂alkyl-, where said alkyl are unsubstituted or substituted with 1-2 substituents consisting of oxy (where the oxygen is joined to the bridge via a double bond), fluoro, hydroxy, methoxy, methyl or trifluoromethyl;
      R³² and R³⁴ are independently selected from: hydrogen, hydroxy, C₁₋₆alkyl, C₁₋₆alkyl-COR¹¹, C₁₋₆alkyl-hydroxy, —O—C₁₋₃alkyl, trifluoromethyl and halo;
      j is 0, 1, or 2;
      k is 0, 1, or 2;
      m is 0, 1, or 2;
      n is 1 or 2;
      the dashed line represents an optional single bond;
      and pharmaceutically acceptable salts thereof and individual diastereomers thereof.

Additional compounds of the present invention include those of Formula Ia: 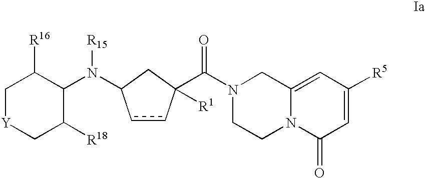
wherein R¹, R⁵, R¹⁵, R¹⁶, R¹⁸ and Y are as described herein, and pharmaceutically acceptable salts thereof and individual diastereomers thereof.

Other compounds of the present invention also include those of Formula Ib: 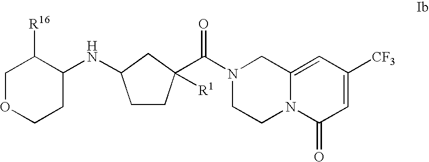
wherein R¹ and R¹⁶ are described herein, and pharmaceutically acceptable salts thereof and individual diastereomers thereof.

Certain embodiments of the present invention also include those wherein: A is CH₂; those wherein Y is O or CH₂, those wherein Y is O, those wherein E is C and/or those wherein Z is C.

Further embodiments of the present invention also include those wherein R¹ is selected from: —C₁₋₆alkyl, —C₀₋₆alkyl-O—C₁₋₆alkyl, heterocycle, and —(C₀₋₆alkyl)-(C₃₋₇cycloalkyl)-(C₀₋₆alkyl), where said alkyl, heterocycle and cycloalkyl are unsubstituted or substituted with 1-7 substituents independently selected from halo, hydroxy, —O—C₁₋₃alkyl, trifluoromethyl, C₁₋₃alkyl, —O—C₁₋₃alkyl, —COR¹¹, —CN, —NR¹²R¹², —CONR¹²R¹² and —NCOR¹³. Also included in the invention are embodiments wherein R¹ is selected from: C₁₋₆alkyl, C₁₋₆alkyl substituted with hydroxy, and C₁₋₆alkyl substituted with 1-6 fluoro. Further are embodiments wherein R¹ is selected from: —CH(CH₃)₂, —C(OH)(CH₃)₂, —CH(OH)CH₃, —CH₂CF₃.

In certain embodiments of the present invention R² is hydrogen.

In certain embodiments of the present invention R³ is hydrogen.

In certain embodiments of the present invention R⁴ is hydrogen.

In certain embodiments of the present invention R⁵ is selected from: C₁₋₆alkyl substituted with 1-6 fluoro, —O—C₁₋₆alkyl substituted with 1-6 fluoro, chloro, bromo and phenyl. Also included are embodiments of the present invention wherein R⁵ is trifluoromethyl.

In certain embodiments of the present invention R¹⁵ is methyl or hydrogen. Also included are embodiments wherein R¹⁵ is hydrogen.

In certain embodiments of the present invention R¹⁶ is selected from: hydrogen, C₁₋₃alkyl which is unsubstituted or substituted with 1-6 fluoro, —O—C₁₋₃alkyl, fluoro and hydroxy. In certain other embodiments of the present invention R¹⁶ is selected from: hydrogen, trifluoromethyl, methyl, methoxy, ethoxy, ethyl, fluoro and hydroxy.

In certain embodiments of the present invention R¹⁷ is hydrogen.

In certain embodiments of the present invention R¹⁸ is selected from: hydrogen, methyl, and methoxy. In certain other embodiments of the present invention R¹⁸ is hydrogen.

In certain embodiments of the present invention R¹⁶ and R¹⁸ together are —CH₂CH₂— or —CH₂CH₂CH₂—, thereby forming a cyclopentyl ring or a cyclohexyl ring.

In certain embodiments of the present invention R¹⁹ is hydrogen.

In certain embodiments of the present invention R²⁴ is hydrogen.

In certain embodiments of the present invention R²⁵ is hydrogen and is connected via a single bond.

In certain embodiments of the present invention R²⁶ is O and is connected via a double bond.

In certain embodiments of the present invention R²⁷ is hydrogen.

In certain embodiments of the present invention R²⁸ is hydrogen.

In certain embodiments of the present invention R²⁹ is hydrogen.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

As appreciated by those of skill in the art, halo or halogen as used herein are intended to include chloro, fluoro, bromo and iodo.

As used herein, “alkyl” is intended to mean linear, branched and cyclic carbon structures having no double or triple bonds. C₁₋₈, as in C₁₋₈alkyl, is defined to identify the group as having 1, 2, 3, 4, 5, 6, 7 or 8 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. More broadly, C_{a-b}alkyl (where a and b represent whole numbers) is defined to identify the group as having a through b carbons in a linear or branched arrangement. C₀, as in C₀alkyl is defined to identify the presence of a direct covalent bond. “Cycloalkyl” is an alkyl, part or all of which forms a ring of three or more atoms.

The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are employed. Suitable salts are found, e.g. in Remington's Pharmaceutical Sciences, 17th ed., Mack Publishing Company, Easton, Pa., 1985, p. 1418.

Exemplifying the invention is the use of the compounds disclosed in the Examples and herein.

Specific compounds within the present invention include a compound which selected from the group consisting of: the title compounds of the Examples;

and pharmaceutically acceptable salts thereof and individual diastereomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA) with and added to test compound or DMSO and ¹²⁵-I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37^{o} C. Labeled cells were washed twice and suspended at 5×10⁶ cells/ml in RPMI 1640 with L-glutamine (without phenol red) containing 0.1 mg/mil BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a certain embodiment, the disease or condition is one in which the actions of lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinophilic myositis, eosinophilic fascitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis or psoriatic arthritis.

In another aspect, the instant invention may be used to evaluate putative specific agonists or antagonists of chemokine receptors, including CCR-2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a further aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, for instance a human being, male or female, in whom modulation of chemokine receptor activity is desired. “Modulation” as used herein is intended to encompass antagonism, agonism, partial antagonism, inverse agonism and/or partial agonism. In an aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be used. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; (j) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (1) preparations of interferon beta (interferon beta-1α, interferon beta-1β); (m) other compounds such as 5-aminosalicylic acid and prodrugs thereof, antimetabolites such as azathioprine and 6-mercaptopurine, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In treating, preventing, ameliorating, controlling or reducing the risk of conditions which require chemokine receptor modulation an appropriate dosage level will generally be about 0.01 to 500 mg per kg patient body weight per day which can be administered in single or multiple doses. In certain embodiments the dosage level will be about 0.1 to about 250 mg/kg per day; or from about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, or 2.0 to 500, or 3.0 to 200, particularly 1, 5, 10, 15, 20, 25, 30, 50, 75, 100, 125, 150, 175, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, or once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made by known procedures or as illustrated.

The abovementioned modulators of chemokine activity 1-4 can be successfully synthesized by one of two principal routes. According to one of them, a protected homochiral (Eliel, E. E., Wilen, S. H., Stereochemistry of Organic Compounds, John Wiley & Sons, Inc., New York) amino acid 1-1 is, after a suitable activation, coupled with 8-trifluoromethyl-1,2,3,4-tetrahydro-6H-pyrido[1,2-a]pyrazin-6-one (1-2, Intermediate 3, or an analog thereof) and the protecting group is then removed (Greene, T., Wuts, P. G. M., Protective Groups in Organic Chemistry, John Wiley & Sons, Inc., New York, N.Y. 1991). This is illustrated in Scheme 1A.

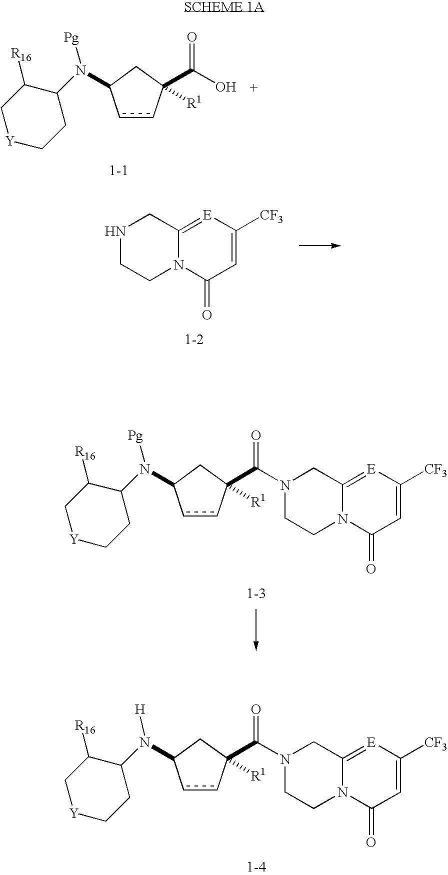

In an alternative procedure, Scheme 1B, a fully assembled homochiral 3-amino-substituted cyclopentane carboxamide 1-6 is reductively alkylated with a ketone 1-5, and if necessary, the so-formed diastereoisomers are separated by a suitable chromatography or by other physical means. The cyclopentane carboxamide can also carry a 3-oxo-group (1-8), and this could be similarly converted to the desired final compounds by a reductive amination step with a suitable, if needed, homochiral amine 1-7. This later route would invariably produce a mixture of diastereoisomers, and these could be separated using a suitable chromatography, or other physical method.

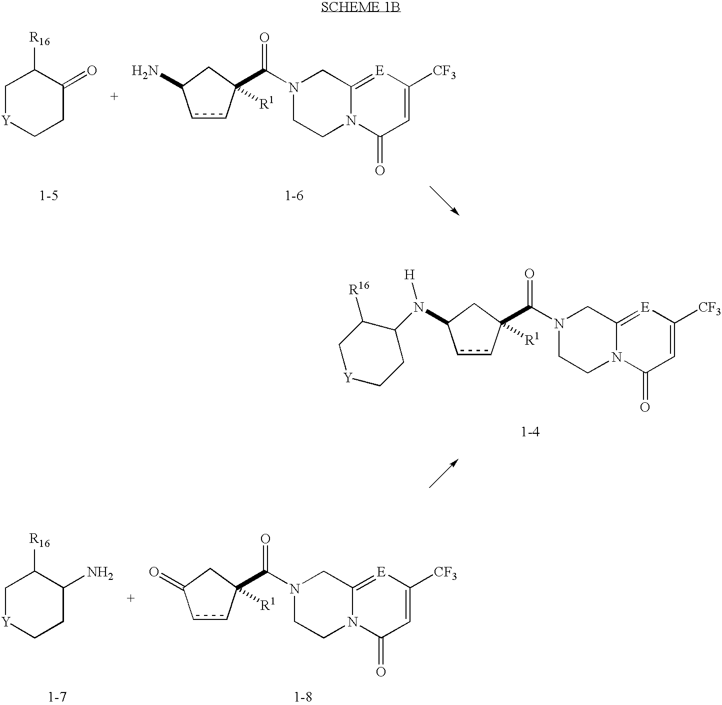

Both of these general approaches have their advantages and downsides: The advantage of the synthetic path depicted in Scheme 1A lies in the fact that the chiral synthetic steps, as well as the diastereoisomeric separations, can be performed on readily available materials at a large scale. The amide formation is performed as the penultimate step, reducing the number of synthetic operations, in which the sensitive pyridone has to be handled. On the other hand, the synthetic route depicted in Scheme 1B allows for greater variability in the synthesis and is in general shorter. However, this route requires a diastereoisomeric separation as the last step.

An example of the first general synthetic approach is depicted in Scheme 2. This synthetic sequence can be most successfully applied when the R¹ group is a simple or a branched alkyl group, for example an isopropyl, and the amine carries a simple alicyclic group with no substituent (R¹⁶=H).

According to this, the amino group of the commercially available (1R,4S)-4-aminocyclopentenecarboxylic acid is protected with a e.g. tert-butoxycarbonyl group (Greene, T., Wuts, P. G. M., Protective Groups in Organic Chemistry, John Wiley & Sons, Inc., New York, N.Y. 1991) and the double bond contained within the five membered ring is then saturated (2-3). The ester 2-4 can be produced by alkylation of the suitable acid salt with benzyl bromide, but other procedures may be suitable as well. The protecting group is removed under standard acidic conditions, and a benzophenone Schiff base is formed (2-6) to aid the subsequent introduction of the R¹ group. A base mediated C1-alkylation of 2-6 can occur either from the same side as the amino-group, giving rise to the trans-isomer, or from the opposite side (major product) producing the cis-isomer 2-7. These could be easily separated by column chromatography and the desired cis-isomer is carried forward.

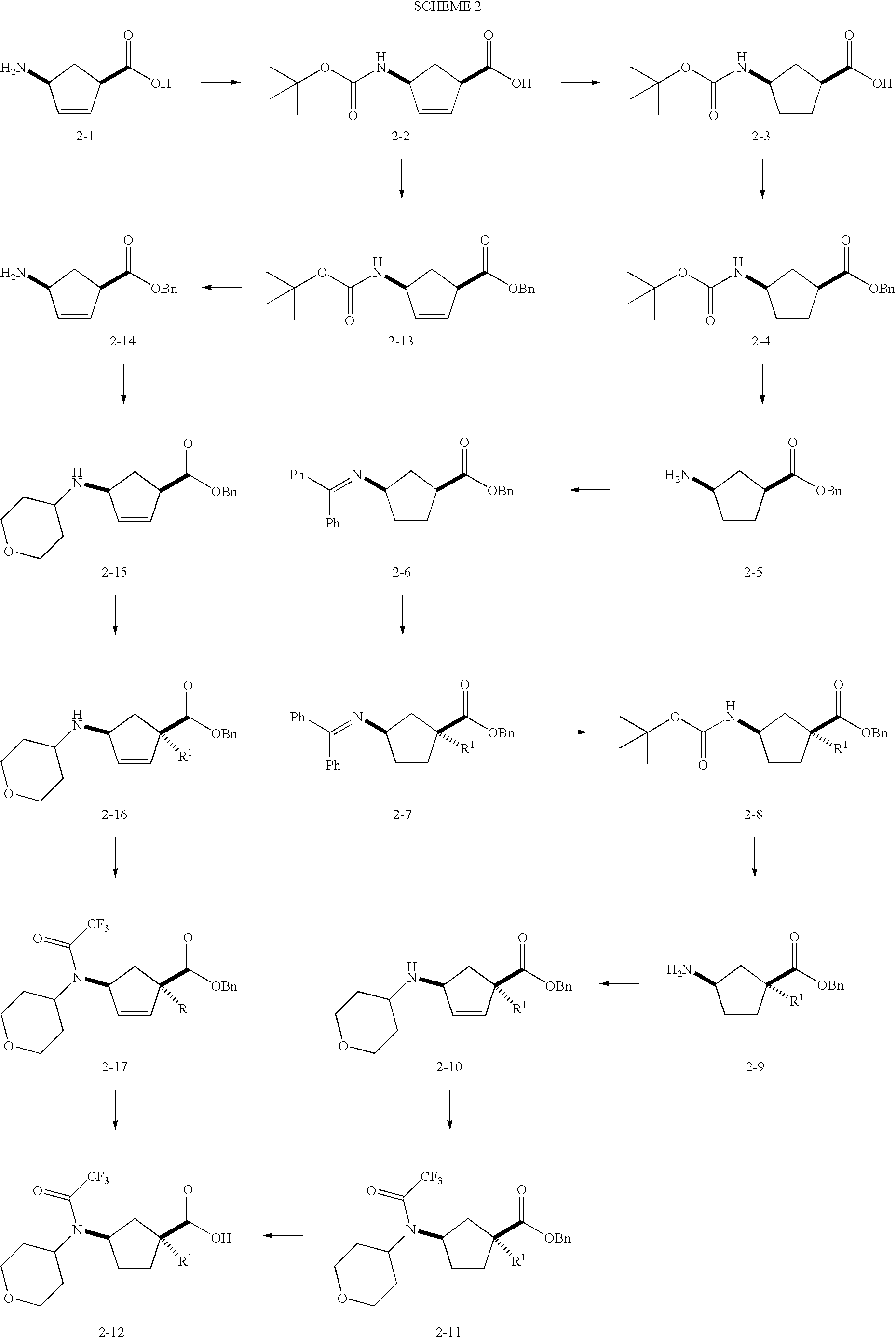

In order to facilitate isolation and purification of 2-8, an acid catalyzed cleavage of the Schiff base is followed by a standard BOC-protection (2-8) and after a suitable purification, the BOC-group is removed under the usual conditions. The amine portion of the molecule can be than completed, for example by a reductive alkylation with the appropriate ketone to afford 2-10. It is necessary to protect the secondary amine in 2-11, and a trifluoroacetyl group is particularly suitable. The benzyl ester can be cleaved by a number of procedures, and a palladium catalyzed hydrogenolysis was found to be applicable to afford 2-12.

A somewhat shorter procedure to produce 2-12 is also depicted in Scheme 2. According to this, the BOC protecting group in 2-13 is removed as described above, and a reductive amination between 2-14 and a suitable ketone, e.g. tetrahydropyran-4-one will produce the complete amine moiety. The secondary amine is then protected, e.g. as a trifluoroacetamide, and both the double bond contained within the cyclopentane core of the molecule, as well as the benzylester group are removed in a one-pot palladium catalyzed hydrogenation to yield 2-12.

In the case when R¹⁶ does represent a substituent other than hydrogen, an additional chiral center is created. This further increases the number of diastereoisomers which have to be separated during the synthetic operations. Application of the general procedure depicted in Scheme 1A is still advantageous. The order and character of the pertinent synthetic operations is similar to that described in Scheme 2 and process is illustrated in Scheme 3. According to this, the unsaturated benzyl ester 2-14 is reductively alkylated with a suitable ketone (3-methyl-tetrahydropyran-4-one in this instance) yielding 3-1. This amine represents a mixture of diastereoisomers separation of which is rather difficult at this stage. Therefore the mixture 3-1 is carried through a C1-alkylation step, in which the R¹ substituent is attached. This is accomplished by a base mediated enolate formation, followed by an alkylation with, preferably, a lower haloalkane. A number of bases can be used can be used for the generation of the enolate, potassium hexamethyldisilazane being particularly useful. As the alkylating agent can approach the enolate from either the same, or opposite sides as the amine, two sets of isomeric products (3-2) are thus formed. The separation of this mixture into its constituents presents a problem at this stage, therefore it is advantageous to carry the compound directly to the next step. The secondary amine group is protected in the form of a trifluoroacetamide by reacting 3-2 with trifluoroacetic anhydride in the presence of a suitable base. At this stage, the respective cis- and trans-isomers created in the enolate-alkylation step can be separated into two sets of diastereoisomers by means of column chromatography on silica gel. The cis-product 3-3 is then either hydrogenated to saturate the double bond as well as remove the benzyl ester protecting group to yield 3-4, or it is separated into single isomers (3-5 and 3-6) by means of preparative chiral column chromatography. The latter is achieved easily by using a Chiralpak AD column (Diacel) and a mixture of ethyl alcohol and hexane as an eluent. Just as in case of 3-3, the benzyl ester protecting group and the double bond can then be removed in a one pot hydrogenation. Alternatively, the unsaturated benzyl ester 2-14 is protected at the basic nitrogen as a tert-butyl carbamate 3-9 and this is then alkylated via its enolate as described above to afford a cis-/trans-mixture of isomers. This can be separated into single diastereoisomers by the abovementioned column chromatography on silica gel. The respective cis-isomer 3-10 is then deprotected and the resulting amine 3-11 subjected to a reductive alkylation. The secondary amine 3-12 is then protected a trifluoroacetamide 3-3, as described above.

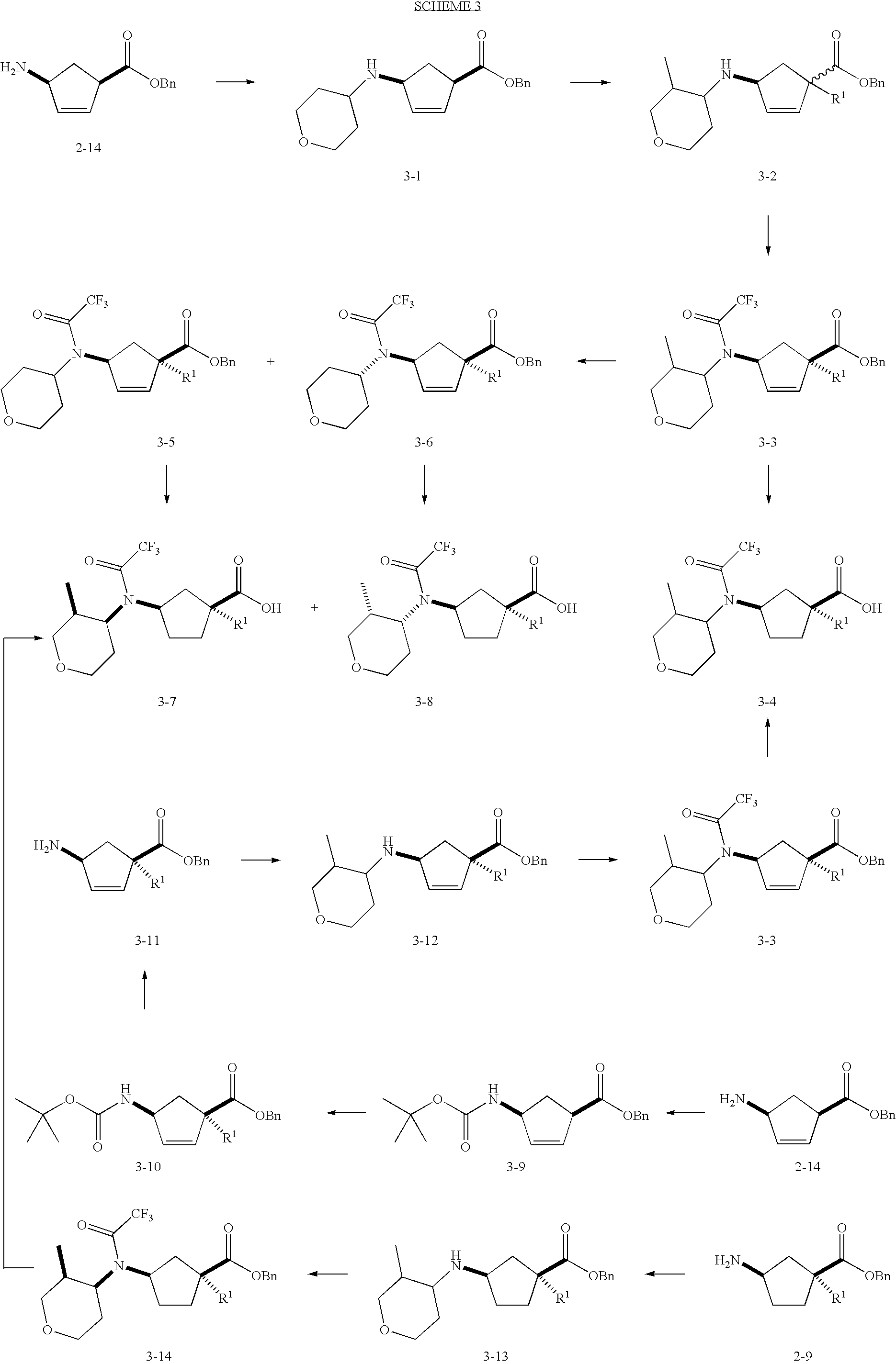

An enantiomerically pure sample of 3-7 can be also obtained by a synthetic sequence in which the benzyl ester 2-9 is reductively alkylated with the appropriate ketone, in this case 3-methyl-tetrahydropyran-4-one to afford 3-12, Scheme 3. This mixture of isomers is transformed into the respective trifluoroacetamide and these are separated by means of a carefully performed chromatographic separation on silica gel. A simple, e.g. hydrogenolytic debenzylation of 3-13 will then furnish the acid intermediate 3-7.

A great number of intermediates and examples, preparation of which is described in this document can be synthesized following the general procedure outlined in Scheme 1B. According to this, a completely assembled amino derivative 1-6 is reductively alkylated with ketone 1-5, or, alternatively, a 3-oxo-derivative 1-8 is reductively animated to yield the desired products. The former procedure is easily applicable to cases where the R¹ group is a lower alkyl, e.g. trifluoroethyl and the amide bond can be created by simple coupling procedures. The pertinent chemical steps are summarized in Scheme 4. 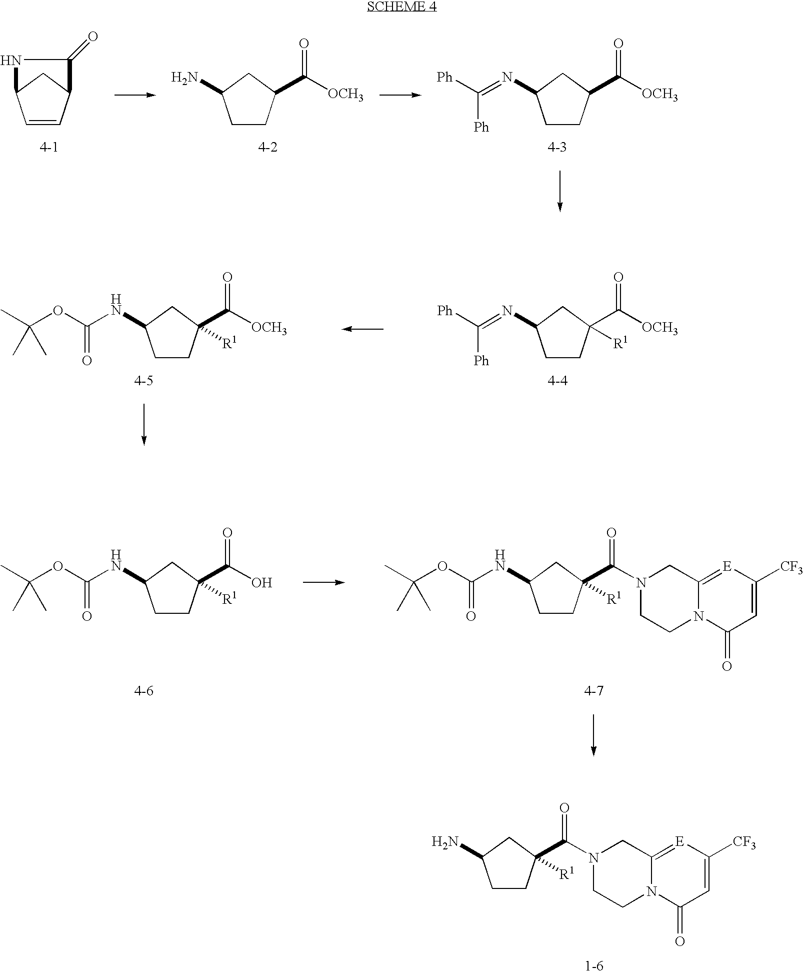
According to this, the commercially available (1S)-(+)-2-azabicyclo[2.2.1]hept-5-en-3-one (4-1) is hydrogenated to saturate the double bond present within the five membered ring, and the lactam is hydrolytically opened under acidic conditions. An acid catalyzed esterification introduces the methyl ester (4-3) and the amino group can be protected in a form of a Schiff base, as described above. The ester enolate can be formed using a strong base, e.g. lithium diisopropylamide and then alkylated with the appropriate haloalkane. The former step will scramble the stereochemistry at C1 of the cyclopentane ring, as the alkylating agent can approach the enolate from the same side-(resulting in a trans-product) or opposite side (giving rise to the cis-isomer) as the amino group at C3. The imine protecting group can be than cleaved with an acid, and the amine then re-protected with a tert-butoxycarbonyl group (4-5). At this stage the two isomers can be readily separated using a column chromatography, and the desirable cis-isomer is then carried further. A base catalyzed ester hydrolysis will liberate the carboxyl, and a standard amide bond formation will attach the isoquinolone 1-2. The BOC-protecting group can be then removed with an acid to yield 1-6.

In the case, when R¹ group in structures 1-4 represent a more complex substituted alkyl group, or, when it contains a chiral center, it is advantageous to synthesize the abovementioned modulators of chemokine activity through the completely assembled acid intermediate 1-1, Scheme 1A. An example of this procedure is described in Scheme 5. In this case, the R¹ group represents a 1-benzoyl-1-ethyl-group. According to this procedure, the 3-oxocyclopentane carboxylic acid (Stetter, H., Kuhlmann, H., Liebigs Ann. Chem., 1979, 7, 944-9) was converted to its tert-butyl ester. A number of procedures can be used for this transformation. On a small scale, the use of O-tertbutyl-N,N′-diisopropylurea is particularly advantageous. The 3-oxo group is then protected as an acetal, and the Claisen type condensation between the enolate (formed with a strong base) and acetaldehyde will furnish the desired C1-hydroxyethyl intermediate 5-3. This condensation can be successfully performed with a number of homologous aldehydes and ketones. At this stage it is advantageous to remove the acetal protecting group (acidic conditions), and subsequently protect the hydroxyl of the side chain, with, for example a benzoyl group (5-6). This intermediate contains two chiral centers, and therefore consists of two diastereoisomeric pairs (threo and erhythro). These can be successfully separated using column chromatography on silica gel and the diastereoisomeric pair containing the C1-(S)-absolute stereochemistry (5-6) is then carried further. The amine group is then completed by a reductive alkylation with a achiral or if possible a homochiral amine (1-7). The desired cis-diastereoisomers are separated using a silica gel column and this mixture of side-chain diastereoisomers is the separated into single enantiomers using a semipreparative chiralcel OD column. To ensure, that both the amine and the carboxy group can be orthogonally manipulated, the secondary amine is protected in a form of a trifluoroacetamide (5-8). The ester protecting group can be now removed, and this will furnish the penultimate acid 5-9.

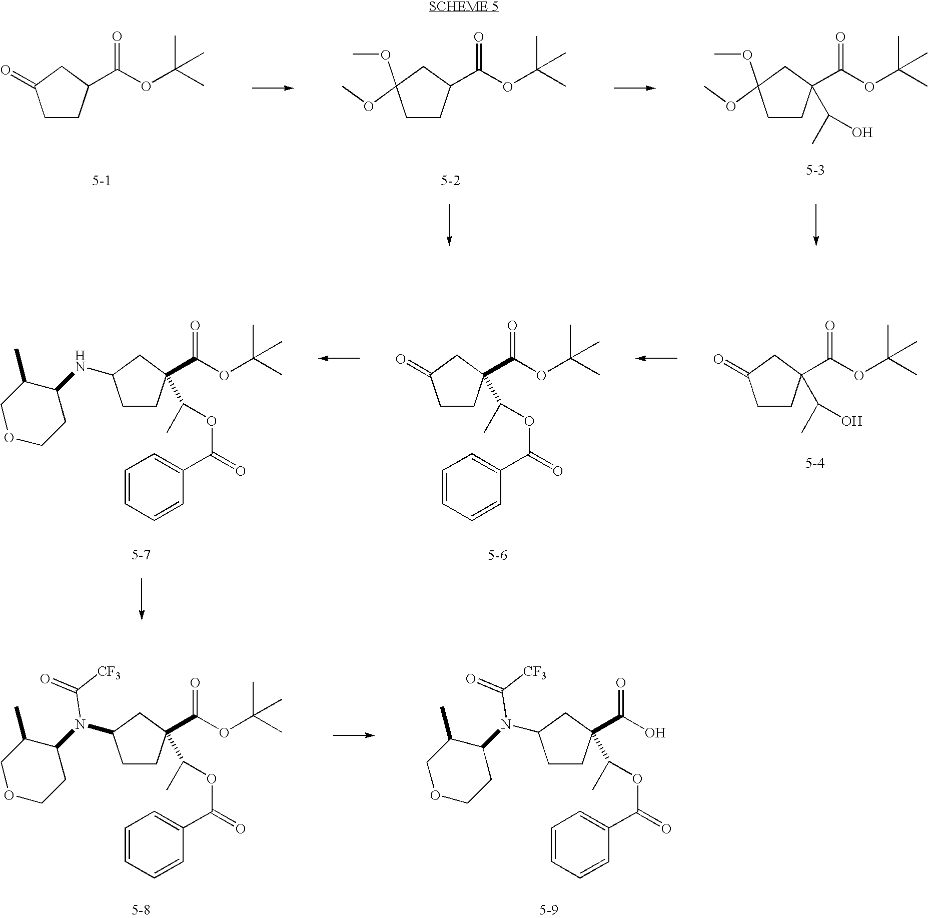

In the case, when retention of the double bond contained within the cyclopentane core is desired, it is advantageous to proceed via the path outlined in Scheme 1B. The homochiral unsaturated methyl 4(S)-aminocyclopentenecarboxylate (6-1) is according to this procedure (Scheme 6) protected in a form of a 2,5-dimethylpyrrol, which can be achieved by reacting the amine with 2,5-pentadione at elevated temperature (6-2). The enolate, which is formed with strong base is then alkylated with the suitable haloalkane, in this instance with 2-iodopropane. Once again, the desired cis-product is then separated with column chromatography and this is carried further in the synthesis. The ester can be cleaved under a number of conditions, in this case a base catalyzed hydrolysis at elevated temperatures can be successfully applied. The amide bond formation requires an activation of the acid, which can be achieved e.g. by formation of a mixed anhydride, in this case with methanesulfonyl chloride. Depending on the nature of the amine, the activated acid will react to form the desired amide at ambient or slightly elevated temperatures. The amine protecting group can be removed at this stage of the preparation with, e.g. a solution of hydroxylamine hydrochloride at elevated temperature.

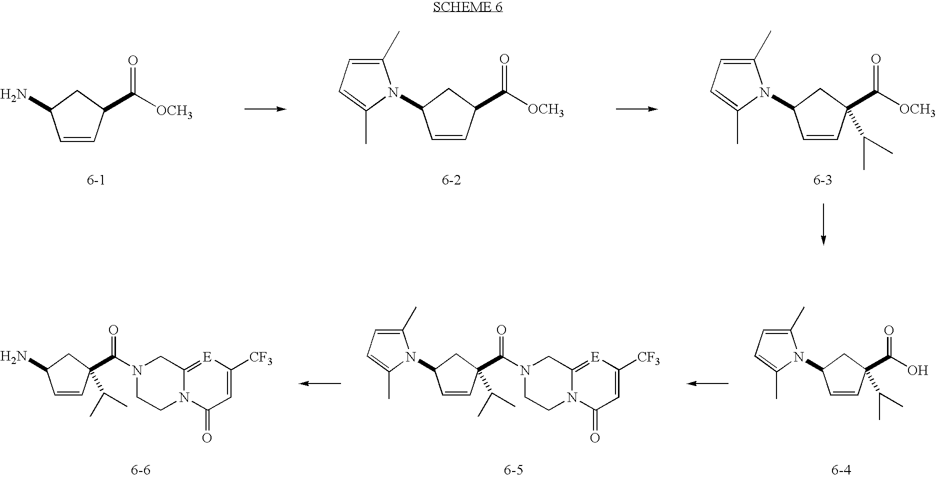

The final modulators of chemokine activity can be then synthesized by reacting these advanced intermediates with amines or ketones according to general Scheme 1A and 1B. The simple amines or ketones which are used in these transformations can be obtained either commercially or by procedures described below.

Preparation of the crucial 8-trifluoromethyl-1,2,3,4-tetrahydro-6H-pyrido[1,2-a]pyrazine-6-one is described in Scheme 7. According to one of the developed procedures (Scheme 7A) the commercially available 2,6-dichloro-4-trifluoromethyl pyridine is reacted with potassium tert-butoxide. In this transformation, one of the chlorine atoms present in the starting pyridine is displaced with the alkoxide, forming so the masked pyridine group. In the next step, the second chlorine is displaced with a cyanide group and this transformation is best performed using Pd⁰ catalysis. Hydrogenation of the nitrile then gives the aminomethyl group, and a reaction with o-nitrophenylsulfonyl chloride affords then 7-5. Given the acidic character of the sulfonamide group, a mild base (e.g. potassium carbonate) can be used to perform the desired N-alkylation with 1,2-dibromoethane, and because the masked pyridine group, the alkylating agent can be applied in large excess. Unmasking of the pyridine is performed with an acid, and a base mediated ring closure completes the second ring, 7-8. Removal of the sulfonamide is best performed with potassium thiophenolate, and to aid the product isolation, the crude material is protected with BOC₂O under standard conditions. The product can be now purified by e.g. flash column chromatography, and a standard acid catalyzed BOC-cleavage completes the synthesis.

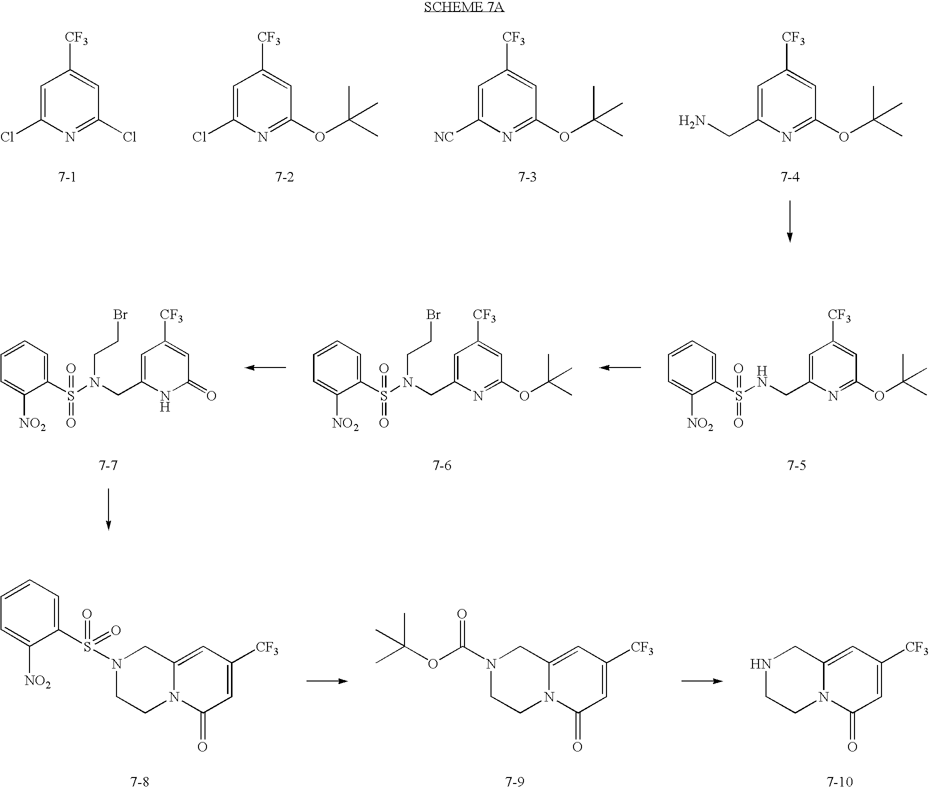

An alternative procedure is depicted in Scheme 7B. The commercially available 4-trifluoromethyl-6-chloro-2-picoline was side-chain brominated with NBS under standard conditions. The halide is then displaced with an azide group (7-13) and this is reduced to the respective amine. The sulfonamide formation and the subsequent alkylation can be then performed as described above. The subsequent displacement of the aromatic chlorine relies on activation of the neighboring nitrogen by a intramolecular ring closure. After this pyridinium species 7-17 is formed, water at elevated temperature is used to affect the desired displacement. The avoid unwanted side reactions, acidic conditions are employed during this transformation. It is also advantageous to add antioxidants, e.g. L-ascorbic acid to maintain high yields. The final set of operations is then performed as described previously.

Yet another synthetic sequence by which the pyridone 7-10 could be synthesized is described in Scheme 7° C. According to this procedure, a disubstituted acetylene in a 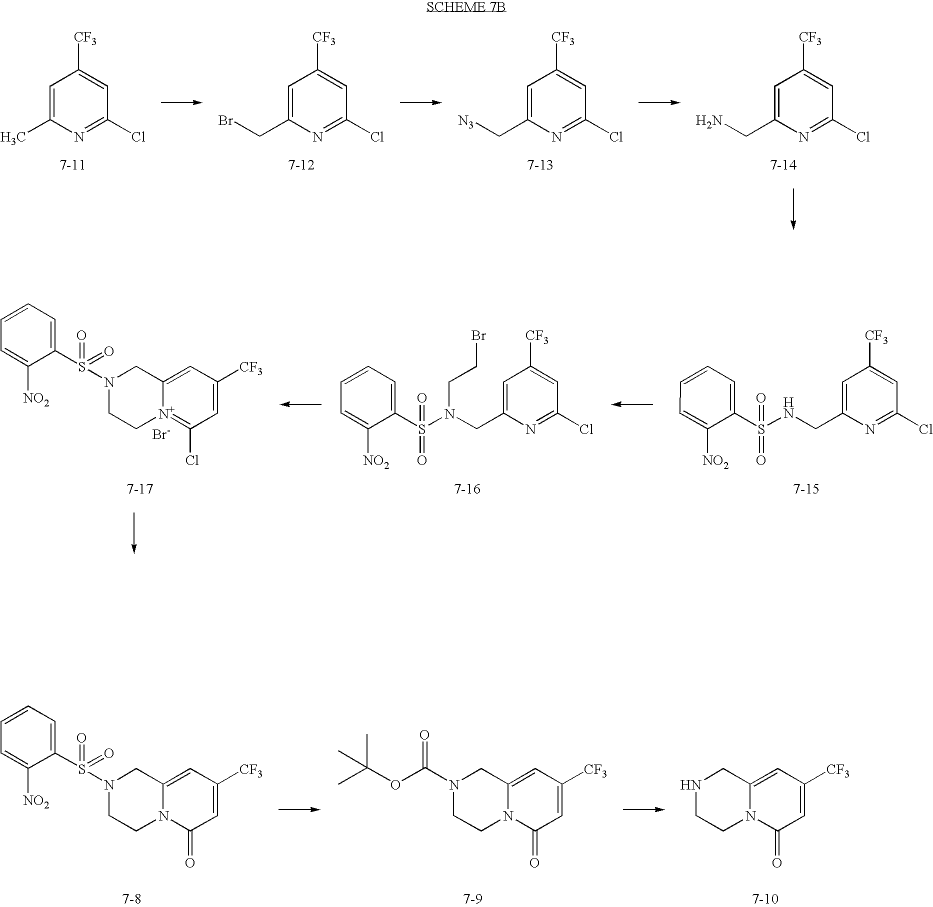
series of intramolecular nucleophilic additions cyclized to form the ring system in one tandem process. According to this, a monoprotected ethylenediamine 7-19 is reacted with nitrobenzenesulfonyl chloride 7-18 as described above. The acidic sulfonamide group is the alkylated with propargyl bromide in a presence of a weak base, e.g. potassium carbonate. A Pd⁰ catalyzed coupling of an ethyl β-iodo-γ-trifluoropropionate then introduces elements of the pyridine ring. The BOC-protecting group is now removed, and a mercury(II)chloride is then added to induce the cyclizations. The subsequent steps are identical to those described above.

Some 3-substituted analogs of 2,3,5,6-tetrahydropyran-4-one were frequently utilized during these synthetic manipulations. These compounds were either purchased, or prepared according to known procedures. However, the synthetic procedures for preparation of some, especially homochiral materials, had to be developed independently. Examples of these are illustrated in Scheme 8A and 8B.

The preparation of chiral 3-methoxytetrahydropyran-4-one is described in Scheme 8A. According to this, the commercially available 2,3,5,6-tetrahydropyran-4-one was treated with a strong base to form the respective enolate, which could be acylated to yield the respective O-benzoyl enol ether 8-2. A chiral epoxidation could be successfully 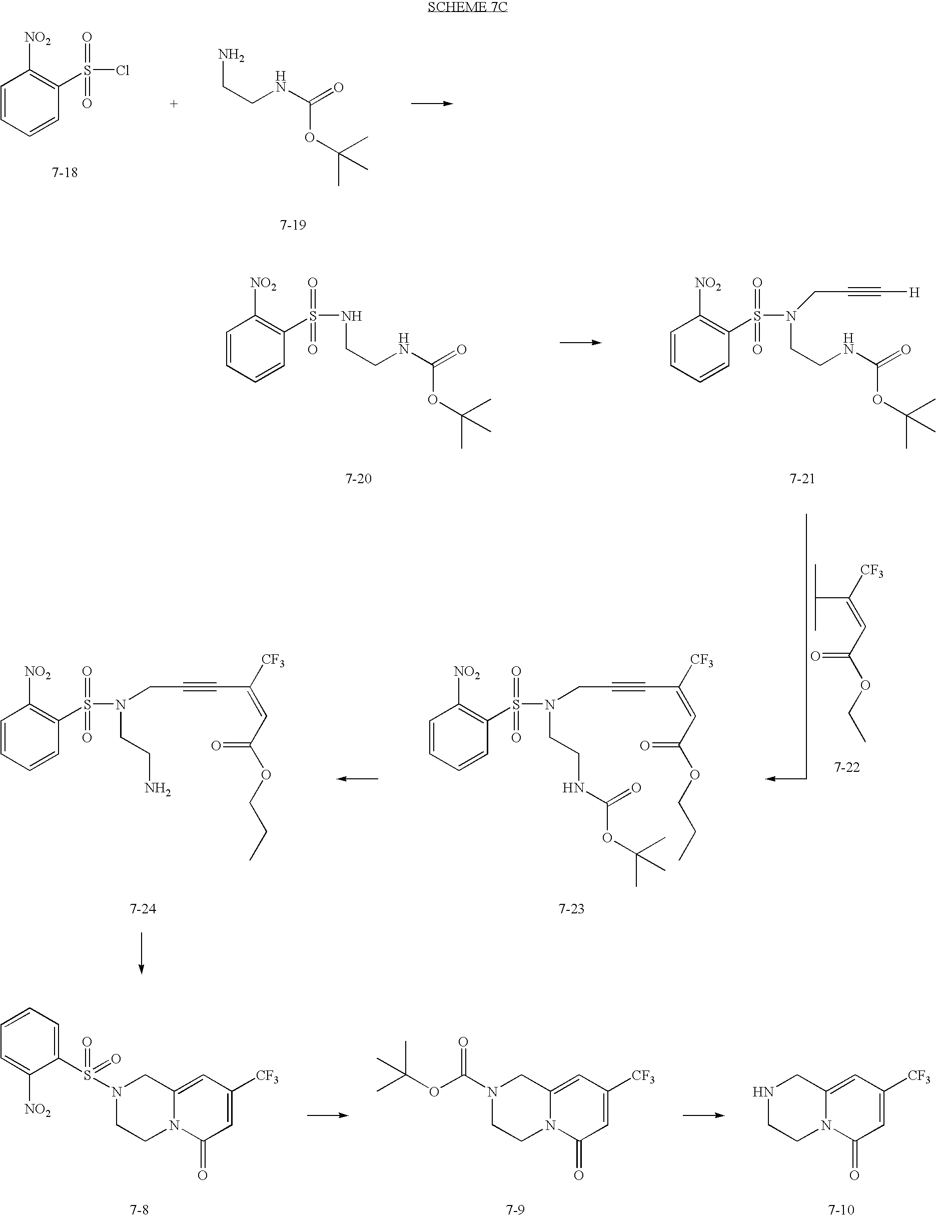
performed with L-Epoxone, and the minor, undesired enantiomer could be easily separated by chiral HPLC (Chiralpak AD). An acid catalyzed methanolysis of the homochiral epoxy-ester gave the 3-hydroxy-4,4-dimethoxy-tetrahydropyran 8-4.

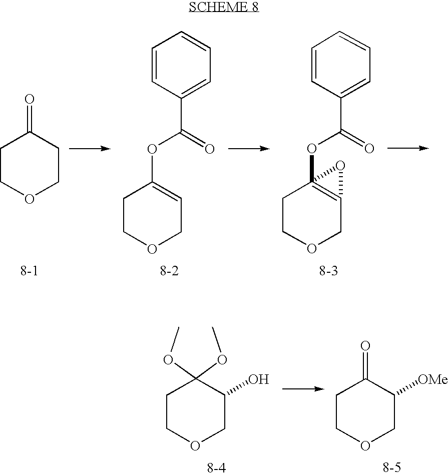

Its respective methyl ether could be then formed by a standard Williamson etherification. Clearly, this procedure is suitable for preparation of the racemate as well.

Preparation of 3-methyl-2,3,5,6-tetrahydropyran-4-one and the corresponding enantiomerically pure 4(S)-amino-3(R)-methyl-2,3,5,6-tetrahydropyran is detailed in Scheme 8B.

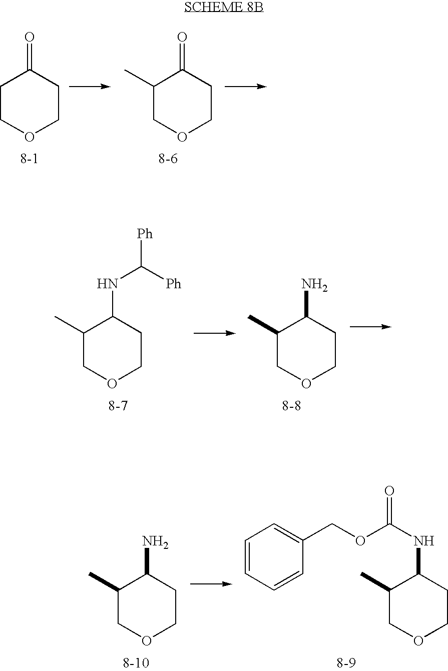

The synthetic sequence starts with the abovementioned tetrahydropyranone 8-1, which was transformed into its enolate with lithium hexamethyldisilazane and alkylated with methyl iodide. The nitrogen can be introduced by a reductive amination with benzhydrylamine, followed by catalytic scission of the benzhydryl group. The resulting amine 8-8 can be then protected, e.g. with a benzyloxycarbonyl group, and the undesired (minor) trans-isomer can be separated using column chromatography. The respective single isomers could be obtained by preparative chiral HPLC separations using Chiralpak AD columns. The cleavage of the benzoxycarbonyl group could be easily affected by hydrogen using palladium on charcoal as a catalyst.

The final modulators of chemokine activity could be than prepared following the general synthetic routes depicted in Schemes 1A and 1B using intermediates, preparation of which was described above.

The following are representative procedures for the preparation of the compounds used in the following Examples or which can be substituted for the compounds used in the following Examples which may not be commercially available.

In some cases the order of carrying out the foregoing reaction schemes may be varied to facilitate the reaction or to avoid unwanted reaction products. The following examples are provided for the purpose of further illustration only and are not intended to be limitations on the disclosed invention.

Concentration of solutions was generally carried out on a rotary evaporator under reduced pressure. Flash chromatography was carried out on silica gel (230-400 mesh). NMR spectra were obtained in CDCl₃ solution unless otherwise noted. Coupling constants (J) are in hertz (Hz). Abbreviations: diethyl ether (ether), triethylamine (TEA), N,N-diisopropylethylamine (DIEA) saturated aqueous (sat'd), room temperature (rt), hour(s) (h), minute(s) (min).

### Intermediate 1

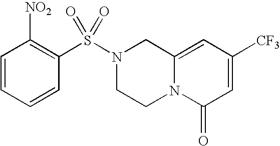 Procedure A
Step A

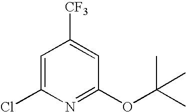

A solution of potassium tert-butoxide (13.16 g, 117.29 mmol) in anhydrous dimethyl formamide (60 mL) was cooled to 0° C. and a solution of 2,6-dichloro-4-trifluoromethyl pyridine (Lancaster, 12184) (16.89 g, 78.20 mmol) in dimethyl formamide (40 mL) was added drop-wise and the stirring was continued at 0° C. for 2 hrs. The reaction was quenched by pouring onto sat. solution of ammonium chloride (100 mL) and the crude product was extracted with hexane (3×100 mL). The combined organic phases were dried (anhydrous magnesium sulfate) and the solvent was evaporated to dryness. The product was further purified by gradient column chromatography on Silica-gel using ethyl acetate/hexane mixture as an eluent with gradually increasing concentration of ethyl acetate from 0 to 10% to yield 16.54 g (65.21 mmol, 84%). ¹H NMR (500 MHz, CDCl₃): 7.04 (s, 1H), 6.80 (s, 1H), 1.62 (s, 9H).
Step B

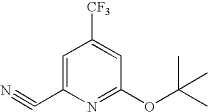

A mixture of the chloride from previous step (11.14 g, 44 mmol), zinc cyanide (10.33 g, 88 mmol) and tetrakis(triphenylphosphine)-palladium (0) (3.90 g, 3.52 mmol) in dry dimethyl formamide (50 mL) were thoroughly degassed by nitrogen/vacuum cycling and stirred at 95° C. overnight. The reaction was quenched by pouring into 200 mL of water and the product was extracted into hexane. The organic layer was filtered through a plug of Celite and evaporated to dryness to yield 12.10 g of crude product containing triphenylphosphine as the main contaminant. This residue was dissolved in tetrahydrofurane (50 mL) a solution of hydrogen peroxide in water (10 mL, 30%) was added and this mixture was stirred at room temperature for 30 minutes. The solvent was evaporated to dryness and the product was separated from triphenylphosphine oxide by column chromatography as described in Step A (ethyl acetate in hexanes, 0 to 5%). According to this procedure 4.59 g (18.79 mmol, 43%) of pure product was obtained. ¹H NMR (500 MHz, CDCl₃): 7.40 (s, 1H), 7.09 (s, 1H), 1.63 (s, 9H).
Step C

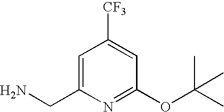

A solution of the nitrile from Step B (4.39 g, 18 mmol) and Raney Nickel (27 g) in a mixture of ethyl alcohol (160 mL) and aqueous ammonium hydroxide (40 mL) was hydrogenated in a Parr shaker at 50 psi pressure for 4 hrs. The catalyst was filtered off and the solvent was removed on a rotary evaporator. The obtained crude product (4.01 g) was used in the next step without further purification.
Step D

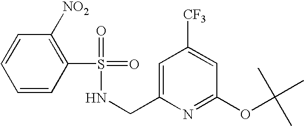

A solution of the amine from previous step (997 mg, 4.01 mmol) and diisopropyl ethyl amine (1.40 mL, 8.03 mmol) in dichloromethane (10 mL) was cooled to 0° C. and a solution 2-nitrophenylsulfonyl chloride (Aldrich) in dichloromethane (10 mL) was added via syringe. The reaction mixture was stirred without cooling for 30 minutes, and quenched with water. The crude product was extracted with dichloromethane (3×30 mL), the combined organic extracts were dried (magnesium sulfate), and the solvent was evaporated to dryness. The residue (1.9261 g) was purified on a Silica gel column as described above using a gradient of ethyl acetate from 0 to 80%. Following this procedure 964 mg (2.15 mmol, 53%) of pure material was obtained. ¹H NMR (500 MHz, CDCl₃): 8.08 (dd, J=7.3, 1.1 Hz, 1H), 7.84 (d, J=7.6 Hz, 1H), 7.70 (m, 2H), 6.92, (s, 1H), 6.73 (s, 1H), 6.25 (bt, J=5.26 Hz, 1H), 4.40 (d, J=6.0 Hz, 2H), 1.58 (s, 9H).
Step E

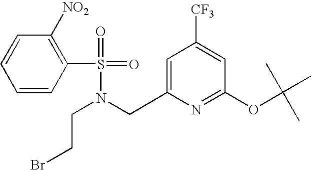

A mixture of the sulfonamide, synthesis of which was described in Step D (333 mg, 0.769 mmol), potassium carbonate (1.29 g, 15.38 mmol) in dimethyl formamide (6 mL) was treated with dibromoethane (1.44 g, 7.69 mmol) and the heated to 60° C. for 2 hrs. The reaction mixture was poured onto 30 mL of water and extracted with hexane (3×30 mL). The combined organic phases were back-washed with brine, dried with anhydrous magnesium sulfate and evaporated to dryness to yield 336.8 mg (0.623 mmol, 82%) of the pure product. ¹H NMR (500 MHz, CDCl₃): 8.02 (dd, J=7.8, 1.0 Hz, 1H), 7.70 (bm, 3H), 6.94 (s, 1H), 6.79 (s, 1H), 4.68 (s, 2H), 3.82 (t, J=7.3 Hz, 2H), 3.38 (t, J=7.6 Hz, 2H), 1.58 (s, 9H).
Step F

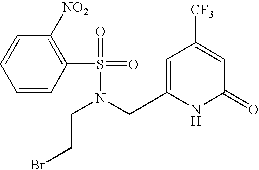

To a solution of the tert-butyl ether from previous step (330 mg, 0.611 mmol) in dichloromethane (9 mL) was added trifluoroacetic acid (1.0 mL) and the mixture was stirred at ambient temperature for 15 minutes. The solvent was evaporated to dryness, the residue diluted with hexanes, and evaporated several times to obtain 367 mg of crude product in a form of an off white solid. ¹H NMR (500 MHz, DMSO-D₆)): 8.07 (d, J=8.01 Hz, 1H), 7.97 (d, J=J=7.78 Hz, 1H), 7.86 (t, J=7.6 Hz, 1H), 7.79 (t, J=7.78 Hz, 1H), 4.55 (s, 2H), 3.81 (t, J=6.86 Hz, 2H), 3.56 (t, J=6.87 Hz, 2H).
Step G

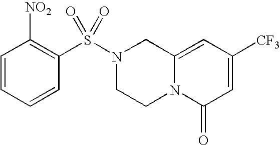

A solution of the bromide from previous step (164 mg, 0.338 mmol) in THF (12 mL) and anhydrous potassium carbonate (140 mg, 1.014 mmol) was thoroughly degassed by nitrogen/vacuum cycling stirred at ambient temperature for 3 hrs. The reaction mixture was diluted with ether (50 mL) and quenched with 10% aqueous solution of citric acid containing 3% of L-ascorbic acid. The aqueous layer was extracted 3 more times, the organic phases were combined, dried with anhydrous magnesium sulfate and the solvent was removed in vacuo to yield 108.3 mg (0.269 mmol, 80%) of pure product. ¹H NMR (500 MHz, CDCl₃): 8.09 (dd, J≈7.6, 1.6 Hz, 1H), 7.75 (m, 4H), 6.77 (s, 1H), 6.29 (s, 1H), 4.60 (s, 2H), 4.23 (t, J=5.8 Hz, 2H), 3.78 (t, J=6.0 Hz, 2H).

Procedure B

Step A

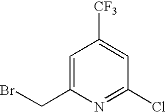

A solution of 2-chloro-6-methyl-4-trifluoromethyl pyridine (21.18 g, 108.3 mmol, Maybridge CD 10452), N-bromosuccinimide (21.20 g, 119.2 mmol, Aldrich) in tetrachloromethane (200 mL) was stirred at gentle reflux while a solution of 2,2′-azobisisobutyronitrile (1.87 mL) in tetrachloromethane (50 mL) was added, drop wise. Heating was continued for three hours, after which time the reaction mixture was allowed to cool to room temperature, washed with water (4×100 mL), dried with anhydrous magnesium sulfate and evaporated to dryness. The crude product (34 g) was purified on a Silica gel column using ethyl acetate/hexane mixture with the concentration of ethyl acetate gradually rising from 0% to 5% at the end of the separation. In this manner it was obtained: 9.6 g of 2-(α,α-dibromomethyl)-6-chloro-4-trifluoromethyl pyridine, 13.1 g of the desired 2-(α-bromomethyl)-6-chloro-4-trifluoromethyl pyridine (44%) and 9.03 g of unreacted starting material. 2-(α,α-Dibromomethyl)-6-chloro-4-trifluoromethyl pyridine: ¹H NMR (500 MHz, CDCl₃): 7.95 (s, 1H), 7.53 (s, 1H), 6.62 (s, 1H). 2-(α-Bromomethyl)-6-chloro-4-trifluoromethyl pyridine: ¹H NMR (500 MHz, CDCl₃): 7.62 (s, 1H), 7.51 (s, 1H), 4.55 (s, 2H).
Step B

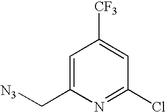

A mixture of the bromide from previous step (3.78 g, 13.62 mmol) and sodium azide (8.85 g, 136.2 mmol) in dimethyl formamide (15 mL) was stirred at room temperature under nitrogen for 24 hours. Water (50 mL) was added and the product was extracted with a mixture of hexane: diethyl ether/9:1 (3×50 mL). The combined organic phases were dried with anhydrous magnesium sulfate and the solvent was removed in vacuo. The residue (3.75 g) was further purified as described in Step 1, except that the concentration of ethyl acetate at the end of the purification reached 20%. In this manner 2.51 g (78%) of the pure product could be obtained. ¹H NMR (500 MHz, CDCl₃): 7.55 (s, 1H), 7.53 (s, 1H), 4.61 (s, 2H).
Step C

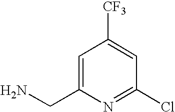

A solution of the azide from previous step (10.4 g, 44.25 mmol) and triphenylphosphine (13.93 g, 53.11 mmol) in THF (200 mL) containing 10 mL of water was stirred at room temperature overnight, after which time it was heated to 60° C. for 1 hour. The solvent was evaporated in vacuo, and the residue was dissolved in 100 mL of 2 N HCl. The non-basic side products were extracted with dichloromethane (4×50 mL), the combined organic extracts were back washed with 2N HCl. The combined aqueous phases were filtered through Celite and evaporated to dryness to leave behind the crude product in the form of a hydrochloride salt. It was used in the next step without any further purification.
Step D

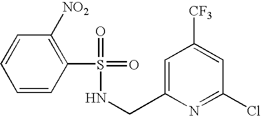

A solution of the amine hydrochloride (6.07 g, 24.57 mmol) and 2-nitrophenylsulfonyl chloride (5.44 g, 24.57 mmol) in a mixture of toluene (100 mL) and aqueous saturated sodium bicarbonate (100 mL) was vigorously stirred for 3 hrs. The organic layer was separated and the aqueous was extracted with dichloromethane. The combined organic extracts were dried with anhydrous magnesium sulfate and the solvent was removed in vacuo. The crude product (11.05 g) was further purified by flash chromatography on a silica gel column dichloromethane (100%) as eluent to yield 6.13 g (63%) of pure product. ¹H NMR (500 MHz, CDCl₃): 8.01 (dd, J=7.8, 1.4 Hz, 1H), 7.91 (dd, J=7.8, 1.1 Hz, 1H), 7.73 (dt, J=7.6, 1.4 Hz, 1H), 7.67 (dt, J=7.8, 1.4 Hz, 1H), 7.49 (s, 1H), 7.38 (s, 1H), 6.46 (t, J=6.2 Hz, 1H), 4.56 (d, J=6.5 Hz, 2H).
Step E

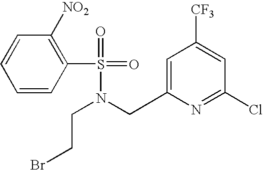

A solution of the amide from previous step (3.0 g, 7.58 mmol), dibromoethane (3.3 mL, 37.9 mmol) and dry potassium carbonate (10.47 g, 75.8 mmol) in DMF (25 mL) was stirred at 60° C. for 2 hrs. The reaction mixture was allowed to cool to room temperature and was quenched by pouring onto 10% aqueous solution of citric acid (200 mL). The product was extracted with a mixture of hexane and diethyl ether (4:1, 4×100 mL). The combined organic extracts were dried with anhydrous magnesium sulfate and the solvent was removed in vacuo. The crude product (5.91 g) was further purified by column chromatography on Silica gel, using dichloromethane (100%) as eluent. It this manner 1.876 g (49%) of the pure product could be obtained. It was used in the next reaction without delay. ¹H NMR (500 MHz, CDCl₃): 8.07 (dd, J=7.8, 1.6 Hz, 1H), 7.70 (bm, 3H), 7.49 (s, 1H), 7.44 (s, 1H), 4.82 (s, 2H), 3.82 (t, J=7.3 Hz, 2H), 3.50 (t, J=7.1 Hz, 2H).
Step F

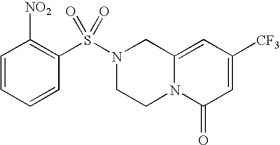

A solution of the bromide from previous step (1.87 g, 3.72 mmol) and L-ascorbic acid (1.1 g, 6.23 mmol) in a mixture of acetic acid (25 mL) and water (25 mL) was stirred at 110° C. for 1 hr. The solvent was evaporated to dryness, the residue partitioned between water (100 mL) and dichloromethane (100 mL). The aqueous phase was extracted with DCM 4 times, concentrated (2.51 g) and purified by gradient chromatography as described above using a ethyl acetate gradient 0 to 100%. The product (952 mg, 67%) was decolorized by trituration with diethyl ether to afford 899 mg of off-white solid. Both spectral as well as chromatographic behavior of this compound matched that of the standard sample.

Procedure C

Step A

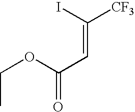

A solution of 10.0 g (60.2 mmol) ethyl 4,4,4-trifluoro-2-butynoate in diethyl ether (150 ml) at 0° C. under was treated with HI (80.0 mmol, 10.01 mL, 57% in water) and the mixture stirred at 0° C. for 1 h. After an additional 1 h at rt the reaction was quenched with aqueous sodium thiosulfate and extracted with sodium bicarbonate. The organic layer was washed with brine, dried (MgSO₄), filtered and concentrated in vacuo. The title product was obtained (without further purification) as a yellow oil, 14.6 g (82%). ¹H NMR (CDCl₃, 400 MHz): □ 7.11 (s,1H), 4.24-4.30 (q, 2H), 1.29-1.33 (t, 3H).
Step B

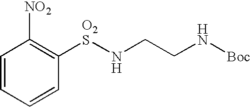

To a stirred solution of 10.0 g (62.4 mmol) t-Butyl N-(2-Aminoethyl) carbamate in anhydrous DCM (200 ml) at 0° C. under a nitrogen atmosphere was added 26.0 ml (187.2 mmol) of triethylamine followed by 2-Nitrobenzenesulfonyl chloride in small portions. The resulting clear yellow solution was then stirred for 30 min at 0° C., then at rt for 2 h. The solvent was evaporated and the resulting oil diluted with ether and extracted with water (×2). The combined organic layer was washed with brine, dried (MgSO₄) and concentrated in vacuo. The title product 21.49 g (100%) was obtained as an oil without further purification. ¹H NMR (CDCl₃, 500 MHz): □ 8.14-8.16 (m, 1H), 7.88-7.90 (m, 1H) 7.75-7.78 (m, 2H), 4.86 (b, 1H), 3.28-3.31 (t, 2H), 3.24-3.26 (t, 2H), 1.44 (s, 9H). LC-MS for C₁₃H₁₉N₃O₆S [M+H]⁺ calculated 346.10, found 346.25.
Step C

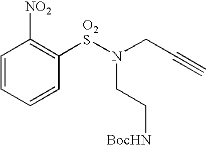

A suspension of 21.40 g (61.96 mmol) of the sulfonamide intermediate, synthesis of which was described in Step B and potassium carbonate (17.10 g, 123.9 mmol) in anhydrous DMF (200 ml) was cool to 0° C. and treated slowly with Propargyl Bromide via a syringe. The mixture was stirred at 0° C. for 30 min and at rt for 2 h. The resulting mixture was diluted with ethyl acetate and extracted with water. The combined organic layers was washed with brine, dried (MgSO₄), and concentrated in vacuo. Flash Chromatography (eluent 20% ethyl acetate/Hexane) afforded the title compound (22.76 g, 96%) as a white solid. ¹H NMR (CDCl₃, 400 MHz): □ 8.01-8.04 (m, 1H), 7.60-7.68 (m,3H), 4.47 (b,1H), 4.22 (s, 2H), 3.48-3.52 (t, 2H), 3.33-3.35 (t, 2H), 2.14 (s, 1H), 1.39 (s, 9H).
Step D

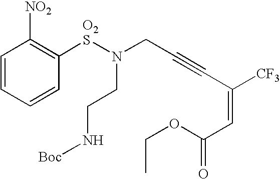

A flame dried 250 ml 3-neck round bottom flask under a nitrogen atmosphere was charged with 14.6 g (49.65 mmol) of the iodo intermediate, synthesis of which was described in Step A, 0.895 g (4.51 mmol) of copper (1) iodide, 2.62 g (2.26 mmol, 5 mole %) of [(Ph₃P)₃]₄Pd and 24.95 g (180.56 mmol) of potassium carbonate. Anhydrous THF (125 ml) was then added followed by 17.3 g (45.14 mmol) of the alkyne from Step C and the resulting mixture was stirred at 70° C. for 5 h. Excess potassium carbonate was filtered and the resulting black filtrate concentrated. Flash chromatography (eluent 15-25% ethylacetate/hexane) afforded 17.69 g (71%) of the title product as an oil. ¹H NMR (CDCl₃, 400 MHz): □ 8.00-8.02 (m, 1H), 7.64-7.59 (m,3H), 6.55 (s, 1H), 4.69 (b,1H), 4.52 (s, 2H), 4.18 (q, 2H), 3.55-3.58 (t, 2H), 3.36-3.38 (t, 2H), 1.39 (s, 9H), 1.24-1.30 (t, 3H). LC-MS for C₂₂H₂₆F₃N₃O₈S [M+H]⁺ calculated 550.14, found 450.05, (m-100).
Step E

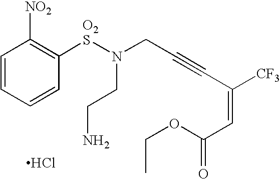

A solution of the BOC-protected amine from previous step (17.69 g, 32.19 mmol) in EtOAc (100 ml) was treated with 200 ml of a saturated solution of EtOAc/HCl at 0° C. and the mixture stirred for 2 h. The solvent was evaporated to afford 14.70 g of the title compound as tan crystalline solid (HCl salt). LC-MS for C₁₇H₁₈F₃N₃O₆S [M+H]⁺ calculated 450.09, found 450.05.
Step F

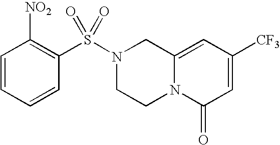

A stirred solution of alkyne from previous step (14.6 g, 30.04 mmol) in anhydrous 1,4 dioxane (100 ml) under a nitrogen atmosphere at rt was treated with 0.81 g (3.00 mmol) of mercury(II) chloride and 8.22 mL (60.08 mmol) of triethylamine. The resulting suspension was stirred at rt for 5 min then at 65° C. for 30 min and the solvent evaporated. Flash chromatography (eluent with 15-35% ethyl acetate/methyl t-butyl ethe) afforded 10.09 g (83%) of the title compound (triturated with ether). ¹H NMR (CDCl₃, 500 MHz): □ 8.09-8.12 (dd, 1H), 7.72-7.80 (m, 3H), 6.79 (s, 1H), 6.29 (s, 1H), 4.61 (s, 2H), 4.24-4.26 (t, 2H), 3.78-3.80 (t, 2H). LC-MS for C₁₅H₁₂F₃N₃O₅S [M+H]⁺ calculated 404.04, found 404.05.

### Intermediate 2

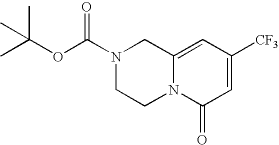

A 250 mL reaction flask was charged with potassium carbonate (8.55 g, 61.88 mmol) and flame dried under high vacuum. It was allowed to cool to room temperature under a atmosphere of nitrogen. The solid Intermediate 1 (8.32 g, 20.63 mmol) was added to it, the reaction flask was set under static atmosphere of nitrogen and DMF (50 mL) were added, via syringe. The suspension was degassed by vacuum/nitrogen cycling, and benzene thiol (2.65 mL, 25.8 mmol) were added via syringe, at room temperature. The stirring was continued at room temperature for 1 hr, after which time HPLC analysis confirmed disappearance of the starting sulfonamide. Solid BOC₂O (13.50 g, 61.88 mmol) was added, and the suspension was stirred at room temperature for additional 3 hrs. The reaction mixture was diluted with diethyl ether (300 mL), and quenched by pouring onto a solution of citric acid (100 g) and L-ascorbic acid (25 g) in 500 mL of water. The product was extracted with diethyl ether (5×100 mL), the combined extracts were dried with anhydrous sodium sulfate, and the solvent was removed in vacuo. The residue (22 g) was purified by gradient MPLC, using ethyl acetate-hexane mixture (0 to 100% of ethyl acetate) as an eluent to yield 4.95 g (75%) of pure product. ¹H NMR (CDCl₃, 500 MHz): 6.76 (s, 1H), 6.24 (s, 1H), 4.55 (s, 2H), 4.22 (t, J=5.7 Hz, 2H), 3.70 (bt, J=5.3 Hz, 2H), 1.42 (s, 9H). LC-MS for C₁₄H₁₇F₃N₂O₃[M+H⁺] calculated 319.12 found 319.10.

### Intermediate 3

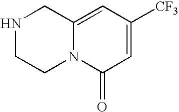

A solution of Intermediate 2 (4.54 g, 14.26 mmol) in 20 mL of 4N HCl in dioxane was stirred at room temperature for 2 hrs. The solvent was removed in vacuo, and the residue was co-distilled several times with toluene, and dried under high vacuum until no further loss of weight was noticed. This afforded the desired product (3.65 g, 100%) in a form of a HCl salt. Further purification was achieved when this solid was triturated at room temperature with 50 mL of diethyl ether and filtration (2.87 g, 79%, off white powder). LC-MS for C₉H₉F₃N₂O [M+H⁺] calculated 219.07, found 219.05.

### Intermediate 4

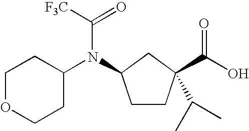 Procedure A
Step A

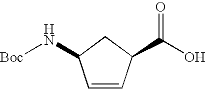

A mixture of (1R,4S)-4-amino-cyclopen-2-ene carboxylic acid (127 g, 1.0 mol), water (250 mL), sodium bicarbonate (168 g, 2.0 mol) and THF (750 mL) was stirred for 30 min, then solid Boc₂O (230 g, 1.05 mol) was added. The stirring was continued over the weekend, filtered and evaporated to remove THE. To the residue, at 0° C., was added 2N aq. HCl (˜500 mL) until pH=3.0. The resulting precipitate was collected by filtration, washed with water and dried in vacuum overnight. The desired acid was obtained in this way in a form of a white solid (227 g, 100%). ¹H NMR (400 MHz, CD₃OD): 5.95 (m, 1H), 5.79 (m, 1H), 4.80 (br s, 1H), 3.45 (m, 1H), 2.50 (m, 1H), 1.79 (m, 1H), 1.44 (s, 9H).
Step B

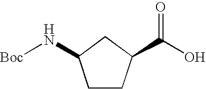

The solution of the acid (Step A, Procedure A, Intermediate 4) (227 g, 1.0 mol) and 10% Pd/C (5.0 g) in 500 mL of methanol was hydrogenated under 50 lb of hydrogen for one hour. The catalyst was removed by filtration and the filtrate was evaporated to dryness. The residue was dissolved in dichloromethane and dried over anhydrous sodium sulfate. The filtrate was evaporated to dryness and dried in vacuum. The title compound was obtained as a light yellow solid (226.0 g, 99%). LC-MS for C₁₁H₁₉NO₄ [M+H⁺] calculated 230, found 230.
Step C

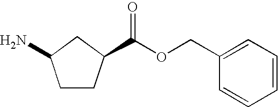

To a mechanically stirred solution of the acid (Step B, Procedure A, Intermediate 4) (226.0 g, 1.0 mol) in 500 mL of DMF was added solid potassium carbonate (210 g, 1.5 mol). The resulting mixture was stirred for 20 minutes, after which time neat benzyl bromide (118 mL, 1.0 mol) was added in one portion. An exothermic reaction was observed. After stirring for 3 h at RT, the entire mixture was poured into ice-water mixture (1000 mL) and the crude product was extracted with ether (2×800 mL). The combined organic layers were washed with water, dried over anhydrous sodium sulfate, filtered and evaporated to offer a yellow solid. This solid was mixed with 4N HCl/dioxane (400 mL), stirred overnight and concentrated. The resulting solid was collected by filtration, washed with ether and dried in vacuum. The title product was obtained as HCl salt (140 g, 55%). ¹H NMR (400 MHz, CD₃OD): 5.15 (s, 2H), 3.65 (m, 1H), 3.02 (q, J=8 Hz, 1H), 2.50 (m, 1H), 2.15 (m, 1H), 2.05 (m, 2H), 1.90 (m, 1H), 1.75 (m, 1H).
Step D

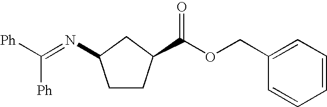

The amino benzyl ester HCl salt (Step C, Procedure A, Intermediate 4) (127 g, 0.5 mol) was suspended in 500 mL of dichloromethane. Benzophenone imine (91 g, 0.5 mol) was added. The resulting mixture was stirred overnight, filtered to remove the inorganic salt. The filtrate was washed with water and brine, dried over sodium sulfate and evaporated to dryness. The residue was dissolved in 200 mL of toluene, and evaporated again. This procedure was repeated one more time. Benzyl (1S,3R)-3-[(diphenylmethylene)amino]cyclopentanecarboxylate (178 g) was obtained as an brown oil and was used in next step without further purification. ¹H NMR (400 MHz, CDCl₃): 1.80 (m, 1H), 1.95 (m, 2H), 2.15 (m, 2H), 2.50 (m, 1H), 2.89 (m, 1H), 3.61 (m, 1H), 5.20 (s, 2H), 7.18 (d, 2H), 7.38 (m, 8H), 7.47 (m, 3H), 7.64 (d, 2H).
Step E

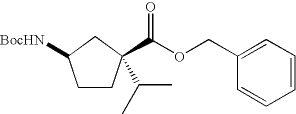

The Schiff base (Step D, Procedure A, Intermediate 2) (76.6 g, 200 mmol) in 300 mL of THF was cooled to −78° C. in a nitrogen protecting atmosphere. While stirring, a solution of LDA (2.0 M, 110 mL, 220 mmol) in heptane was added over 20 minutes. The mixture was stirred for 30 minutes at −78° C., and a solution of 68 mL of isopropyl iodide (440 mmol) in 50 mL of THF was added. The stirring was continued for another 30 minutes. The reaction temperature was allowed to raise to 0° C. by removing cooling bath. After stirring for 2 h, the entire mixture evaporated to remove THF. The residue was dissolved in ether (1000 mL), washed with water and brine, dried over sodium sulfate and evaporated. The crude product was dissolved in 500 mL of THF, mixed with 400 mL of 1N HCl, stirred for one hour, evaporated to remove THF at 50° C. The aq. solution was extracted with hexane (3×), basified with sat. aq. sodium carbonate (pH>9), mixed with a solution of Boc2O (53 g) in 500 mL of dichloromethane and stirred for 30 minutes. The organic phase was separated and the aq. phase was extracted with dichloromethane (3×). The combined organic phases were dried over sodium sulfate and evaporated. The residue was purified by flash chromatography (10% EtOAc/hexane) to yield the title compound as a mixture of cis and trans isomers (˜1:1, 24 g). Further purification on MPLC (5% EtOAc/Hexane) afforded the single cis isomer (fast-eluting, 5.0 g) and trans isomer (slow-eluting, 4.3 g). ESI-MS. for C₂₁H₃₁NO₄ calc: 361; Found: 362 (M+H)⁺.
Step F

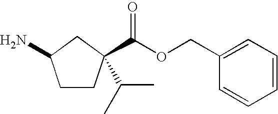

The above cis-Boc amino ester (1.25 g, 3.45 mmol) was stirred with 20 mL of 4N HCl/dioxane for one hour, evaporated and dried in high vacuum to yield benzyl (1S,3R)-3-amino-1-isopropylcyclopentanecarboxylate hydrochloride (1.05 g, 100%). ESI-MS calc. for C₁₆H₂₃NO₂: 261; Found: 262 (M+H)⁺.
Step G

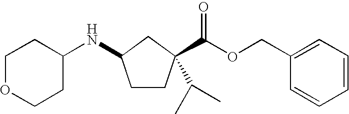

A mixture of the above amino ester (HCl salt, 1.05 g, 3.45 mmol), tetrahydro-4H-pyran-4-one (1.0 g, 10 mmol), molecular sieves (4 Å, 1.0 g), DIEA (0.78 g, 6 mmol) and sodium triacetoxyborohydride (1.33 g, 6 mmol) in 30 mL of dichloromethane was stirred overnight. The reaction was quenched with sat. aq. sodium carbonate, filtered to remove insoluble material. The crude product was extracted into dichloromethane, dried over anhydrous sodium sulfate, evaporated and dried in high vacuum. The crude product was used in next step without further purification.
Step H

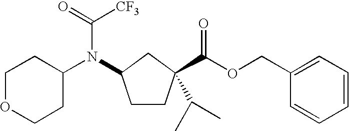

To a mixture of the crude amino ester (Step G, Procedure A, Intermediate 4) (6.85 g, 19.84 mmol), Et₃N (5.6 mL, 39.68 mmol), and DCM (50 mL), was slowly added TFAA (6.91 mL, 49.6 mmol). The reaction was stirred at room temperature for 1 hour. It was washed with 1N HCl and brine, dried over anhydrous MgSO₄, and concentrated in vacuo. The crude product was purified by MPLC (20/80, EtOAc/Hexanes) to yield the title compound (3.7 g, 42.2%). LC-MS for C₂₃H₃₁F₃NO₄ [M+H⁺] calculated 442.21, found 442.3.
Step I

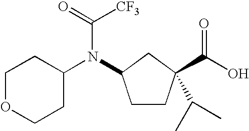

A mixture of the amide (Step H, Procedure A, Intermediate 2) (4.7 g, 10.7 mmol), 10% Pd/C (500 mg), and MeOH (50 mL) was stirred under a hydrogen balloon for 2 hours before filtered through celite and concentrated in vacuo to yield the target acid (3.92 g, 99%). LC-MS for C₁₆H₂₅F₃NO₄ [M+H⁺] calculated 352.17, found 352.15.

Procedure B

Step A

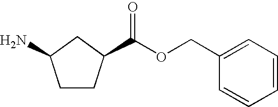

To a magnetically stirred solution of the Boc-amino acid (Step A, Procedure A, Intermediate 4) (159 g, 0.7 mol) in 500 mL of DMF was added solid potassium carbonate (138 g, 1.0 mol). The resulting mixture was stirred for 20 minutes, a neat benzyl bromide (84 mL, 0.7 mol) was added in one portion. An exothermic reaction was observed. After stirred overnight at RT, the entire mixture poured onto ice-water mixture (1000 mL). The crude product was extracted with ethyl acetate (2×800 mL). The combined organic layers were washed with water, dried over sodium sulfate, filtered and evaporated to offer a brown oil. This material was mixed with 4N HCl/dioxane (350 mL) and stirred until no gas evolution was observed. Ether (500 mL) was added, the precipitate was collected by filtration and washed with ether and hexane. The desired product was obtained as HCl salt (164 g, 93% ). ¹H NMR (400 MHz, CD₃OD): 7.38 (m, 5H), 6.25 (m, 1H), 5.94 (m, 1H), 5.20 (s, 2H), 4.32 (br s, 1H), 3.80 (br s, 1H), 2.67 (m, 1H), 2.14 (m, 1H).
Step B

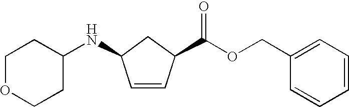

To a mixture of the amino ester HCl salt (Step A, Procedure B, Intermediate 4) (38 g, 150 mmol), tetrahydro-4-H-pyran-4-one (15 g, 150 mmol), DIEA (20.6 g, 160 mmol) and molecular sieves (4 Å, 20 g) in 200 mL of dichloromethane was added sodium triacetoxy borohydride (42.4 g, 200 mmol) in multiple portions. After complete addition, the mixture was stirred at RT overnight, quenched with sat. aq. sodium carbonate, filtered through celite. The crude product was extracted into dichloromethane (3×), dried over sodium sulfate and evaporated. The residue was purified by flash chromatography (aq. NH4OH+MeOH/1:9)/DCM (1:9)). The desired fractions were combined and evaporated. The residue was mixed with THF and evaporated, dissolved in toluene and evaporated and dried in vacuum to yield a light brown oil (38 g, 84%). ¹H NMR (400 MHz, CDCl₃): 7.38 (m, 5H), 5.98 (m, 1H), 5.85 (m, 1H), 3.98 (m, 3H), 3.54 (m, 1H), 3.40 (m, 2H), 2.82 (m, 1H), 2.44 (m, 1H), 1.90 (m, 1H), 1.79 (m, 2H), 1.70 (m, 1H), 1.44 (m, 2H).
Step C

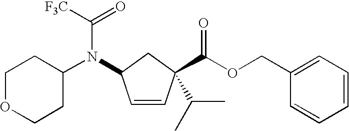

To a round flask containing solid potassium bis-(trimethylsilyl) amide (30 g, 151 mmol) under nitrogen was added 500 mL of anhydrous THF, cooled at −78° C. A solution of the amino ester (Step B, Procedure B, Intermediate 4) (38 g, 126 mmol) in 100 mL of THF was added in 20 minutes. The dry ice-acetone bath was changed into a dry ice-water (˜−15° C.). The mixture was stirred at −15° C. for one hour and cooled to −78° C. again. A neat solution of isopropyl iodide (65 mL, 378 mmol) was added. The flask was placed into −15° C. bath. After a few minutes, a formation of large amount of white precipitate was observed. The reaction mixture was stirred for additional one hour, poured into a mixture of ice and water, extracted with ether (3×). The ether layers were washed with water and brine, dried over sodium sulfate and evaporated. The residue was dissolved in dichloromethane, dried over sodium sulfate again and evaporated. The solution of the crude product in dichloromethane (200 mL) was cooled to 0° C. To this solution was added pyridine (33 mL, 400 mmol) and trifluoroacetic anhydride (27 mL, 190 mmol), drop wise. After one hour, the reaction was quenched with water. The organic phase was separated and washed with 2N aq. HCl, water and brine. The crude product was purified by flash chromatography (20% EtOAc/hexane) to yield a light brown oil (41 g, 74%). ¹H-NMR indicated a 5:1 mixture of cis/trans isomers. ¹H NMR (400 MHz, CDCl₃): Cis-Isomer: 6.06 (m, 1H), 5.68 (m, 1H), trans: 5.92 (m, 0.2H), 5.79 (m, 0.2H). LC-MS for C₂₃H₂₈F₃NO₄ [M+H⁺] calculated 440, found 440.
Step D

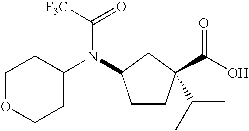

The unsaturated benzyl ester (Step C, Procedure B, Intermediate 4) (41 g) and 10% Pd/C (2.0 g) in ethyl acetate (100 mL) was hydrogenated under 50 psi of hydrogen overnight. The catalyst was removed by filtration through a pad of celite. The filtrate was evaporated and dissolved in dichloromethane, evaporated and dried in vacuum overnight. The desired acid was obtained as a gummy white solid (32.5 g, 100%). LC-MS for C₁₆H₂₄F₃NO₄ [M+H⁺] calculated 352, found 352.

### Intermediate 5

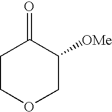 Procedure A
Step A

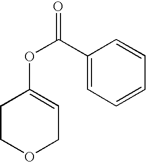

To a solution of Bz₂O (58.8 g, 259.8 mmol), DMAP (1.3 g, 10.8 mmol), THP ketone (20 mL, 216.5 mmol) in THF (600 mL) was added KHMDS (0.5 M solution in toluene, 520 mL) at 16° C. (a water bath) over 60 minutes via canula. After addition, the suspension was further stirred for one hour before concentrated to about 200 mL in vacuo. The reaction was then quenched with saturated NaHCO₃ aqueous solution (500 mL). The mixture was partitioned between hexane and water. The aqueous layer was separated and further extracted with hexane (3×400 mL). The organic layers were combined, dried over anhydrous Na₂SO₄ and concentrated. The desired product 1 (21 g, 47%) was distilled out from the remaining oil (115-118° C., 1.0 mmHg). H1 NMR (500 MHz, CDCl₃) δ 8.09 (d, J=15 Hz, 1H)), 7.75-7.40 (m, 3H), 5.61 (bs, 1H), 4.30 (bs, 2H), 3.99 (t, J=11 Hz, 2H), 2.45 (bs, 2H).
Step B

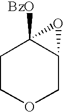

A mixture of the enol ether from Step A (12.34 g, 60.5 mmol), Bu₄HSO₄ (820 mg, 2.42 mmol), CH₃CN (750 mL), a buffer solution (500 mL of 0.05M Na₂B₄O₇ in 4×10⁻⁴ M Na₂EDTA), and D-Epoxone® (4.7 g, 18.1 mmol) was stirred in an ice-bath using mechanical stirrer. To this ice-cold mixture was simultaneously added a solution of Oxone (52 g, 84.7 mmol) in 250 mL of 4×10⁻⁴ M Na₂EDTA solution and a solution of K₂CO₃ (48.5 g, 350 mmol) in water (250 mL) over 1.5 hr in an ice bath using two addition funnels. After addition, the mixture was stirred for another 0.5 hr before partitioned between ether (1.5 L) and H₂O (1 L). The aqueous layer was separated and further extracted with ether (3×1 L). The organic layers were combined, dried over anhydrous Na₂SO₄, concentrated and purified by flash chromatography (15% EtOAc/hexane) to give the desired epoxide 2 (6˜7 g, 50˜60%, 80% ee). Some starting material may be eluted out together with the product. This impurity can be removed after chiral HPLC separation (AD column). Rf=0.2 (50% EtOAc/hexane). NMR (500 MHz, CDCl₃) H1 δ 8.05 (d, J=8 Hz, 1H), 7.65-7.20 (m, 3H), 4.05 (dd, J=13.7 Hz, 2.1 Hz 1H), 3.94 (d, J=13.7 Hz, 1H), 3.62 (m, 2H), 3.45 (d, J=1.5 Hz), 2.52 (m, 1H), 2.28 (m, 1H); C13 δ 200.4, 165.0, 133.7, 129.9, 128.6, 80.9, 64.8, 62.4, 56.6, 29.0.
Step C

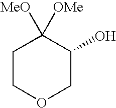

To a solution of epoxide from the previous step (11 g, 100% ee, 50 mmol) in CH₂Cl₂ (120 mL) and anhydrous methanol (38 mL) was added CSA (580 mg, 2.5 mmol) at room temperature. After 4 hours, the reaction was quenched with triethyl amine (1.05 mL) and then concentrated to oil. This oil was directly purified on MPLC (50-60% EtOAc/hexane) to give 3 (8 g, 99%). H1 NMR (500 MHz, CDCl₃) δ 3.85-3.80 (m, 2H), 3.74-3.68 (m, 2H), 3.50 (dt, J=14, 1.8 Hz, 1H), 3.29 (s, 3H), 3.28 (s, 3H), 2.0-1.95 (m, 1H), 1.85-1.78 (m, 1H).
Step D

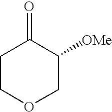

To a suspension of NaH (2.37 g of 95%, 98.8 mmol) in THF (200 mL) was added a solution of the alcohol from step C (8 g, 49.4 mmol) in THF (30 mL) very slowly in an ice-bath. At the gas evolution was finished, the ice-bath was removed and MeI (9.2 mL, 98.8 mmol) was added. The reaction was stirred at room temperature overnight. TLC indicated completed reaction. The reaction was then quenched with concentrated aq. HCl (2 mL of 37%, ˜20 mmol) until PH˜7. Water (5 mL) was then added. Another 2 mL of concentrated aq. HCl was added to make PH˜1. The reaction was stirred for another 1 hr and TLC indicated completed hydrolysis of the dimethyl ether. The solution was directly loaded to a large silica gel column and eluted with ethyl acetate to give the desired methoxy ketone 5 (5.5-6 g, 85%-90%).

### Intermediate 6

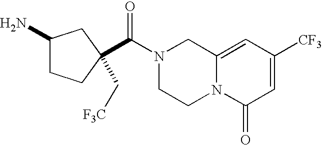 Step A

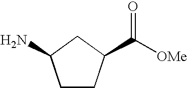

A mixture of (1S)-(+)-2-azabicyclo[2.2.1]hept-5-en-3-one (10.3 g, 94.4 mmol) in ethyl acetate (200 mL) and 10% Pd/C (0.5 g), was hydrogenated at room temperature. After 24 h the reaction mixture was filtered and evaporated leaving behind 10.4 g (100%) of the crude product. This was taken in 250 mL methanol and HCl (12 M, 6 mL) was added. The resultant mixture was stirred at room temperature, until the reaction was complete (72 h). The solvent was evaporated and the crude product was dried under high vacuum to yield the title compound as an off white solid (16.0 g, 96%). ¹H NMR (500 MHz, D₂O): δ 3.70 (s, 3H), 3.01 (m, 1H), 2.38 (m, 1H), 2.16-1.73 (m, 6H).
Step B

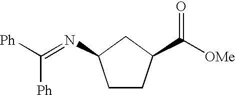

To a suspension of the ester intermediate from Step A (10.2 g, 56.8 mmol) in dry dichloromethane (200 mL) was added benzophenone imine (10.2 g, 56.8 mmol) and the resultant mixture was stirred for 24 h at room temperature. The reaction mixture was filtered and the filtrate was evaporated. The remaining oil was triturated with ether (100 mL), filtered and evaporated. The precipitated ammonium chloride was filtered and this operation was repeated two more times to ensure that the product was free of ammonium chloride. The resultant oil was thoroughly dried under vacuum to yield the title compound (18.03 g, >100%) and required no further purification. ¹H NMR (500 MHz, CDCl₃): δ 7.5-7.18 (m, 10H), 3.75 (m, 1H), 3.7 (s, 3H), 2.78 (m, 1H), 2.26-1.71 (m, 6H).
Step C

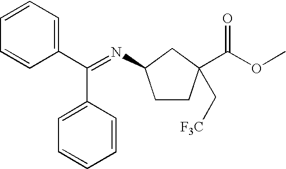

A flame dried 1000 mL round bottom flask was charged with 400 mL of dry tetrahydrofuran, set under nitrogen and cooled to −78° C. using an acetone/dry ice bath. Diisopropylamine (27.4 mL, 195 mmol) was added via syringe. The resulting solution was slowly treated with n-butyllithium (55 mL, 140 mmol, 2.5 M in hexanes). After 5 min stirring, the imine, preparation of which was described in Step B (40 g, 130 mmol) in 100 mL of tetrahydrofuran was added drop-wise via syringe and the resulting mixture was stirred at −78° C. for 2 h. 2-Iodo-1,1,1-trifluoroethane (47 mL, 480 mmol) was then added drop-wise via syringe and the resulting mixture was stirred overnight allowing it to warm slowly to room temperature. The reaction was quenched with a saturated solution of ammonium chloride (400 mL) and the organics were separated. The aqueous layer was extracted with ethyl acetate (3×150 mL), the organic extracts were combined, dried over anhydrous sodium sulfate, filtered and evaporated under reduced pressure. The crude product was used in the next step without further purification. LC-MS for C₂₂H₂₂F₃NO₂ calculated 389.26, found [M+H⁺] 390.4
Step D

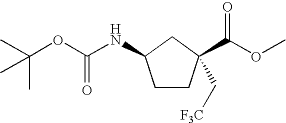

To a solution of the product from Step C (130 mmol, assuming 100% conversion) in 200 mL of tetrahydrofuran was added 200 mL of 2 N hydrochloric acid and the resulting mixture was stirred overnight at room temperature. The solution was concentrate in vacuo to remove most of the tetrahydrofuran and diluted with dichloromethane (300 mL). The pH of the aqueous layer was adjusted to 10 by the slow addition of 5 N sodium hydroxide with vigorous stirring. The organic layer was separated and the aqueous layer was extracted with dichloromethane (2×150 mL). The organic extracts were combined, dried over anhydrous sodium sulfate, and filtered. To the filtrate was added diisopropylethylamine (22.7 mL, 130 mmol) and di-tert-butyl dicarbonate (32.7 g, 150 mmol) and the resulting solution was stirred at room temperature overnight. The mixture was washed with 1 N hydrochloric acid, followed by a saturated solution of sodium bicarbonate, and brine. The organic layer was dried over anhydrous sodium sulfate, filtered, and evaporated to dryness under reduced pressure. Purification by MPLC (in several batches, about 5 g per run) afforded 5.87 g (14%) of the desired cis (R, S) isomer and 12.31 g (29%) of the trans (S, S) isomer along with 5.22 g (12%) of a 1:1 mixture of the 2 diastereomers. ¹H NMR (500 MHz, CDCl₃) δ (1^{st} desired isomer) 5.05 and 4.40 (singlets, 1H), 3.76 (s, 3H), 2.73 (ddd, J=11.0, 12.8, 14.8 Hz, 1H), 2.38 (ddd, J=10.7, 12.8, 15.0 Hz, 1H) 2.32-2.26 (m, 1H), 2.21 (br dd, J=3.6, 14.5 Hz, 1H), 2.18-2.11 (m, 1H), 2.02 (dd, J=8.8, 14.4 Hz, 1H), 1.61 (dd, J=7.8, 13.2 Hz, 1H) 1.52 (br s, 10H). ¹H NMR (500 MHz, CDCl₃) δ (2^{nd} isomer) 4.52 and 4.06 (singlets, 1H), 3.72 (s, 3H), 2.72 (dd, J=7.1, 13.5 Hz, 1H), 2.66 (ddd, J=10.6, 12.8, 15.0 Hz, 1H), 2.53 (ddd, J=11.0, 12.8, 14.9 Hz, 1H) 2.26 (app dd, J=7.1, 13.5 Hz, 1H), 2.18-2.07 (m, 1H), 1.78 (dd, J=8.6, 13.5 Hz, 1H), 1.57-1.48 (m, 2H) 1.46 (s, 9H).
Step E

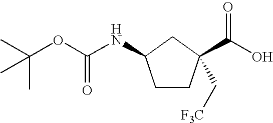

To a mixture of the cis (R,S) product, preparation of which was described in previous step (4.0 g, 12 mmol) in a 1:1:1 solution of tetrahydrofuran/methanol/water (84 mL) was added solid LiOH (2.60 g, 62.0 mmol) and the resulting solution was stirred at 60° C. for 18 h. The mixture was allowed to cool to room temperature and concentrated to remove the organic solvent. The aqueous layer was acidified (pH 4-5) by the slow addition of 6 N hydrochloric acid and the product was extracted with dichloromethane (3×100 mL). The organics were combined, dried over anhydrous sodium sulfate, filtered, and evaporated under reduced pressure to afford Intermediate 6 (3.86 g, 99%) as a yellow oil.
Step F

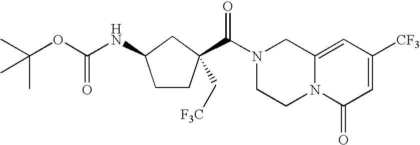

A solution of the acid intermediate, synthesis of which was described in previous step (46 mg, 0.1475 mmol), Intermediate 3 (49 mg, 0.1475 as a trifluoroacetate salt), diisopropyl ethylamine (26 □L, 0.1475 mmol) and catalytic amount of dimethylaminopyridine in dichloromethane (4 mL) was treated with 1-[3-(dimethylamino)propyl]-3-ethylcarbodiimide hydrochloride (EDC, 85 mg, 0.4425 mmol) and stirred at ambient temperature overnight. The reaction mixture was diluted with dichloromethane, and extracted with water (2×). The combined aqueous extracts were backwashed with DCM, the organic extracts were combined, dried (anhydrous sodium sulfate) and the solvent was removed in vacuo. The crude product (131 mg) was purified by preparative TLC to yield 18 mg of the pure product. LC-MS for C₂₂H₂₇F₆N₃O₃ [M+H−BOC]⁺ calculated 412.19, found 412.50.
Step G

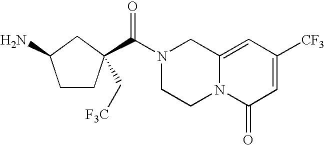

A solution of the BOC-intermediate from the previous step (18 mg, 0.0352 mmol) in dichloromethane (4 mL) was treated with trifluoroacetic acid (1 mL) and stirring was continued at room temperature for 4 hrs. The solvent was removed in vacuo and the crude salt was used in the following reductive amination without any further purification. LC-MS for C₂₂H₂₇F₆N₃O₃ [M+H]⁺ calculated 412.19, found 412.10.

### Intermediate 7

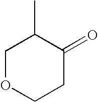

This preparation was performed with six simultaneous batches which were combined for the work-up: A 500 mL flame dried reaction flask was set under static atmosphere of nitrogen and charged with 150 mL of dry THF. Neat diisopropylamine (8.76 mL, 62.5 mmol) was added via syringe and the solution was cooled to −78° C. n-Butyl lithium (25 mL, 2.5M solution in hexanes, 62.5 mmol) was added via syringe to this solution followed by dry HMPA (8.70 mL) and the stirring at cold was continued for 15 minutes. A neat tetrahydro-4H-pyran-4-one (5 g, 50 mmol) was then added via syringe and the anion was allowed to form for 2 hrs at −50° C. Methyl iodide (12.45 mL, 200 mmol) was added and the solution was allowed to warm up to ambient temperature, overnight. The reaction was quenched with a saturated solution of ammonium chloride (50 mL), and all six batches were combined. The crude product was extracted diethyl ether (4×250 mL). The combined organic extracts were concentrated on a Vigreaux column, at ambient pressure. The residue was purified by gradient chromatography using a mixture of ether and pentane mixtures (starting with 10% diethyl ether, final concentration 40%). The fractions containing the pure product were combined, and the solvent was removed using once again a Vigreaux distillation column, at ambient pressure. The pure product (11.05 g, 33%) was obtained by distillation of this residue at ambient pressure, boiling point 169-171° C.

### Intermediate 8

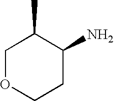 Step A

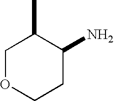

The cis-racemate of 3-methyl-4-amino-tetrahydropyrane was obtained from 3-methyltetrahydropyran-4-one (Intermediate 7) in a procedure analogous to that described in the literature (Allergretti, M., Berdini, V., Cesta, M. C., Curti, R., Nicolini, L., and Topai, A., Tetrahedron Lett., 2001, 42, (25), 4257-9).
Step B

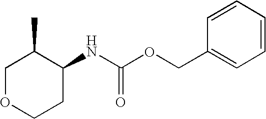

A solution of the amine from the previous step (1.54 g, 10.3 mmol) and diisopropylethylamine (4.46 mL, 25.6 mmol) in dry dichloromethane, under N₂ at ambient temperature, was treated with neat carbobenzoxy chloride (1.61 mL, 11.3 mmol) and the resulting mixture was stirred at room temperature for 2 h. It was diluted with dichloromethane and extracted with 10% aqueous solution of citric acid. The aqueous phase was back extracted with dichloromethane, and the combined organic extracts were washed with saturated aqueous sodium bicarbonate. After drying (anhydrous magnesium sulfate), the solvent was removed in vacuo and column chromatography (Silica gel, ethyl acetate:hexane/2:3) gave 1.8347 g (72%) of the pure product. The respective enantiomers were obtained by chiral HPLC using a ChiralPak AD semi-preparative column. The absolute configuration of the faster eluting isomer (Tr=13.0 minutes, Hexane:EtOH/93:7, 9 mL/min) was shown to be (3R,4S) by both derivatization of the free amine followed by NMR spectroscopy, as well as single crystal X-ray diffraction analysis. ¹H NMR (500 MHz, CDCl₃): 7.47 (bm, 5H), 5.12 (bs, 2H), 4.65 (bd, J=8.7 Hz, 1H), 3.98 (dd, J=11.44, 3.43 Hz, 1H), 3.87 (dd, J=11.4, 4.3 Hz, 1H), 3.45 (m, 2H), 3.08 (t, J=11.40 Hz, 1H), 1.95 (d, J=11.60 Hz, 1H), 1.50 (m, 2H), 0.90 (d, J=6.63 Hz, 3H).
Step C

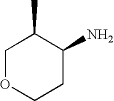

The solution of the CBZ-protected amine from the previous step (284 mg, 1.14 mmol) in ethanol (15 mL) was hydrogenated using 133 mg of Pd/C (10%) under an ambient hydrogen pressure of a balloon for 30 minutes. The catalyst was filtered off, and the solution was concentrated in vacuo to leave 158 mg (91%) of the desired product.

### Intermediate 9

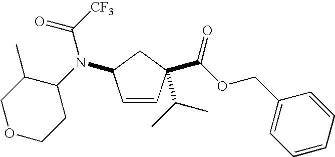 Procedure A
Step A

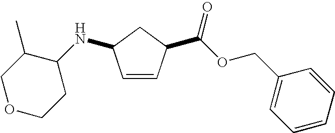

A solution of (1R,4S)-1-amino-4-benzyloxycarbonyl-cyclopent-2-ene hydrochloride (8.94 g, 35.0 mmol), preparation of which was described under Intermediate 4, Steps A —C, the ketone Intermediate 8 (7.04 g, 69.91 mmol) in 50 mL of dichloroethane was treated with crushed 4 A molecular sieves (10 g), diisopropylethylamine (6.1 mL, 35 mmol) and sodium triacetoxyborohydride (29.5 g, 140 mmol) and the reaction mixture was stirred at room temperature for 36 hrs. Dichloromethane (300 mL) was added, and the reaction was quenched with saturated solution of sodium bicarbonate (100 mL). The aqueous layer was extracted with DCM three more times, the combined organic phases were dried, filtered, and the solvent was removed in vacuo. This crude mixture of isomers (15.0711 g), was used in the next reaction step without further purification.
Step B

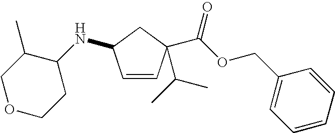

A flame-dried 500 mL reaction flask was charged with potassium hexamethyldisilazane (9.07 g, 45.50 mmol) and set under static atmosphere of nitrogen. Tetrahydrofuran (300 mL) was added via canula and the solution was cooled to −78° C. A tetrahydrofuran (20 mL) solution of the crude ester (15.07 g) from previous step was then added via syringe and stirring at cold was continued for 90 minutes. Neat isopropyl iodide (10.48 mL, 105.0 mmol) was then added and the stirring at −78° C. was continued for 30 minutes, than allowed to warm up to −40° C. The reaction mixture was poured onto a saturated solution of ammonium chloride (200 mL) and the crude product was extracted with dichloromethane (4×150 mL). The combined organic phases were dried with anhydrous sodium sulfate, filtered, and the solvent was removed in vacuo to afford 11.74 g of the crude isomeric mixture. No purification was attempted at this point, and the mixture was used in the subsequent step as obtained.
Step C

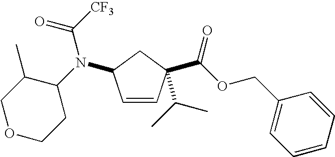

A solution of the anine, preparation of which was described in the previous step (11.74 g, max. 32.9 mmol) and diisopropylethylamine (28.6 mL, 164.2 mmol) in dichloromethane (100 mL) was treated at 0° C. with trifluoroacetic anhydride (13.8 mL, 65.8 mL). The cooling bath was removed, and the stirring was continued for another 2 hrs. The reaction was quenched with saturated aqueous sodium bicarbonate (100 mL), and the crude product was extracted with dichloromethane (4×100 mL). The combined organic extracts were dried (anhydrous sodium sulfate) and the solvent was removed in vacuo. The crude product was purified by gradient chromatography (0% to 30%) of ethyl acetate in hexanes to afford 9.93 g (66%, three steps). Under these conditions, the major cyclopentane-cis-isomer could be successfully separated from the minor trans isomer. This product was still a mixture of isomers at the tetrahydropyrane ring. LC-MS for C₂₄H₃₀F₃NO₄ [M+H]⁺ calculated 424.21, found 454.10.

Procedure B

Step A

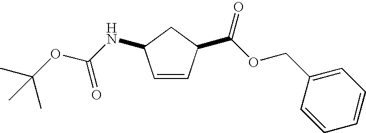

A solution of (1R,4S)-4-tert-butyoxycarbonylamino-cyclopent-2-enecarboxylic acid (15.0 g, 66.0 mmol) a preparation of which was described under Intermediate 4, Step A, benzyl bromide (7.85 g, 66 mmol) in DMF (30 mL) and potassium carbonate (13.7 g, 99 mmol) were vigorously stirred at room temperature for 2 hrs. The reaction mixture was diluted with hexanes (200 mL) and quenched with water (100 mL). The organic phase was separated, and the extraction repeated 3 more times. The combined organic phases were dried (anhydrous sodium sulfate) and the solvent was removed in vacuo. The crude product was further purified by gradient chromatography, using a mixture of ethyl acetate and hexanes as an eluent. The concentration of the ethyl acetate was gradually increased from 0% to the final 40%. This way 19.39 g (93%) of the desired product was obtained. LC-MS for C₁₈H₂₃NO₄ [M+Na]⁺ calculated 340.20, found 340.10. ¹H NMR (500 MHz, CDCl₃) δ 7.35 to 7.40 (m, 5H), 5.90 (bs, 2H), 5.16 (s, 2H), 3.54 (dd, J=8.7, 4.4 Hz, 1H), 2.53 (ddd, J=14.0, 8.5, 8.5 Hz, 1H), 1.92 (ddd, J=14.0, 4.1, 4.1 Hz, 1H), 1.46 (s, 9H). ¹³C NMR (500 MHz, CDCl₃) δ 155.1, 135.6, 134.9, 131.0, 128.6, 128.3, 128.0, 66.7, 55.7, 49.3, 34.5, 28.4.
Step B

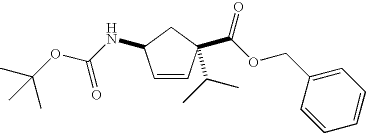

A flame dried 500 mL flask was charged with lithium hexamethyldisilazane (10.9 g, 65.18 mmol) and set under static atmosphere of nitrogen. THF (40 mL) was added via canula and the solution was cooled to −78° C. A solution of the ester from the previous step (9.40 g, 29.63 mmol) in THF (20 mL) was then added via syringe and the anion was allowed to form for 30 minutes. Neat isopropyliodide (3.55 mL, 35.56 mmol) was then added via syringe, and the reaction mixture was stirred at −40° C. for 30 minutes, than at −15° C. for 3 hrs. The reaction was quenched with 10% aqueous solution of citric acid, and the product was extracted with diethyl ether (3×150 mL). The combined solvents were dried (anhydrous sodium sulfate) and the solvent was removed in vacuo. The crude product was further purified by gradient chromatography, using ethyl acetate and hexane mixture as an eluent. During the purification, the concentration of ethyl acetate was gradually increased from 0% to 35%. In this manner 4.1211 g (39%) of pure cis-isomer was obtained. LC-MS for C₂₁H₂₉NO₄ [M+Na]⁺ calculated 382.21, found 382.25. ¹H NMR (500 MHz, CDCl₃) δ 7.36 (bm, 5H), 5.80 (m, 2H), 5.13 (ABq, J=12.3 Hz, 2H), 2.30 (m, 2H), 2.03 (m, 1H), 1.46 (s, 9H), 0.83 (d, J=6.6 Hz, 3H), 0.8 (d, J=6.6, 3H).
Step C

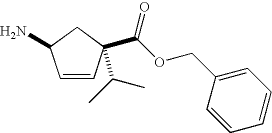

A solution of the BOC-protected amine intermediate from the previous step (4.66 g, 13.0 mmol) in dichloromethane (4 mL) was treated with trifluoroacetic acid (2.0 mL), and stirred at room temperature for 6 hrs. The solvent was removed in vacuo, and the obtained amine trifluoroacetamide (6.1 g) was used in subsequent step without any further purification. LC-MS for C₁₆H₂₁NO₂ [M+H]⁺ calculated 260.16, found 260.15.
Step D

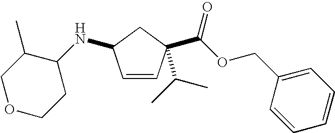

A mixture of the amine from the previous step (6.12 g, 12.96 mmol), Intermediate 7 (2.76 g, 24.18 mmol), diisopropylethylamine (2.26 mL, 12.96 mmol), crushed 4 A molecular sieves (5.0 g) and sodium triacetoxyborohydride (8.25 g, 38.88 mmol) in dichloromethane (40 mL) was stirred at ambient temperature overnight. It was poured onto saturated solution of sodium bicarbonate and the crude product was extracted with dichloromethane. The combined extracts were dried with anhydrous sodium sulfate, filtered, and the solvent was removed in vacuo to yield 4.66 g (100%, 2 steps) of the desired product in a form of an isomeric mixture. It was used in the subsequent step without further purification. LC-MS for C₂₂H₃₁NO₃ [M+H]⁺ calculated 358.23, found 358.25.
Step E

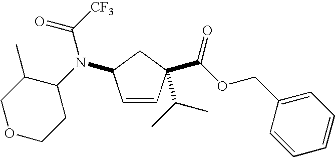

A solution of the amine, preparation of which was described in the previous step (2.51 g, 7.04 mmol) and diisopropylethylamine (6.13 mL, 35.21 mmol) in dichloromethane (30 mL) was cooled to 0° C. and treated with trifluoroacetic anhydride (1.98 mL, 14.08 mmol). The reaction mixture was stirred at ambient temperature for 30 minus, and was quenched with 10% aqueous solution of citric acid (50 mL). The product was extracted with dichloromethane, the combined organic extracts were dried with anhydrous sodium sulfate, and the solvent was removed in vacuo. This crude product (3.81 g) was further purified by gradient chromatography (ethyl acetate-hexanes, 0 to 50% of ethyl acetate) to afford 2.49 g (78%) of the desired product as a mixture of tetrahydropyrane-derived isomers. LC-MS for C₂₄H₃₀F₃NO₄ [M+H]⁺ calculated 424.21, found 454.10.

### Intermediate 10

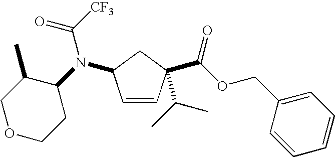

Intermediate 10, as a single isomer of indicated absolute stereochemistry, was obtained from Intermediate 9 by means of chromatographic separation using a Chiralpak AD semi-preparative column as the faster eluting major isomer. The eluent composed of a mixture of hexanes and ethyl alcohol in a ratio of 4:1, the employed flow rate was 9 mL a minute. The respective retention time on an analogous analytical column (flow rate of 1.0 mL/min) was 8.58 minutes. NMR (500 MHz, CDCl₃) δ 7.38 bs, 5H, 6.01 (dd, J=5.5, 2.1 Hz, 1H), 5.76, bs 1H, 5.21 s, 2H, 5.0 s, 1H, 3.85 (d, J=8.7 Hz, 1H), 3.60 (d, J=11.2 Hz, 1H), 2.30 (m, 3H), 1.74 bs, 1H), 1.20 m, 5H, 0.84 m, 6H.

### Intermediate 11

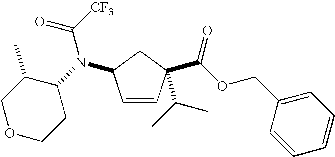

Intermediate 11 was obtained from the isomeric mixture preparation of which was detailed under Intermediate 9 using conditions described for isolation of Intermediate 10 as the slower eluting major isomer. The respective retention time of this isomer on a analytical Chiralpak AD column was 9.55 minutes, maintaining a flow rate of 1.0 mL/min.

### Intermediate 12

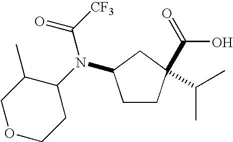

A solution of the benzyl ester Intermediate 9 (9.93 g, 21.89 mmol) in ethanol (100 mL) and palladium on carbon (520 mg, 10%) was hydrogenated in a Parr shaker at 50 psi for 4 hr. The catalyst was filtered off, and the solvent was removed in vacuo to yield the desired product (8.21 g, quantitative) as a mixture of isomers at the tetrahydropyrane ring. LC-MS for C₁₇H₂₆F₃NO₄ [M+H]⁺ calculated 366.18, found 366.20.

### Intermediate 13

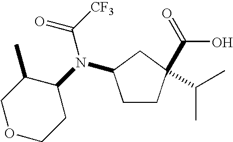 Procedure A

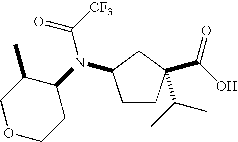

A solution of Intermediate 10 (650 mg), 1.43 mmol in ethyl alcohol (50 mL) was hydrogenated in a Parr shaker at 50 psi pressure in a presence of Pd/C (10%, 200 mg) for 4 hrs. The catalyst was filtered off, and the solvent was evaporated to dryness to leave a white solid (512 mg, 98%). LC-MS for C₁₇H₂₇F₃NO₄ [M+H]⁺ calculated 366.18, found 366.05.

Procedure B

Step A

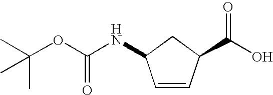

A mixture of (1R,4S)-4-amino-cyclopen-2-ene carboxylic acid (130 g, 1.0 mol), water (250 mL), sodium bicarbonate (170 g, 2.0 mol) and tetrahydrofuran (750 mL) was stirred for 30 min, then solid di-tert-butyl dicarbonate (230 g, 1.05 mol) was added. The mixture was stirred over the weekend, filtered to remove the insoluble material, evaporated to remove the tetrahydrofuran, and cooled to 0° C. To the residue was added 2 N aqueous HCl until the pH reached 3 (˜500 mL). The resulting precipitate was collected by filtration and washed with water and dried under vacuum overnight. The desired acid was obtained as a white solid (230 g, 100%). ¹H NMR (400 MHz, CD₃OD): δ 5.95 (m, 1H), 5.79 (m, 1H), 4.80 (br s, 1H), 3.45 (m, 1H), 2.50 (m, 1H), 1.79 (m, 1H), 1.44 (s, 9H).
Step B

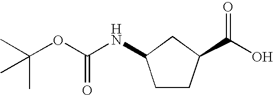

The acid prepared in Step A (230 g, 1.0 mol) and 10% Pd/C (5.0 g) in 500 mL of methanol was placed on a Parr apparatus and hydrogenated under 50 psi of hydrogen for 1 h. The catalyst was removed by filtration and the filtrate was evaporated. The residue was dissolved in dichloromethane and dried over anhydrous sodium sulfate. After filtration, the filtrate was evaporated and dried under vacuum. The title compound was obtained as a light yellow solid (230 g, 99%). LC-MS for C₁₁H₁₉NO₄ calculated 229, found [M+H]⁺ 230.
Step C

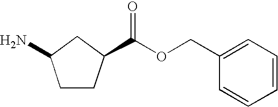

To a mechanically stirred solution of the acid prepared in Step B, Intermediate 9 (230 g, 1.00 mol) in 500 mL of N,N-dimethylformamide was added solid potassium carbonate (210 g, 1.5 mol). The resulting mixture was stirred for 20 min and neat benzyl bromide (120 mL, 1.0 mol) was added in one portion. An exothermic reaction was observed. After being stirred for 3 h at room temperature, the entire mixture was poured into an ice-water mixture (1000 mL). The crude product was extracted out with ether (2×800 mL). The combined ether layers were washed with water, dried over sodium sulfate, filtered and evaporated to offer a yellow solid. This solid was mixed with 4 N HCl in dioxane (400 mL), stirred overnight and condensed. The resulting solid was collected by filtration, washed with ether and dried under vacuum. The title product was obtained as a hydrochloride salt (140 g, 55%). ¹H NMR (400 MHz, CD₃OD): δ 5.15 (s, 2H), 3.65 (m, 1H), 3.02 (q, J=8 Hz, 1H), 2.50 (m, 1H), 2.15 (m, 1H), 2.05 (m, 2H), 1.90 (m, 1H), 1.75 (m, 1H).
Step D

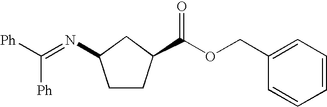

The amino benzyl ester HCl salt prepared in Step C, (130 g, 0.50 mol) was suspended in 500 mL of dichloromethane. Benzophenone imine (91 g, 0.50 mol) was added. The resulting mixture was stirred overnight, and filtered to remove the inorganic salt. The filtrate was washed with water and brine, dried over sodium sulfate, and evaporated. The residue was dissolved in 200 mL of toluene, and evaporated. This procedure was repeated once more. The title compound (178 g) was obtained as a brown oil which was used in the next step without further purification. ¹H NMR (400 MHz, CDCl₃): δ 1.80 (m, 1H), 1.95 (m, 2H), 2.15 (m, 2H), 2.50 (m, 1H), 2.89 (m, 1H), 3.61 (m, 1H), 5.20 (s, 2H), 7.18 (d, 2H), 7.38 (m, 8H), 7.47 (m, 3H), 7.64 (d, 2H).
Step E:

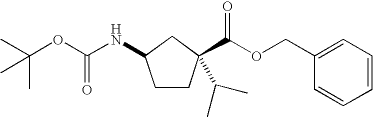

The Schiff base benzyl ester from Step D, (76.6 g, 200 mmol) in 300 mL of tetrahydrofuran was cooled to −78° C. under nitrogen. While stirring, a solution of lithium diisopropylamide (2.0 M, 110 mL, 220 mmol) in heptane was added over 20 min. The mixture was stirred for 30 min at −78° C., then a solution of 68 mL of isopropyl iodide (440 mmol) in 50 mL of tetrahydrofuran was added, and the mixture was allowed to stir for 30 min. The reaction temperature was raised to 0° C. by removing the cooling bath. After being stirred for 2 h, the entire mixture was evaporated to remove the tetrahydrofuran. The residue was dissolved in ether (1000 mL), washed with water and brine, dried over sodium sulfate, and evaporated. The crude product was dissolved in 500 mL of tetrahydrofuran, mixed with 400 mL of aqueous 1 N HCl, stirred for 1 h, and evaporated to remove tetrahydrofuran at 50° C. The aqueous solution was extracted with hexanes (3×), made alkaline with saturated aqueous sodium carbonate (pH>9) and treated with a solution of di-tert-butyl dicarbonate (53 g) in 500 mL of dichloromethane. The resulting reaction mixture was stirred for 30 min. The organic phase was separated and the aqueous phase was extracted with dichloromethane (3×). The combined organic phases were dried over sodium sulfate and evaporated. The residue was purified by flash chromatography (silica gel, 10% ethyl acetate/hexanes) to yield a mixture of the title compound as a mixture of cis and trans isomers (˜1:1, 24 g). Further purification by MPLC (8% ethyl acetate/hexanes) afforded the single desired cis isomer (fast-eluted, 7.3 g) and the undesired trans isomer (slow-eluted). ESI-MS calculated for C₂₁H₃₁NO₄: 361; Found: [M+H]⁺ 362. ¹H NMR (500 MHz, CDCl₃): δ 7.36 (m, 5H), 5.14 (s, 2H), 4.77 (m, 1H), 4.01 (d, J=5.0 Hz, 1H), 2.17 (m, 1H), 1.99-1.53 (m, 5H), 1.42 (m, 9H), 0.85 (d, J=7.0 Hz, 6H).
Step F

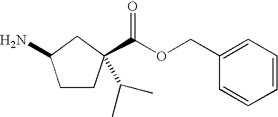

The BOC-protected amine (15.73 g, 43.52 mmol) in dichloromethane (100 mL) was treated at room temperature with trifluoroacetic acid (25 mL) and stirred at ambient temperature 2 hrs. The solvent was removed in vacuo, the residue was co-distilled two more times with toluene. Removal of the solvent at reduced pressure gave the pure desired amine in a form of a hydrochloride salt.
Step G

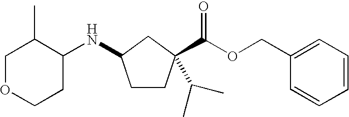

A solution of the amine hydrochloride, preparation of which was described in the previous step (1.82 g, 6.11 mmol), ketone Intermediate 7 (697 mg, 6.11 mmol), crushed 4A molecular sieves (3.2 g), diisopropylethylamine (1.0 mL, 6.11 mmol) in anhydrous dichloromethane was treated with sodium triacetoxyborohydride (3.9 g, 18.33 mmol) and stirred at room temperature overnight. The reaction was quenched with addition of aqueous saturated solution of sodium bicarbonate (100 mL) and extracted with dichloromethane (4×100 mL). The combined organic extracts were back-washed with brine, dried with anhydrous sodium sulfate and the solvent was removed in vacuo. to yield 2.54 g of the crude product, which was used in the next reaction step without additional purification. ESI-MS calculated for C₂₂H₃₃NO₃: 359.25; Found: [M+H]⁺ 360.25.
Step H

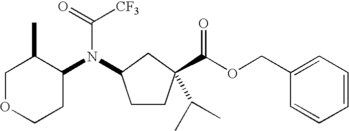

The crude product (2.54 g) was dissolved in dry dichloromethane (40 mL), diisopropylethylamine (3.7 mL, 21.21 mmol) was added and the mixture was cooled to 0° C. To this cold solution was added neat trifluoroacetylanhydride (1.20 mL, 8.49 mmol) and the reaction mixture was stirred at cold for 30 minutes. It was poured onto a 1N solution of HCl, the organic layer was separated, and the aqueous was extracted with dichloromethane 3 more times. The combined organic extracts were dried and the solvent was removed in vacuo. The particular isomer of the desired cis-absolute stereochemistry was obtained by carefully performed gradient flash chromatography on silicagel, using a mixture of ethyl acetate and hexanes in which the concentration of the ethyl acetate was gradually increased from 0% at the beginning to the final 40% at the end of the run. Under these conditions the desired cis-isomer (910 mg), eluted first.
Step I

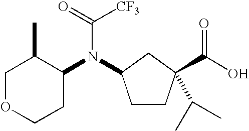

A solution of the benzyl ester intermediate, preparation of which was described in the previous step (3.20 g, 8.90 mmol) and palladium on charcoal (520 mg, 10%) in ethyl alcohol (250 mL) was hydrogenated at ambient pressure for 2 hrs. The catalyst was filtered off, and the solvent was removed in vacuo. to yield 2.27 g (70%) of the desired acid.

### Intermediate 14

Step A 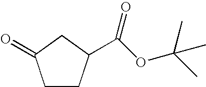
Procedure A

A solution of 3-oxo-cyclopentane carboxylic acid (Stetter, H., Kuhlmann, H. Liebigs Ann. Chem., 1979, 7, 944-9) (5.72 g, 44.6 mmol) in dichloromethane (30 mL) was treated with N,N′-diisopropyl-O-tert-butyl-iso-urea (21.2 mL, 89.3 mmol) and the reaction mixture was stirred at ambient temperature overnight. The precipitated N,N′-diisopropyl urea was filtered off, the filtrate concentrated in vacuo and the residue was purified by distillation (bp: 125-129° C. @ 18 mmHg) to yield 4.74 g (58%) of the pure product. ¹H NMR (500 MHz, CDCl₃): δ 3.02 (p, J=7.8 Hz, 1H), 2.05-2.50 (m, 6H), 1.45 (s, 9H). ¹³C NMR (125 MHz, CDCl₃): δ 217.00, 173.47, 80.99, 41.88, 41.14, 27.94, 26.57.

Procedure B

A 2 L RBF was charged with anhydrous magnesium sulfate (113 g, 940 mmol) and dichloromethane (940 mL) was added. While stirring, the suspension was treated with concentrated sulfuric acid (12.5 mL, 235 mmol), followed by, in 15 minutes by 3-oxo-cyclopentane carboxylic acid (30.1 g, 235 mmol). After stirring for 15 minutes, tert-butanol (87 g, 1.2 mol) was added. The reaction vessel was closed with a stopper to aid retention of isobutylene, and stirred at ambient temperature for 72 hours. The solid was filtered off through a plug of celite, volume of the filtrate was reduced to approximately 500 mL, and washed with saturated solution of sodium bicarbonate (2×150 mL). The organic phase was dried with anhydrous magnesium sulfate, filtered, and the solvent was removed by distillation at reduced pressure (180 mmHg). The crude product was purified by distillation to yield 39.12 g (90%) of pure product.
Step B

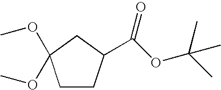

A solution of tert-Butyl 3-oxocyclopentane carboxylate (11.54 g, 62.64 mmol) in dichloromethane (200 mL) was treated with trimethyl orthoformate (41.4 mL, 251 mmol) in the presence of p-toluenesulfonic acid (400 mg) and stirred at room temperature for 48 hours. The dark reaction mixture was poured onto saturated solution of sodium bicarbonate, and the crude product was extracted with dichloromethane. The combined organic extracts were dried with anhydrous magnesium sulfate, the solvent was removed in vacuo, and the crude product was purified by distillation (bp.: 104° C. @ 4 mmHg) to yield 12.32 g (85%) of the desired product. ¹H NMR (500 MHz, CDCl₃): δ 3.21 (s, 3H), 3.20 (s, 3H), 2.80 (m, 1H), 2.10 to 1.80 (bm, 6H), 1.46 (s, 9H). ¹³C NMR (125 MHz, CDCl₃): δ 174.9, 111.2, 80.3, 67.8, 49.2, 42.5, 37.4, 33.8, 28.3, 22.0.
Step C:

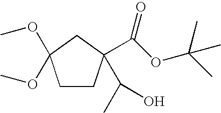

A flame dried 500 mL round bottom flask was charged with 100 mL of dry THF, and then, set under nitrogen and cooled to −78° C. using an acetone/dry ice bath. Diisopropylamine (7.9 mL, 56 mmol) was added to the cooled solvent via syringe followed by the slow addition of 2.5 M n-butyllithium in hexane (22.6 mL, 56.45 mmol). After 5 minutes stirring, the acetal (described in Step B, Intermediate 6, 10.0 g, 43.4 mmol) in 50 mL of THF was added dropwise via syringe and the resulting mixture stirred at −78° C. for 2 hours. Acetylaldehyde (7.3 mL, 130 mmol) was then added dropwise via syringe and the resulting mixture was stirred for 2 h at −78° C. The reaction was quenched by pouring the mixture into a solution of 10% citric acid (300 mL) and then extracting with dichloromethane (2×150 mL). The organics were combined, dried over anhydrous magnesium sulfate, filtered, and evaporated under reduced pressure. During the reaction or work-up some of the acetal was hydrolyzed to the ketone, therefore, the crude mixture was taken onto the next step without purification.
Step D

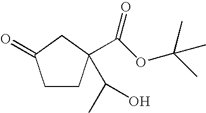

The crude intermediate (described in Step C, Intermediate 6, 56.45 mmol assumed 100% conversion for Step C) was treated with a solution of 10% trifluoroacetic acid in dichloromethane and the resulting mixture stirred overnight at room temperature. The reaction was concentrated in vacuo, then diluted with water, and extracted with dichloromethane. The organics were combined, dried over anhydrous magnesium sulfate, filtered, and evaporated under reduced pressure to afforded 8.04 g (83%) of the crude product that was used without further purification.
Step E

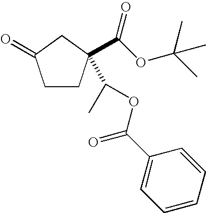

A solution of the alcohol from the previous step (2.86 g, 12.529 mmol), benzoic acid (1.54 g, 13.782 mmol), DMAP (300 mg) in dichloromethane (50 mL) was treated with EDC, and the reaction mixture was stirred at ambient temperature overnight. The reaction was quenched with water (50 mL) and the crude product was extracted into dichloromethane (4×50 mL). The combined organic phases were dried with anhydrous magnesium sulfate, filtered, and the solvent was removed in vacuo (4.92 g). The respective erhythro- and threo-diastereoisomeric pairs could be easily separated using gradient column chromatography using a mixture of ethyl acetate and hexane as eluent. The concentration of ethyl acetate was gradually increased from 0% at the beginning of the separation to 50% at the end. In this fashion, 1.309 g (32%) of the higher eluting and 1.322 g of the lower eluting diastereoisomeric pair could be obtained. The lower eluting diastereoisomeric pair was further separated into its components using a semipreparative chiral column chromatography: Chiralcel OD, hexane+ethyl alcohol (98:2), flow rate of 9.0 mL/minute. Under these conditions, the active isomer eluted second, and 518 mg of pure product was obtained. Its retention time under analogous analytical conditions (1.0 mL/minute flow rate) the first isomer eluted with a retention time of 10.01 minutes, while the desired, second isomer eluted with a retention time of 11.39 minutes. LC-MS for C₁₉H₂₄O₅ [M+H+Na]⁺ calculated 355.15, found 355.10.
Step F

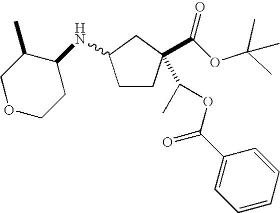

A solution of the ketone from the previous step (500 mg, 1.5043 mmol), Intermediate 8 (228 mg as a hydrochloride salt, 1.5043 mmol), crushed 4A molecular sieves (2.5 g), diisopropylethylamine (263 μL, 1.5043 mmol) in dichloromethane (10 mL) was treated with sodium triacetoxyborohydride (956 mg, 4.51 mmol) and stirred at ambient temperature for 72 hrs. The reaction mixture was then poured onto saturated aqueous solution of sodium bicarbonate (50 mL) and the product was extracted with dichloromethane (4×50 mL). The combined organic phases were dried (anhydrous magnesium sulfate), filtered, and the solvent was removed in vacuo. The residue (590 mg) was purified by preparative TLC (dichloromethane+methanol+ammonium hydroxide/90+9+1) to yield 538 mg of desired product as a mixture of the cyclopentane-derived cis- and trans-isomers. This mixture was used in the next step without further purification. LC-MS for C₂₅H₃₇NO₅ [M+H]⁺ calculated 432.27, found 432.20.
Step G

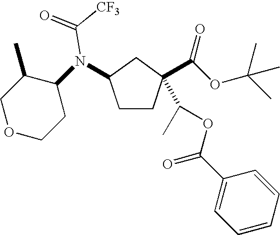

A solution of the amine from the previous step (478 mg, 1.1 mmol), triethylamine (460 mg, 3.3 mmol) in dichloromethane (6 mL) was cooled to 0° C. and while stirring, trifluoroacetic anhydride (253 μL, 1.66 mmol) was added via syringe. Stirring at 0° C. was continued for another 30 minutes, and the reaction was quenched by pouring onto saturated aqueous solution of sodium bicarbonate (30 mL). The product was extracted with dichloromethane (4×30 mL), combined organic extracts were dried (anhydrous magnesium sulfate) and the solvent was removed under reduced pressure. The residue (676 mg) was purified by gradient chromatography (ethyl acetate:hexanes/0 to 60% of ethyl acetate) to yield 460 mg of the desired product as a mixture of the respective cyclopentane derived cis- and trans-isomers. The respective cis-isomer was obtained by semipreparative dhiral chromatography, using Chiralcel OD column, and a mixture of hexane and ethyl alcohol (98:2) as an eluent. Under these conditions, the trans-isomer elutes first (the respective retention time on an analytical column, flow rate of 1.0 mL/minute, was 6.36 minutes (38%) and the cis-isomer eluting second, with an analytical retention time of 9.34 minutes (68%). This separation yielded 240 mg of desired product in a form of a single isomer. LC-MS for C₂₃H₂₈F₃NO₆ [M+H−tBuO—]⁺ calculated 454.18, found 432.454.10.
Step H

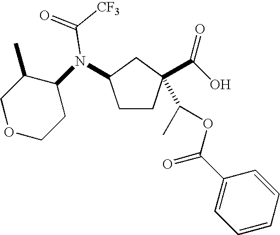

A solution of the ester from the previous step (159 mg, 0.3016 mmol) in dichloromethane (4 mL) was treated with trifluoroacetic acid (2 mL) and stirred at room temperature for 90 minutes. The solvent was removed in vacuo, and the crude product was used in the subsequent steps without further purification. LC-MS for C₂₃H₂₈F₃NO₆ [M−OH]⁺ calculated 454.18, found 432.454.10.

### Intermediate 15

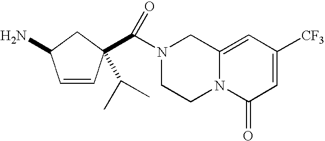 Step A

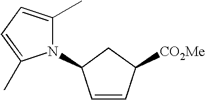

The solid aminocyclopentane methyl ester salt (1.076 kg, 6.059 mol) was dissolved in MeOH (3 L, 2M) at 20° C. under nitrogen. Diisopropylethylamine (DIEA, 0.78 kg, 6.059 mol) was added followed by acetonyl acetone (0.711 kg, 6.241 mol). The batch had an exotherm increasing the temperature to 32-35° C. The reaction mixture was then aged at 25° C. for 16 h. The batch was diluted with IPAc (9-10 L) and washed with 10% NH4Cl (2×3 L) and 5% brine (2×3 L). The IPAc batch was dried over sodium sulfate, filtered, and concentrated to an oil. THF (3 L) was used as a flush and the batch was again concentrated to an oil. The air-sensitive pyrrole-protected aminocyclopentane carboxylate (1189 g, 92% yield) was stored at 5-7° C. under nitrogen until the alkylation step was run.
Step B

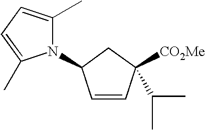

The pyrrole methyl ester (1189 g) dissolved in THF (1.2 L) was added dropwise over 40 min to 1 M lithium hexamethyldisilazide (LHMDS) in THF (8.65 L, 8.650 mol) at −20° C. The batch was aged for 30 min and 2-iodopropane was added over 1 h. The batch was aged for 1 h, then allowed to warm to 20° C. over 2 h and aged at 20° C. for 1-2 h until complete by HPLC (<0.5% starting material). The batch was quenched into 6% NH₄Cl solution (10 L). IPAc (20 L) was charged and the layers were separated. The organic layer was washed with 6% aq NH4Cl (10 L), 5% brine (2×10 L), and concentrated to an oil. The air-sensitive alkylated pyrrole methyl ester (1419 g, 98% yield) was stored at 5-7° C. under nitrogen until saponified.
Step C

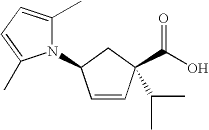

The alkylated pyrrole methyl ester (1.38 kg, 5.197 mol) was dissolved in MeOH (7.7 L). DI water (2.5 L) was added followed by ION NaOH (2.08 L, 20.786 mol). The batch was then heated to 65° C. for 16 h. The batch was cooled to 10° C. The product was crystallized by adjusting the pH to 4.5 with concd HCl. The slurry was aged for 1 h and DI water (15 L) was charged to the batch. The slurry was aged 18 h at 20-25° C. The solids were filtered, washed with 10% MeOH/DI water and dried in a vacuum oven (40-50° C., 25-26″ Hg) to provide the alkylated pyrrole cyclopentene acid (1223 g, 95% yield).
Step D

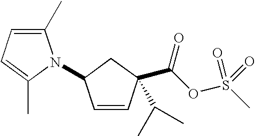

A solution of the acid from the previous step (1.50 g, 6.05 mmol), diisopropylethylamine (2.11 mL, 12.1 mmol) in THF (20 mL) was cooled to 0° C. and with stirring, neat methanesulfonyl chloride (468 μL, 6.05 mmol) was added. The cooling bath was removed, and stirring at rt was continued for an additional 45 minutes. A small sample of the reaction mixture was quenched with methyl alcohol, and a subsequent HPLC analysis confirmed a complete conversion to the respective methyl ester. LC-MS for C₁₆H₂₃NO₂ [M+H]⁺ (Methyl Ester) calculated 262.17, found 262.10. This solution of the mixed anhydride was used in the amide formation step without any further delay.
Step E

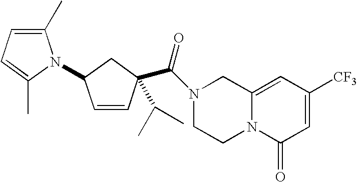

A solution of the Intermediate 3 (hydrochloride, 1.54 g, 6.05 mmol) and diisopropylethylamine (2.11 mL, 12.10 mmol) in tetrahydrofurane (10 mL) was cooled to 0° C. and the solution of the mixed anhydride from the previous step was added via syringe. The cooling bath was removed, and stirring at room temperature was continued for one hour. At this point, no more active mixed anhydride could be detected by the above described methanol quench, and the appearance of a new peak, corresponding to the desired product was observed. Water (50 mL) was added, and the product was extracted with dichloromethane (4×50 mL). The combined organic extracts were dried, and the solvent was removed in vacuo. This crude product (3.76 g) was further purified by gradient chromatography (silica gel, ethyl acetate/hexanes, 0% to 100% of ethyl acetate) to afford 2.51 g (93%) of the desired product. LC-MS for C₂₄H₂₈N₃O₂ [M+H]⁺ calculated 448.21, found 448.20. ¹H NMR (500 MHz, CDCl₃): 6.80 (s, 1H), 6.26 (s, 1H), 6.20 (dd, J=5.7, 2.5 Hz, 1H), 6.02 (dd, J=6.0, 2.1 Hz, 1H), 5.75 (s, 2H), 5.31 (m, 1H), 4.81 (bs, 1H), 4.65 (bs, 1H), 4.24 (m, 2H), 4.13 (m, 2H), 3.93 (m, 2H), 2.68 (dd, J=13.5, 8.7 Hz, 1H), 2.20 (bm, 7H), 2.05 (s, 1H), 1.28 ( )m, 1H), 0.96 (d, J=6.64, 3H), 0.94 (d, J=6.9 Hz, 3H).
Step F

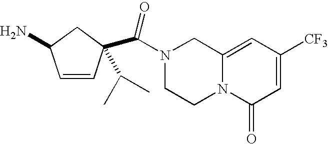

A solution of the pyrrole from the previous step (2.51 g, 5.61 mmol) in ethanol (80 mL) was treated with hydroxylamine hydrochloride (7.8 g, 112.2 mmol), followed by an aqueous solution of sodium hydroxide (11.2 mL, 5N, aq.) and the reaction mixture was stirred at gentle reflux overnight. The solvent was removed in vacuo, the residue was picked up into 50 mL of aqueous sodium bicarbonate, and the product was extracted with a mixture of chloroform and isopropyl alcohol (85:15, 6×100 mL). The combined extracts were dried and the solvent was removed under reduced pressure. This product was used in the subsequent reductive amination step without any further purification.

### EXAMPLE 1

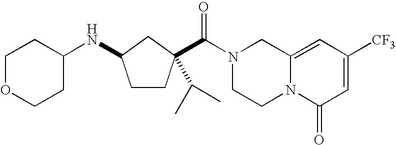 Step A

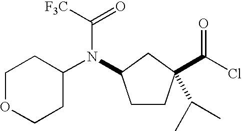

A solution of the acid Intermediate 4 (289 mg, 0.8225 mmol) in anhydrous dichloromethane (6 mL) was cooled to 0° C. and neat oxalyl chloride (215 μL, 2.47 mmol) was added via syringe, followed by 3 drops of anhydrous DMF. The cooling bath was removed, and the reaction mixture was stirred at room temperature for 2 hrs. The solvent was removed in vacuo, and the residue was distilled using a kugelrohr apparatus (250° C. @ 0.01 mmHg) to yield 303 mg (100%) of the unstable chloride, which was reacted in the next reaction step without any further delay. The sample was analyzed after a methanol quench: LC-MS for C₁₇H₂₆F₃NO₄(methyl ester) [M+H]⁺ calculated 365.18, found 366.20.
Step B

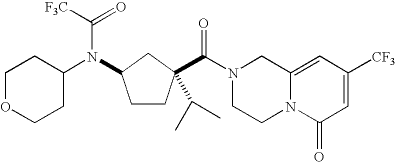

A suspension of the hydrochloride salt of Intermediate 3 (142 mg, 0.5561 mmol) in anhydrous dichloromethane (8 mL) was treated with diisopropyl ethylamine (773 μL, 4.44 mmol), and cooled to 0° C. While stirring, a solution of the acyl chloride, synthesis of which was described in Step A (302 mg, 0.8166 mmol) in dichloromethane (8 mL) was added, via syringe. A reaction mixture was stirred at room temperature for 30 minutes, diluted with dichloromethane (50 mL) and poured onto a saturated aqueous solution of sodium bicarbonate. The organic layer was separated and washed with a solution containing 10% citric acid and 3% of L-ascorbic acid. The organic phase was dried, and the solvent was removed in vacuo to yield the desired product (302 mg, 100%) of satisfactory purity. LC-MS for C₂₅H₃₁F₆N₃O₄ [M+H]⁺ calculated 551.22, found 366.20.
Step C

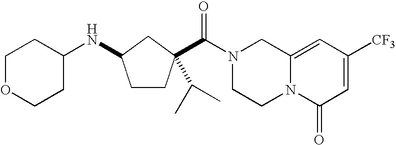

A solution of the amide (150 mg, 0.278 mmol) from the previous step in ethanol (12 mL) was treated with sodium borohydride (210 mg, 5.56 mmol) in several portions in course of 2 hrs. The solvent was evaporated to dryness, the residue was treated with a aq. saturated solution of sodium bicarbonate (20 mL) and the crude product was extracted with chloroform. The combined organic phases were dried with anhydrous sodium sulfate, the solvent was removed in vacuo and the residue (135.6 mg) was purified by preparative TLC using a mixture of ethyl acetate, ethanol and ammonium hydroxide (90:8:2) as an eluent to afford the pure desired product (60.4 mg 47%). LC-MS for C₂₃H₃₂F₃N₃O₃ [M+H]⁺ calculated 456.24, found 456.20.

### EXAMPLE 2

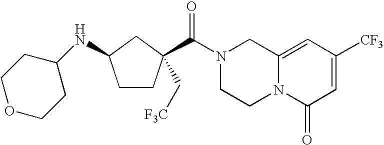

A solution of Intermediate 6 (28 mg, 0.0352 mmol), tetrahydropyran-4-one (10.6 mg, 0.106 mmol) diisopropylethyl amine (6.1 μL) in 4 mL of dichloromethane was treated with crushed 4A molecular sieves (170 mg) and sodium triacetoxyborohydride (37 mg, 0.176 mmol) and stirred at ambient temperature overnight. The reaction was quenched with saturated sodium bicarbonate, and the crude product was extracted with dichloromethane. The combined organic extracts were dried with anhydrous sodium sulfate, filtered and the solvent was removed in vacuo. The remaining crude product (47.5 mg) was further purified by preparative TLC using a mixture of ethyl acetate/ethanol/ammonium hydroxide (90:8 2) as an eluent. In this way, 22 mg of pure product were obtained. LC-MS for C₂₂H₂₇F₆N₃O₃ [M+H]⁺ calculated 496.20, found 496.10.

### EXAMPLE 3

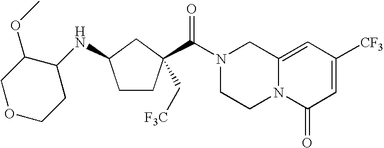

Starting from Intermediate 6 (85 mg, 0.1171 mmol) and a racemic form of Intermediate 5 (91 mg, 0.6992), the final compounds described under this example were prepared following a procedure analogous to that described for the preparation of Example 2. LC-MS for C₂₃H₂₉F₆N₃O₄ [M+H]⁺ calculated 526.21, found 526.30. The two respective cis-THP diastereoisomers were separated using a Chiralcel OD semipreparative chiral column, hexane/ethanol (85:15) mixture at a flow rate of 9 mL/min.

### EXAMPLE 4

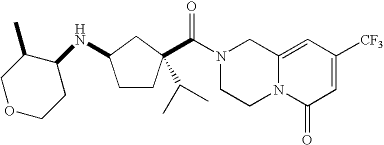 Procedure A
Step A

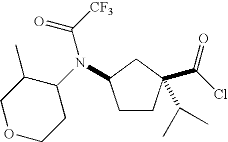

A solution of the acid Intermediate 12 (757 mg, 2.17 mmol) in dichloromethane (20 mL) was cooled to 0° C. and treated with oxalyl chloride (568 μL, 6.52), followed by three drops of DMF. The cooling bath was removed, and the reaction mixture was stirred at ambient temperature for 2 hrs, after which times a min-quench with methanol followed by HPLC analysis confirmed full conversion of the acid to the respective chloride. The reaction solvent was removed in vacuo, and the residue was Kugelrohr distilled (250° C., 0.1 mmHg) to afford the pure acid chloride (730 mg, 87%). LC-MS for C₁₈H₂₈F₃NO₄ [M+H]⁺ (methyl ester) calculated 380.20, found 380.15.
Step B

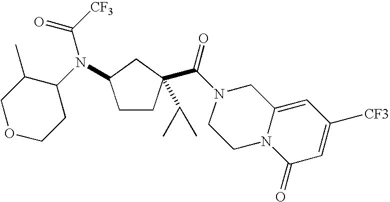

A solution of the amine Intermediate 3 (248 mg, 0.974 mmol as a hydrochloride) and diisopropylethylamine (678 μL, 3.896 mmol) in dichloromethane (10 mL) was treated at 0° C. with a solution of the acid chloride from the previous step (448 mg, 1.169 mmol) in dichloromethane (20 mL), the cooling bath was removed, and the reaction mixture was stirred at room temperature for 2 hrs. The reaction was quenched with sodium bicarbonate (saturated, aqueous, 40 mL) and the crude product was extracted with dichloromethane (654 mg). It was purified by preparative TLC using a mixture of ethyl acetate and hexanes (1:1) as an eluent to yield 309 mg (47%) of the desired product as a mixture of tetrahydropyrane-ring-derived isomers. LC-MS for C₂₆H₃₃F₆N₃O₄ [M+H]⁺ calculated 566.24, found 566.20.
Step C

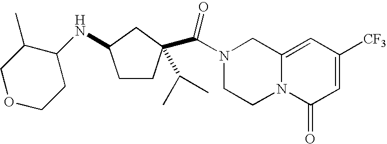

A solution of the trifluoroacetamide intermediate from the previous step (310 mg, 0.5484 mmol) in ethanol (15 mL) was treated in several portions with sodium borohydride (207 mg, 5.48 mmol). The reaction was quenched with aqueous saturated sodium bicarbonate and the product was extracted with chloroform. The combined organic extracts were dried (anhydrous sodium sulfate), and the solvent was removed in vacuo to afford the crude product (246 mg) containing a mixture of the two main tetrahydropyrane-cis-isomers as well as various partially saturated hexahydroisoquinolones.
Step D

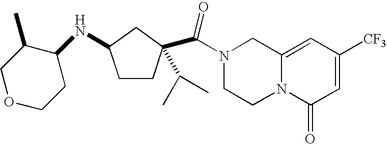

The respective isomers were separated using a Chiralpak AD semi-preparative chiral column, using a mixture of hexanes and ethanol (85:15) as an eluent, at a flow rate of 9.0 mL/min. The title compound was obtained as the faster eluting isomer (Tr=10.70 min, m=24.4 mg). LC-MS for C₂₄H₃₄F₃N₃O₃ [M+H]⁺ calculated 470.26, found 470.15.

Procedure B

Step A

Step B

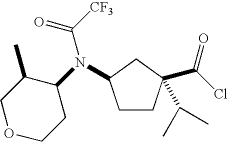

A solution of the acid Intermediate 13 (120 mg, 0.328 mmol) in dichloromethane (6 mL) was cooled to 0° C. in an inert atmosphere of nitrogen, and oxalyl chloride (85 μL, 0.9852 mmol) was added via syringe, followed by three drops of DMF. The cooling bath was removed, and the reaction mixture was stirred for 3 hrs. The solvent was removed in vacuo, and the crude acyl chloride was further purified by Kugelrohr distillation as described in this example, Procedure A, Step A to yield 170 mg of the desired chloride. The chloride was used immediately.
Step C

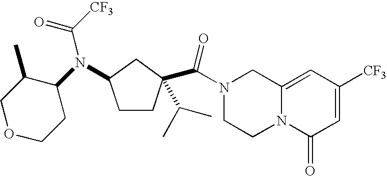

A solution of the Intermediate 3 (in a form of a hydrochloride, 84 mg, 0.328 mmol) in dichloromethane (4 mL) was treated with diisopropylethylamine (285 μL, 1.64 mmol) and to this solution was added the solution of the acyl chloride, preparation of which was described in the previous step. The reaction mixture was stirred at room temperature overnight, after which it was quenched by addition of saturated aqueous sodium bicarbonate (20 mL). The crude product was extracted with dichloromethane (3×30 mL), the combined organic extracts were concentrated (212 mg) and purified by preparative TLC (100% ethyl acetate as an eluent) to yield 70 mg (37%) of the pure product. LC-MS for C₂₆H₃₃F₆N₃O₄ [M+H]⁺ calculated 566.24, found 566.22. ¹H NMR (500 MHz, CDCl₃): 6.79 (s, 1H), 6.25 (s, 1H), 4.73 (m, 2H), 4.20, (m, 2H), 3.91 (m, 3H), 3.71 (dd, (J=11.4, 3.0 Hz, 1H), 3.50 (dd, J=11.4, 2.5 Hz, 1), 3.42 (dt, J=11.0, 3.0 Hz, 1H), 3.09 (bs, 1H), 2.90 (bs, 1H), 2.54 (bs, 1H), 2.1 (m, 1H), 1.92 (bm, 4H), 1.60 (m, 3H), 1.0 (d, J=6.9H, 3H), 0.93 (d, J=6.6 Hz, 3H), 0.86 (d, J=6.6 Hz, 3H).
Step D

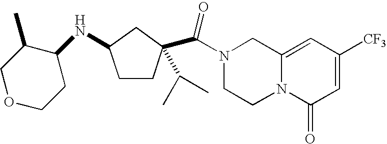

A solution of the trifluoroacetamide from the previous step (70 mg, 0.1238 mmol) in ethyl alcohol (6 mL) was treated with sodium borohydride (46 mg, 1.24 mmol) in several portions, during 3 hrs. The solvent was removed in vacuo, the residue was picked up into saturated aqueous sodium bicarbonate, and extracted several times with dichloromethane. The combined organic extracts were dried (anhydrous sodium sulfate), filtered, and concentrated in vacuo. The residue (47 mg) was purified by preparative TLC (DCM+(MeOH+NH4OH/9:1)/9:1) to afford 31.3 mg (54%) of the pure product. Its spectral and chromatographic behavior was identical to that described as faster eluting isomer, Step C, Procedure A of this example.

Procedure C

Step A

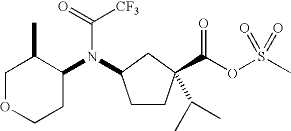

A solution of Intermediate 13 (487 mg, 1.33 mmol) and diisopropylethylamine (923 μL, 2.66 mmol) in dry THF was cooled to 0° C. and neat methanesulfonyl chloride was added via syringe. The reaction progress was monitored by periodical sampling of the reaction mixture, quenching the samples with methyl alcohol, and comparing the peak intensities of the starting acid vs. the methyl ester, produced by the mini-quench. The formation of the anhydride was complete after 1 hr at room temperature. This intermediate was used in the following step without any further workup or purification, without any unnecessary delay.
Step B

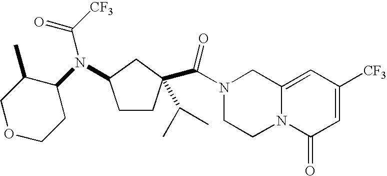

To the solution of the intermediate anhydride preparation of which was described in the previous step was added a dichloromethane solution of Intermediate 3 (380 mg, 1.59 mmol) containing diisopropylethylamine (923 μL, 2.66 mmol). The reaction mixture was stirred at room temperature another 1 hr after which it was poured onto sat. solution of NaHCO₃. The crude product was extracted into dichloromethane, the combined organic extracts were combined, dried and the solvent was removed in vacuo. The crude product was further purified as described in Procedure B, Step C of this example to yield 793 mg of pure product.
Step C

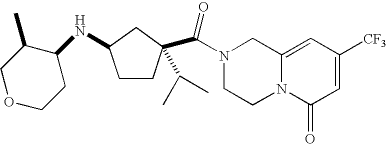

A solution of the trifluoroacetamide from the previous step (790 mg, 1.44 mmol) in ethyl alcohol (10 mL) was treated with sodium borohydride (550 mg, 14.4 mmol) in several portions. After two hrs the solvent was removed in vacuo and aqueous sodium bicarbonate (10 mL) was added and the crude product was extracted with a mixture of chloroform and isopropyl alcohol (4:1, 3×30 mL). The combined organic extracts were dried and the solvent was removed in vacuo. Further purification as achieved as described in Procedure B, Step D of this example, or by preparative chromatography using a Chiralcel OD column and a mixture of hexanes and ethyl alcohol (95:5) as an eluent. The spectral and chromatographic behavior was identical to that of a standard sample.

Procedure D

Step A

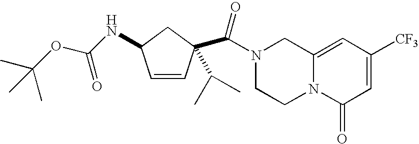

A solution of Intermediate 15 (684 mg, 1.156 mmol), BOC₂O (510 mg, 2.31 mmol) in dichloromethane (10 mL) was treated with saturated aqueous solution of sodium bicarbonate (10 mL) and vigorously stirred at room temperature for 2 hrs. The organic layer was separated, the aqueous was washed with dichloromethane (3×20 mL). The combined organic layers were back-washed with brine, dried with anhydrous sodium sulfate, filtered, and the solvent was removed in vacuo. The residue was purified by gradient column chromatography on silicagel, using a ethyl acetate-hexane mixture as an eluent. The concentration of ethyl acetate was gradually increased from 0 to 100%. In this fashion, 304 mg (56%) of desired product was obtained. ¹H NMR (500 MHz, CDCl₃): 6.8 (s, 1H), 6.3 (s, 1H), 6.07 (dd, J=5.7, 1.8 Hz, 1H), 5.80 (dd, 5.7, 1.8 Hz, 1H), 4.75 (m, 4H), 4.2 (bt, J=5.3 Hz, 2H), 3.91 (bt, 5.7 Hz, 2H), 2.68 (dd, J=13.0, 7.8 Hz, 1H), 2.1 (m, 1H), 1.86 (dd, J=13.0, 4.8 Hz, 1H), 1.43 (s, 9H), 0.88 (d, J=6.9 Hz, 3G), 0.82 (d, J=6.9 Hz, 3H).
Step B

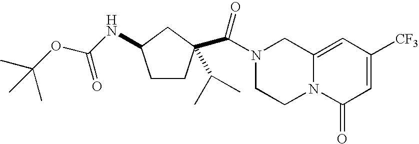

A solution of the olefin from previous step (84 mg, 0.1789 mmol) and Pd/C (83 mg, 10%) in ethyl alcohol (10 mL) was hydrogenated at ambient pressure for 2 hrs. The catalyst was filtered off and the solvent was removed under reduced pressure to leave 84 mg of the crude product. This was further purified using preparative TLC (ethyl acetate+hexanes/4:1) to afford 10.4 mg of the clean product. ¹H NMR (500 MHz, CDCl₃): 6.8 (s, 1H), 6.2 (s, 1H), 4.84 (m, 2H), 4.65 (m, 2H), 4.2 (m, 2H), 3.91 (m, 2H), 2.68 (m, 1H), 2.24 (m, 2H), 2.1 (m, 4H), 1.86 (m, 2H), 1.43 (s, 9H), 0.82 (m 7H).
Step C

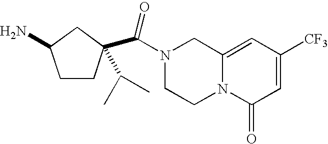

A solution of the BOC-protected amine from the previous step (275 mg, 0.584 mmol) was dissolved in 4N solution of HCl in dioxane and stirred at room temperature for 2 hrs. The solvent was removed in vacuo to yield 240.6 mg of the pure product in a form of a hydrochloride salt. LC-MS for C₁₈H₂₄F₃N₃O₂ [M+H]⁺ calculated 382.18, found 382.4.
Step D

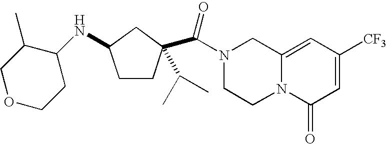

A solution of the amine preparation of which was described in the previous step (240.6 mg, 0.584 mmol), ketone Intermediate 7 (200 mg, 1.752 mmol) 4A molecular sieves (1.7 g), diisopropylethylamine (100 μL, 0.584 mmol) in dichloromethane (10 mL) was treated with sodium triacetoxyborohydride (618 mg, 2.92 mmol) and stirred at ambient temperature for 24 hrs. The reaction mixture was poured onto a saturated solution of sodium bicarbonate and the product was extracted with dichloromethane (4×50 mL). The combined extracts were dried (anhydrous sodium sulfate) and the solvent was removed in vacuo. The residue (270 mg) was purified by preparative TLC, using a dichloromethane+methanol+ammonium hydroxide/90:9:1 mixture as an eluent. This diastereoisomeric mixture appeared as a single peak on a reverse phase chromatographic analysis, and a mass spectrometric analysis of this peak confirmed the expected molecular weight: LC-MS for C₂₄H₃₄F₃N₃O₃ [M+U]⁺ calculated 470.26, found 470.50.
Step E

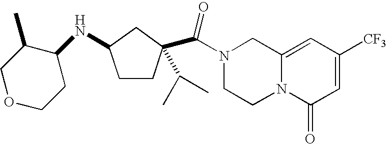

This single isomer was obtained by a semipreparative chiral chromatography using a Chiralcel OD column, eluted with a 87:13 mixture of hexanes and ethyl alcohol, and a flowrate of 9.0 mL/min. Under these conditions the title compound eluted as the third chromatographic peak with an analytical (analytical Chiralcel column, identical eluent, flow rate of 1.0 mL/min) retention time of 47.4 minutes. All spectral as well as chromatographic parameters recorded for this sample matched those obtained for the independently synthesized standard.

### EXAMPLE 5

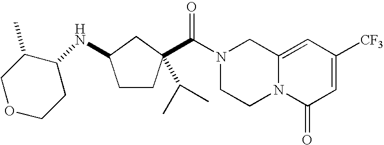 Procedure A

The title compound was obtained by a chromatographic separation from a mixture of isomers, preparation of which was described under Example 4, steps A-C, using a semi-preparative Chiralpak AD column. The employed conditions described under Example 4, Step C and the title compound was obtained as the slower eluting isomer (Tr=12.01 min, m=25.4 mg).

Procedure B

Step A

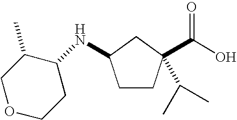

This acid was prepared starting from ester Intermediate 11 in a procedure analogous to that described for Intermediate 13, Procedure A.
Step B

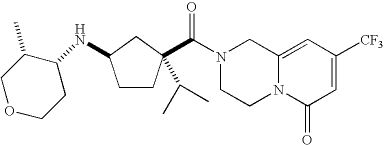

The title compound was synthesized in a way identical to that described under Example 4, Procedure B, Steps A through C, except that the Intermediate 11 was used as starting material. Its spectral and chromatographic behavior was identical to that of the slower eluting isomer, preparation of which was described in Example 4, Step C.

Procedure C

This single isomer was obtained by a semipreparative chiral chromatography using a Chiralcel OD column, eluted with a 87:13 mixture of hexanes and ethyl alcohol, and a flowrate of 9.0 mL/min. Under these conditions the title compound eluted as the fourth chromatographic peak with an analytical (analytical Chiralcel column, identical eluent, flow rate of 1.0 mL/min) retention time of 51.4 minutes. All spectral as well as chromatographic parameters recorded for this sample matched those obtained for the independently synthesized standard.

### EXAMPLE 6

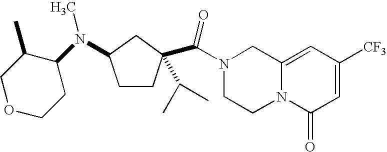

A solution of the amine from Example 4 (22.7 mg, 0.0483 mmol), formaldehyde (100 μL, 0.96 mmol), diisopropylethylamine (9 mL, 0.0483 mmol) and crushed, 4A molecular sieves (400 mg) in dichloromethane (6 mL) was treated with sodium triacetoxyborohydride (53 mg, 0.24 mmol) and stirred at room temperature overnight. The reaction was quenched by pouring onto sat. aqueous solution of sodium bicarbonate (10 mL), and the crude product was extracted with dichloromethane. (4×10 mL). The combined organic extracts were dried, and the solvent was removed in vacuo. Further purification by preparative TLC (dichloromethane:methanol:ammonium hydroxide (90:9:1) gave 7.2 mg of the desired product. LC-MS for C₂₅H₃₆F₃N₃O₃ [M+H]⁺ calculated 484.27, found 484.60.

### EXAMPLE 7

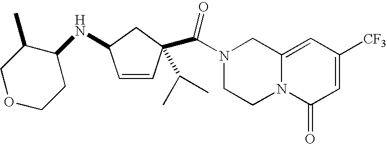 Step A

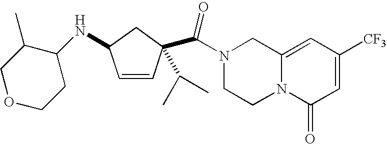

A solution of the amine Intermediate 15 (2.00 g, 5.41 mmol), ketone Intermediate 7 (1.00 g, 8.13 mmol), 4 A crushed molecular sieves (4 g) in dichloromethane (20 mL) was treated with sodium triacetoxyborohydride (3.44 g, 16.22 mmol) and stirred at room temperature for 24 hrs. The reaction mixture was poured onto a saturated solution of sodium bicarbonate (80 mL) and the product was extracted with chloroform (5×80 mL). The combined organic extracts were dried and the solvent was removed under reduced pressure. The residue (2.72 g, heavy oil) was further purified by column chromatography (silicagel, dichloromethane+methanol+ammonium hydroxide (90:9:1) to afford 1.2979 g of a diastereoisomeric mixture of the two respective cis-diastereoisomers.
Step B

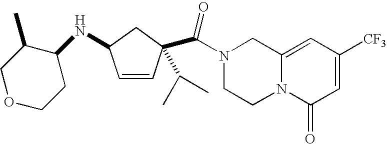

The mixture of the two respective cis-diastereoisomers was separated into the title (THP-3S,4S) isomer by a chiral semipreparative Chiralcel OD column, using a mixture (9:1) of hexanes and ethyl alcohol as an eluent and a flow rate of 9.0 mL/min. Under analogous analytical conditions (1.0 mL flow rate, identical column) the title isomer eluted first, Tr=23.30 mins, while the respective THP-3R,4R isomer (Example 8) eluted second, Tr=25.65 min.

### EXAMPLE 8

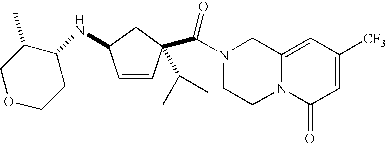

The title compound was obtained as the second eluting isomer, in a separation described under Example 7.

### EXAMPLE 9

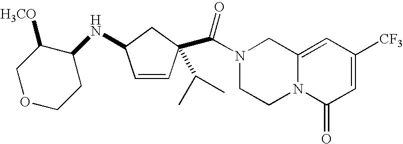

The title compound was synthesized starting from Intermediate 15 and Intermediate 5 according to a procedure analogous to that described for preparation of Example 7. Small amounts of the other diastereomeric THP-cis-isomers (for structure see Example 10) could be separated using a chiral semipreparative HPLC separation analogous to that described in Example 8. LC-MS for C₂₄H₃₂F₃N₃O₄ [M+H]⁺ calculated 484.23, found 484.50. ¹H NMR (500 MHz, CDCl₃): 6.80 (s, 1H), 6.28 (s, 1H), 6.03 (dd, J=6.0, 1.8 Hz, 1H), 5.91 (dd, J 6.0, 1.8 Hz, 1H), 4.75 (s, 2H), 4.26 (m, 1H), 4.13 (m, 1H), 4.03 (dd, J=12.1, 3.4 Hz, 1H), 3.95 (m, 4H), 3.34 (m, 6H), 2.80 (m, 1H), 2.44 (dd, J=13.3, 7.8 Hz, 1H), 2.14 (m, 1H), 1.8 (bm, 7H), 1.3 (m, 1H), 0.87 (m, 7H).

### EXAMPLE 10

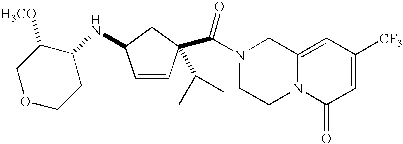

Small quantities of this isomer could be obtained via a semipreparative chiral chromatographic separation as described in Example 9. LC-MS for C₂₄H₃₂F₃N₃O₄ [M+H]⁺ calculated 484.23, found 484.50.

### EXAMPLE 11

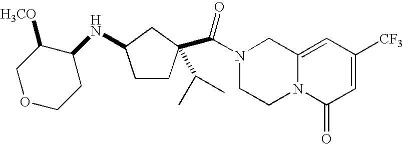

A solution of the olefin from Example 9 (69 mg, 0.144 mmol) and Pd/C (51 mg, 10%) in ethyl alcohol was hydrogenated at ambient pressure and temperature. The catalyst was filtered off, and the filtrate was evaporated to dryness. If necessary, passage through a semipreparative chiral HPLC column (Chiralcel OD, 80% hexanes, 20% ethanol) can be used to remove isomeric contaminants. LC-MS for C₂₄H₃₄F₃N₃O₄ [M+H]⁺ calculated 486.25, found 486.55.

### EXAMPLE 12

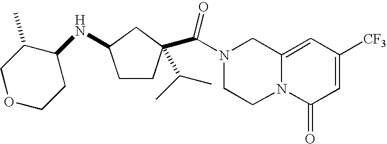

This single isomer was obtained from a isomeric mixture, preparation of which was described under Example 4 Procedure D by a semipreparative chiral chromatography using a Chiralcel OD column, eluted with a 87:13 mixture of hexanes and ethyl alcohol, and a flowrate of 9.0 mL/min. Under these conditions the title compound eluted as the first chromatographic peak with an analytical (analytical Chiralcel column, identical eluent, flow rate of 1.0 mL/min) retention time of 29.4 minutes.

### EXAMPLE 13

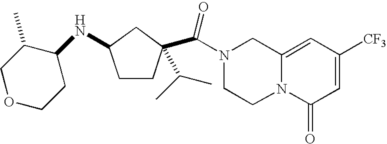

This single isomer was obtained from a isomeric mixture, preparation of which was described under Example 4 Procedure D by a semipreparative chiral chromatography using a Chiralcel OD column, eluted with a 87:13 mixture of hexanes and ethyl alcohol, and a flowrate of 9.0 mL/min. Under these conditions the title compound eluted as the second chromatographic peak with an analytical (analytical Chiralcel column, identical eluent, flow rate of 1.0 mL/min) retention time of 34.2 minutes.

### EXAMPLE 14

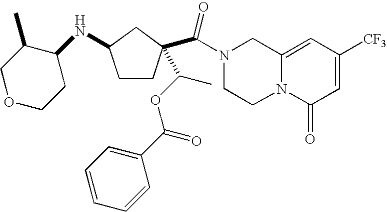 Step A

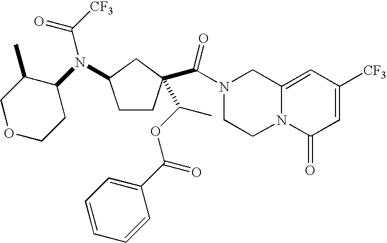

A solution of the acid Intermediate 14 (171 mg, 0.3016 mmol), diisopropylethylamine (105 μL, 0.6032 mmol) in THF was cooled to 0° C. and neat methanesulfonyl chloride (302 μL, 0.3016 mmol) was added. The stirring at 0° C. was continued, until a mini-quench with methanol, analyzed by HPLC indicated a full formation of the methylester, a sign, that the mixed anhydride was fully formed. To this solution, a mixture of Intermediate 3 (77 mg, 0.3016 mmol, as a hydrochloride salt), triethylamine (105 μL, 0.6032 mmol) in THF (2 mL) was added, and the cooling bath was removed. Stirring at room temperature was continued for an additional 1 hour. The solvent was removed in vacuo, water (20 mL) was added, and the product was extracted into dichloromethane (4×20 mL). The combined organic extracts were dried (anhydrous sodium sulfate) filtered, and the solvent was removed under reduced pressure. The residue was purified by preparative TLC to afford 24 mg of the pure desired product. LC-MS for C₃₂H₃₅F₆N₃O₆ [M+H]⁺ calculated 672.24, found 672.25.
Step B

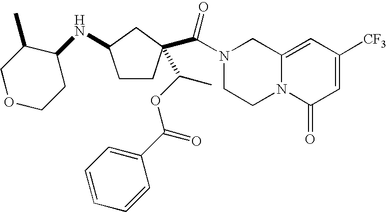

A solution of the trifluoroacetamide from the previous step (25 mg, 0.0372 mmol) in ethanol (6 mL) was treated with sodium borohydride (38 mg, 1 mmol) in small portions, and stirred at room temperature for 2 hrs. The solvent was removed in vacuo, the residue was purified by preparative TLC, using dichloromethane:methanol:ammonium hydroxide (90:9:1) as an eluent. In this fashion 15.4 mg of the pure product was obtained. LC-MS for C₃₀H₃₆F₃N₃O₅ [M+H]⁺ calculated 576.26, found 576.30.

### EXAMPLE 15

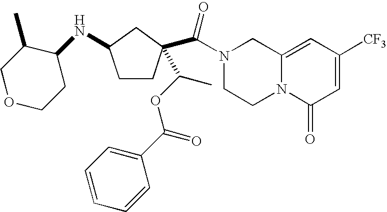

A solution of the ester from Example 14 (15 mg, 0.0261 mmol) in methanol (3.0 mL) was treated with aqueous solution of lithium hydroxide (600 μL, 1N) and stirred at ambient temperature for 3 hrs. The volatiles were removed under reduced pressure, and the product was extracted with dichloromethane. The combined organic phases were dried with anhydrous sodium sulfate, filtered, and the solvent was evaporated in vacuo. The residue was purified by preparative TLC, using dichloromethane:methanol:ammonium hydroxide (90:9:1) as an eluent. In this fashion, 5.4 mg of the desired product was obtained. LC-MS for C23H32F3N3O4 [M+H]+calculated 472.23, found 472.25.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of Formula I or Formula II: 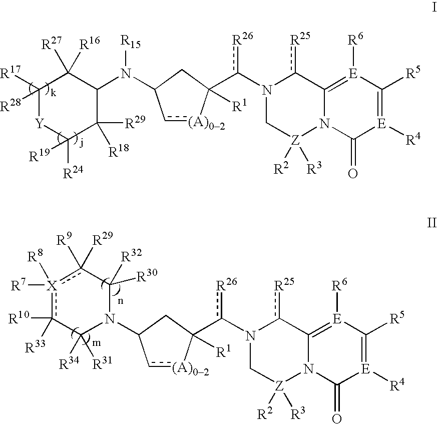
wherein:
A is selected from: —CH₂—, —O—, —N(R²⁰)—, —S—, —SO—, —SO₂—, —N(SO₂R¹⁴)—, and —N(COR¹³)—;
E is independently selected from N and C;
X is O, N, S, SO₂ or C;
Y is selected from: —O—, —N(R²⁰)—, —S—, —SO—, —SO₂—, and —C(R²¹)(R²²)—, —N(SO₂R¹⁴)—, —N(COR¹³)—, —C(R²¹)(COR¹¹)—, —C(R²¹)(OCOR¹⁴)— and —CO—;
Z is selected from C, N or O;
R¹ is selected from: hydrogen, —C₁₋₆alkyl, —O—C₁₋₆alkyl, —S—C₁₋₆alkyl, —SO—C₁₋₆alkyl, —SO₂—C₁₋₆alkyl, —SO₂NR¹²R¹², —NR¹²—SO₂—NR¹²R¹², —(C₀₋₆alkyl)-(C₃₋₇cycloalkyl)-(C₀₋₆alkyl), —CN, —NR¹²R¹², —NR¹²COR¹³, —NR¹²SO₂R¹⁴, —COR¹¹, —CONR¹²R¹², —NR¹²CONR¹²R¹², —O—CO—C₁₋₆alkyl, —O—CO₂—C₁₋₆alkyl, hydroxy, heterocycle and phenyl,
where said alkyl and cycloalkyl are unsubstituted or substituted with 1-7 substituents independently selected from: halo, hydroxy, —O—C₁₋₆alkyl unsubstituted or substituted with 1-6 fluoro, C₁₋₆alkyl unsubstituted or substituted with 1-6 fluoro, —CONR¹²R¹², —NR¹²CONR¹²R¹², —COR¹¹, —SO₂R¹⁴, —NR¹²COR¹³, —NR¹²SO₂R¹⁴, -heterocycle, ═O, —CN, phenyl, —SO₂NR¹²R¹², —NR¹²—SO₂—NR¹²R¹², —S—C₁₋₆alkyl unsubstituted or substituted with 1-6 fluoro, —SO—C₁₋₆alkyl unsubstituted or substituted with 1-6 fluoro, —SO₂—C₁₋₆alkyl, unsubstituted or substituted with 1-6 fluoro, and —O—COR¹³,
where said phenyl and heterocycle are unsubstituted or substituted with 1-3 substituents independently selected from: halo, hydroxy, —COR¹¹, C₁₋₃alkyl, and C₁₋₃alkoxy, said C₁₋₃alkyl and C₁₋₃alkoxy being unsubstituted or substituted with 1-6 fluoro;
R² and R³ are nothing when Z is O;
R² is nothing and R³ is hydrogen or C₁₋₃alkyl when Z is N;
R² and R³ are independently hydrogen or C₁₋₃alkyl unsubstituted or substituted with 1-3 fluoro, when Z is C;
R⁴ is selected from: hydrogen, C₁₋₃alkyl unsubstituted or substituted with 1-3 fluoro, —O—C₁₋₃alkyl unsubstituted or substituted with 1-3 fluoro, hydroxy, chloro, fluoro, bromo, phenyl and heterocycle, when E is C;
R⁵ is selected from: fluoro, chloro, bromo, -heterocycle, —CN, —COR¹¹, C₄₋₆cycloalkyl, —O—C₄₋₆cycloalkyl, C₁₋₆alkyl unsubstituted or substituted with 1-6 fluoro or hydroxyl or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from halo, trifluoromethyl, C₁₋₄alkyl and COR¹¹, -phenyl unsubstituted or substituted with one or more substituents selected from halo, trifluoromethyl, C₁₋₄alkyl and COR¹¹, —O—phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, and —O—C₃₋₆cycloalkyl unsubstituted or substituted with 1-6 fluoro, when E is C;
R⁶ is selected from: hydrogen, hydroxy, chloro, fluoro, bromo, phenyl, heterocycle, C₁₋₃alkyl unsubstituted or substituted with 1-3 fluoro and —O—C₁₋₃alkyl unsubstituted or substituted with 1-3 fluoro, when E is C;
R⁴ and R⁶ are independently selected from nothing or O (to make an N-oxide) when E is N;
R⁷ is selected from: hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W—C₀₋₄alkyl, (C₀₋₆alkyl)-CONR¹²-phenyl and (C₀₋₆alkyl)-CONR²³—V—COR¹¹, when X is N or C,
where W is selected from: a single bond, —O—, —S—, —SO—, —SO₂—, —CO—, —CO₂—, —CONR¹²— and —NR¹²—,
where V is selected from C₁₋₆alkyl or phenyl,
where R²³ is hydrogen or C₁₋₄alkyl, or R²³ is a 1-5 carbon linker to one of the carbons of V to form a ring,
where said C₀₋₆alkyl is unsubstituted or substituted with 1-5 substituents independently selected from: halo, hydroxy, —C₀₋₆alkyl, —O—C₁₋₃alkyl, trifluoromethyl and —C₀₋₂alkyl-phenyl,
where said phenyl, heterocycle, cycloalkyl and C₀₋₄alkyl, if present, are unsubstituted or substituted with 1-5 substituents independently selected from: halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃-heterocycle,
or where said phenyl or heterocycle is fused to another heterocycle, said other heterocycle being unsubstituted or substituted with 1-2 substituents independently selected from hydroxy, halo, —COR¹¹, and —C₁₋₃alkyl,
and where alkene is unsubstituted or substituted with 1-3 substituents which are independently selected from: halo, trifluoromethyl, C₁₋₃alkyl, phenyl and heterocycle;
R⁷ is absent when X is O, S, or SO₂;
R⁸ is selected from: hydrogen, hydroxy, C₁₋₆alkyl, C₁₋₆alkyl-hydroxy, —O—C₁₋₃alkyl, —COR¹¹, —CONR¹²R¹² and —CN, when X is C;
R⁸ is nothing, when X is O, S, SO₂ or N, or when a double bond joins the carbons to which R⁷ and R¹⁰ are attached;
or, R⁷ and R⁸ are joined to form a ring selected from: 1H-indene, 2,3-dihydro-1H-indene, 2,3-dihydro-benzofuran, 1,3-dihydro-isobenzofuran, 2,3-dihydro-benzothiofuran, 1,3-dihydro-isobenzothiofuran, 6H-cyclopenta[d]isoxazol-3-ol, cyclopentane and cyclohexane,
where said ring is unsubstituted or substituted with 1-5 substituents independently selected from:
halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃alkyl-heterocycle;
R⁹ and R¹⁰ are independently selected from: hydrogen, hydroxy, C₁₋₆alkyl, C₁₋₆alkyl-COR¹¹, C₁₋₆alkyl-hydroxy, —O—C₁₋₃alkyl, halo;
or R⁹ and R¹⁰ together are O, where O is connected to the ring via a double bond;
or, R⁷ and R⁹, or R⁸ and R¹⁰, are joined to form a fused ring which is phenyl or heterocycle, wherein said fused ring is unsubstituted or substituted with 1-7 substituents independently selected from: halo, trifluoromethyl, hydroxy, C₁₋₃alkyl, —O—C₁₋₃alkyl, —COR¹¹, —CN, —NR¹²R¹² and —CONR¹²R¹²;
R¹¹ is independently selected from: hydroxy, hydrogen, C₁₋₆ alkyl, —O—C₁₋₆alkyl, benzyl, phenyl, C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl, and trifluoromethyl;
R¹² is selected from: hydrogen, C₁₋₆ alkyl, benzyl, phenyl and C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl, and trifluoromethyl;
or, when two separate R¹² groups reside on the same atom or adjacent atoms, said two R¹² groups are optionally connected via a C₁₋₇alkyl linker to form a 3 to 9 membered ring, said linker being unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
R¹³ is selected from: hydrogen, C₁₋₆ alkyl, —O—C₁₋₆alkyl, benzyl, phenyl and C₃₋₆ cycloalkyl, where said alkyl, phenyl, benzyl, and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
R¹⁴ is selected from: hydroxy, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
R¹⁵ is hydrogen or C₁₋₆alkyl, where said alkyl is unsubstituted or substituted with 1-3 substituents independently selected from: halo, hydroxy, —CO₂H, —CO₂C₁₋₆alkyl, and —O—C₁₋₃alkyl;
R¹⁶ is selected from: hydrogen, fluoro, C₃₋₆ cycloalkyl, —O—C₃₋₆cycloalkyl, hydroxy, —COR¹¹, —OCOR¹⁴, C₁₋₆alkyl unsubstituted or substituted with 1-6 substituents selected from fluoro, C₁₋₃alkoxy, hydroxyl and —COR¹¹, and —O—C₁₋₃alkyl unsubstituted or substituted with 1-3 fluoro;
or, R¹⁵ and R¹⁶ together are a C₂₋₄alkyl or a C₀₋₂alkyl-O—C₁₋₃alkyl, forming a ring where said ring has 5-7 members;
R¹⁷ is selected from: hydrogen, COR¹¹, hydroxy, —O—C₁₋₆alkyl unsubstituted or substituted with 1-6 substituents selected from fluoro, C₁₋₃alkoxy, hydroxy, and —COR¹¹ and C₁₋₆alkyl unsubstituted or substituted with 1-6 substituents selected from fluoro, C₁₋₃alkoxy, hydroxy, and —COR¹¹, or R¹⁷ is nothing if R²⁸ is connected to a ring carbon via a double bond;
or, R¹⁶ and R¹⁷ together are C₁₋₄alkyl or C₀₋₃alkyl-O—C₀₋₃alkyl, forming ring where said ring has 3-7 members;
R¹⁸ is selected from: hydrogen, fluoro, —O—C₃₋₆cycloalkyl, —O—C₁₋₃alkyl unsubstituted or substituted with 1-6 fluoro and C₁₋₆alkyl unsubstituted or substituted with 1-6 fluoro;
or, R¹⁶ and R¹⁸ together are C₂₋₃alkyl, where said alkyl is unsubstituted or substituted with 1-3 substituents independently selected from: halo, hydroxy, —COR¹¹, C₁₋₃alkyl, and C₁₋₃alkoxy;
or, R¹⁶ and R¹⁸ together are C₁₋₂alkyl-O—C₁₋₂alkyl, where said alkyl is unsubstituted or substituted with 1-3 substituents independently selected from: halo, hydroxy, —COR¹¹, C₁₋₃alkyl, and C₁₋₃alkoxy;
or, R¹⁶ and R¹⁸ together are —O—C₁₋₂alkyl-O—, where said alkyl is unsubstituted or substituted with 1-3 substituents independently selected from halo, hydroxy, —COR¹¹, C₁₋₃alkyl, and C₁₋₃alkoxy;
R¹⁹ is selected from: hydrogen, COR¹¹, SO₂R¹⁴, SO₂NR¹²R¹² and C₁₋₃alkyl unsubstituted or substituted with 1-6 substituents independently selected from fluoro and hydroxyl;
R²⁰ is selected from: hydrogen, C₁₋₆ alkyl, benzyl, phenyl and C₃₋₆ cycloalkyl, where said alkyl, phenyl, benzyl and cycloalkyl groups are unsubstituted or substituted with 1-6 substituents independently selected from halo, hydroxy, C₁₋₃alkyl, C₁₋₃alkoxy, —CO₂H, —CO₂—C₁₋₆ alkyl, and trifluoromethyl;
R²¹ and R²² are independently selected from: hydrogen, hydroxy, C₁₋₆ alkyl, —O—C₁₋₆alkyl, benzyl, phenyl and C₃₋₆ cycloalkyl where said alkyl, phenyl, benzyl, and cycloalkyl groups can be unsubstituted or substituted with 1-6 substituents independently selected from: halo, hydroxy, C₁₋₃alkyl, C₁₋₃alkoxy, —CO₂H, —CO₂—C₁₋₆ alkyl and trifluoromethyl;
R²⁴ is selected from: hydrogen, COR¹¹, SO₂R¹⁴, SO₂NR¹²R¹² and C₁₋₃alkyl, where said alkyl is unsubstituted or substituted with 1-6 substituents independently selected from: fluoro and hydroxyl;
or, R²⁴ and R¹⁷ together are a C₁₋₃alkyl bridge;
R²⁵ and R²⁶ are independently selected from: ═O where R²⁵ and/or R²⁶ is oxygen and is connected via a double bond, hydrogen, phenyl, and C₁₋₆alkyl substituted or unsubstituted with 1-6 substituents selected from —COR¹¹, hydroxy, fluoro, chloro and C₁₋₃alkyl;
R²⁷ is selected from: hydrogen, COR¹¹, SO₂R¹⁴, SO₂NR¹²R¹² and C₁₋₃alkyl, where said alkyl is unsubstituted or substituted with 1-6 substituents independently selected from fluoro and hydroxyl;
R²⁸ is selected from selected from: hydrogen, hydroxy, halo, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹² and ═O where R²⁸ is connected to the ring via a double bond and where R¹⁷ at the same position is absent;
R²⁹ and R³³ are selected from: hydrogen, hydroxy, C₁₋₆alkyl, C₁₋₆alkyl-COR¹¹, C₁₋₆alkyl-hydroxy, —O—C₁₋₃alkyl, trifluoromethyl and halo, or R²⁹ or R³³ are independently absent if the site of substitution is unsaturated;
or, R²⁹ and R¹⁶ together are a C₁₋₃alkyl bridge;
R³⁰ and R³¹ are independently selected from: hydroxy, C₁₋₆alkyl, C₁₋₆alkyl-COR¹¹, C₁₋₆alkyl-hydroxy, —O—C₁₋₃alkyl, halo and hydrogen, where said alkyl are unsubstituted or substituted with 1-6 substituents independently selected from fluoro and hydroxyl;
or, R³⁰ and R³¹ together are a —C₁₋₄alkyl-, —C₀₋₂alkyl-O—C₁₋₃alkyl- or —C₁₋₃alkyl-O—C₀₋₂alkyl-, where said alkyl are unsubstituted or substituted with 1-2 substituents consisting of oxy where the oxygen is joined to the bridge via a double bond, fluoro, hydroxy, methoxy, methyl or trifluoromethyl;
R³² and R³⁴ are independently selected from: hydrogen, hydroxy, C₁₋₆alkyl, C₁₋₆alkyl-COR¹¹, C₁₋₆alkyl-hydroxy, —O—C₁₋₃alkyl, trifluoromethyl and halo;
j is 0, 1, or 2;
k is 0, 1, or 2;
m is 0, 1, or 2;
n is 1 or 2;
the dashed line represents an optional single bond;
and pharmaceutically acceptable salts thereof and individual diastereomers thereof.

2. The compound of claim 1 of the Formula Ia: 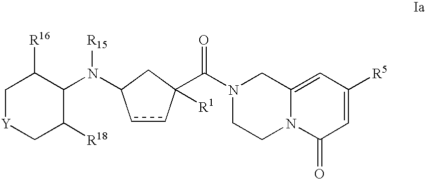
and pharmaceutically acceptable salts thereof and individual diastereomers thereof.

3. The compound of claim 1 of the Formula Ib: 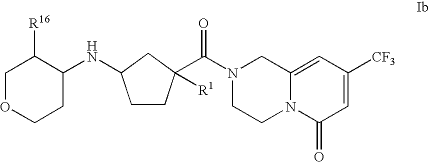
and pharmaceutically acceptable salts thereof and individual diastereomers thereof.

4. The compound of claim 1, wherein: A is CH₂, and pharmaceutically acceptable salts thereof and individual diastereomers thereof.

5. The compound of claim 1, wherein Y is O or CH₂, and pharmaceutically acceptable salts thereof and individual diastereomers thereof.

6. The compound of claim 1, wherein E is C, and pharmaceutically acceptable salts thereof and individual diastereomers thereof.

7. The compound of claim 1, wherein Z is C, and pharmaceutically acceptable salts thereof and individual diastereomers thereof.

8. The compound of claim 1, wherein R¹ is selected from: —C₁₋₆alkyl, —C₀₋₆alkyl-O—C₁₋₆alkyl, heterocycle, and -(C₀₋₆alkyl)-(C₃₋₇cycloalkyl)-(C₀₋₆alkyl), where said alkyl, heterocycle and cycloalkyl are unsubstituted or substituted with 1-7 substituents independently selected from halo, hydroxy, —O—C₁₋₃alkyl, trifluoromethyl, C₁₋₃alkyl, —O—C₁₋₁₃alkyl, —COR¹¹, —CN, —NR¹²R¹², —CONR¹²R¹² and —NCOR¹³, and pharmaceutically acceptable salts thereof and individual diastereomers thereof.

9. The compound of claim 1, wherein R¹ is selected from: C₁₋₆alkyl, C₁₋₆alkyl substituted with hydroxy, and C₁₋₆alkyl substituted with 1-6 fluoro, and pharmaceutically acceptable salts thereof and individual diastereomers thereof.

10. The compound of claim 1, wherein R¹ is selected from: —CH(CH₃)₂, —C(OH)(CH₃)₂, —CH(OH)CH₃ and —CH₂CF₃, and pharmaceutically acceptable salts thereof and individual diastereomers thereof.

11. The compound of claim 1, wherein one or more of R², R³ and R⁴ is hydrogen, and pharmaceutically acceptable salts thereof and individual diastereomers thereof.

12. The compound of claim 1, wherein R⁵ is selected from: C₁₋₆alkyl substituted with 1-6 fluoro, —O—C₁₋₆alkyl substituted with 1-6 fluoro, chloro, bromo and phenyl, and pharmaceutically acceptable salts thereof and individual diastereomers thereof.

13. The compound of claim 12, wherein R⁵ is trifluoromethyl, and pharmaceutically acceptable salts thereof and individual diastereomers thereof.

14. The compound of claim 1, wherein R¹⁵ is methyl or hydrogen, and pharmaceutically acceptable salts thereof and individual diastereomers thereof.

15. The compound of claim 1, wherein R¹⁶ is selected from: hydrogen, C₁₋₃alkyl which is unsubstituted or substituted with 1-6 fluoro, —O—C₁₋₃alkyl, fluoro and hydroxy, and pharmaceutically acceptable salts thereof and individual diastereomers thereof.

16. The compound of claim 1, wherein R¹⁶ is selected from: hydrogen, trifluoromethyl, methyl, methoxy, ethoxy, ethyl, fluoro and hydroxy, and pharmaceutically acceptable salts thereof and individual diastereomers thereof.

17. The compound of claim 1, wherein R¹⁷ is hydrogen, and pharmaceutically acceptable salts thereof and individual diastereomers thereof.

18. The compound of claim 1, wherein R¹⁸ is selected from: hydrogen, methyl, and methoxy, and pharmaceutically acceptable salts thereof and individual diastereomers thereof.

19. The compound of claim 1, R¹⁶ and R¹⁸ together are —CH₂CH₂— or —CH₂CH₂CH₂—, and pharmaceutically acceptable salts thereof and individual diastereomers thereof.

20. The compound of claim 1, wherein one or more of R¹⁹, R²⁴ and R²⁵ is hydrogen, and pharmaceutically acceptable salts thereof and individual diastereomers thereof.

21. The compound of claim 1, wherein R²⁶ is O, and pharmaceutically acceptable salts thereof and individual diastereomers thereof.

22. The compound of claim 1, wherein one or more of R²⁷, R²⁸ and R²⁹ is hydrogen, and pharmaceutically acceptable salts thereof and individual diastereomers thereof.

23. A compound selected from: 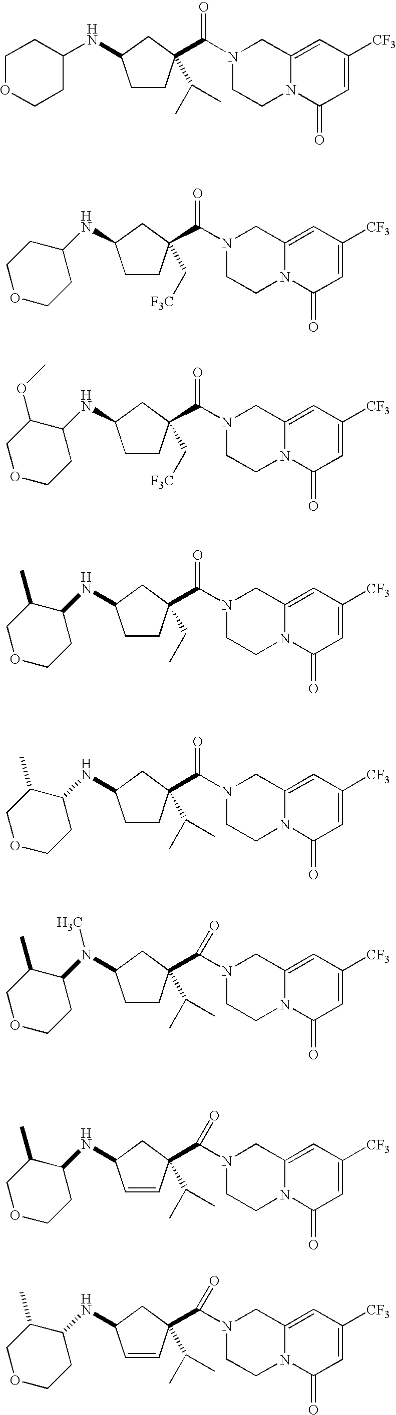 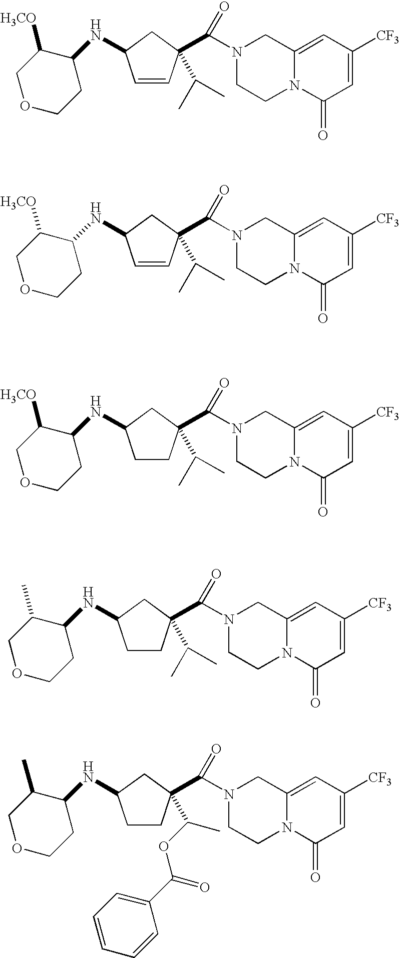
and pharmaceutically acceptable salts thereof and individual diastereomers thereof.

24. A pharmaceutical composition which comprises an inert carrier and a compound of claim 1.

25. A method for modulations of chemokine receptor activity in a mammal which comprises the administration of an effective amount of a compound of claim 1.

26. A method for treating, ameliorating, controlling or reducing the risk of an inflammatory and immunoregulatory disorder or disease which comprises the administration to a patient of an effective amount of a compound of claim 1.

27. A method for treating, ameliorating, controlling or reducing the risk of rheumatoid arthritis which comprises the administration to a patient of an effective amount of a compound of claim 1.
